SEQUENCE LISTING (45 sequences)

SEQ ID NO: 1 - protein, 15 aa, Homo sapiens
GGGGSGGGGSGGGGS

SEQ ID NO: 2 - protein, 15 aa, Homo sapiens
ESGRSGGGGSGGGGS

SEQ ID NO: 3 - protein, 14 aa, Homo sapiens
EGKSSGSGSESKST

SEQ ID NO: 4 - protein, 15 aa, Homo sapiens
EGKSSGSGSESKSTQ

SEQ ID NO: 5 - protein, 14 aa, Homo sapiens
EGKSSGSGSESKVD

SEQ ID NO: 6 - protein, 14 aa, Homo sapiens
GSTSGSGKSSEGKG

SEQ ID NO: 7 - protein, 18 aa, Homo sapiens
KESGSVSSEQLAQFRSLD

SEQ ID NO: 8 - protein, 16 aa, Homo sapiens
ESGSVSSEELAFRSLD

SEQ ID NO: 9 - protein, 4 aa, Homo sapiens
KDEL

SEQ ID NO: 10 - protein, 4 aa, Homo sapiens
DDEL

SEQ ID NO: 11 - protein, 4 aa, Homo sapiens
DEEL

SEQ ID NO: 12 - protein, 4 aa, Homo sapiens
QEDL

SEQ ID NO: 13 - protein, 4 aa, Homo sapiens
RDEL

SEQ ID NO: 14 - protein, 7 aa, Homo sapiens
PKKKRKV

SEQ ID NO: 15 - protein, 7 aa, Homo sapiens
PQKKIKS

SEQ ID NO: 16 - protein, 5 aa, Homo sapiens
QPKKP

SEQ ID NO: 17 - protein, 4 aa, Homo sapiens
RKKR

SEQ ID NO: 18 - protein, 5 aa, Homo sapiens
KKKRK

SEQ ID NO: 19 - protein, 12 aa, Homo sapiens
RKKRRQRRRAHQ

SEQ ID NO: 20 - protein, 16 aa, Homo sapiens
RQARRNRRRRWRERQR

SEQ ID NO: 21 - protein, 19 aa, Homo sapiens
MPLTRRRPAASQALAPPTP

SEQ ID NO: 22 - protein, 15 aa, Homo sapiens
MDDQRDLISNNEQLP

SEQ ID NO: 23 - protein, 32 aa, Homo sapiens
  misc_feature (7)..(8): Xaa can be any naturally occurring amino acid
MLFNLRXXLNNAAFRHGHNFMVRNFRCGQPLX

SEQ ID NO: 24 - protein, 3 aa, Homo sapiens
AKL

SEQ ID NO: 25 - protein, 6 aa, Homo sapiens
SDYQRL

SEQ ID NO: 26 - protein, 8 aa, Homo sapiens
GCVCSSNP

SEQ ID NO: 27 - protein, 8 aa, Homo sapiens
GQTVTTPL

SEQ ID NO: 28 - protein, 8 aa, Homo sapiens
GQELSQHE

SEQ ID NO: 29 - protein, 8 aa, Homo sapiens
GNSPSYNP

SEQ ID NO: 30 - protein, 8 aa, Homo sapiens
GVSGSKGQ

SEQ ID NO: 31 - protein, 8 aa, Homo sapiens
GQTITTPL

SEQ ID NO: 32 - protein, 8 aa, Homo sapiens
GQTLTTPL

SEQ ID NO: 33 - protein, 8 aa, Homo sapiens
GQIFSRSA

SEQ ID NO: 34 - protein, 8 aa, Homo sapiens
GQIHGLSP

SEQ ID NO: 35 - protein, 8 aa, Homo sapiens
GARASVLS

SEQ ID NO: 36 - protein, 8 aa, Homo sapiens
GCTLSAEE

SEQ ID NO: 37 - protein, 16 aa, Homo sapiens
AAVALLPAVLLALLAP

SEQ ID NO: 38 - protein, 12 aa, Homo sapiens
AAVLLPVLLAAP

SEQ ID NO: 39 - protein, 15 aa, Homo sapiens
VTVLALGALAGVGVG

SEQ ID NO: 40 - DNA, 21 bp, Homo sapiens
atggagctccaggcagcccgc

SEQ ID NO: 41 - DNA, 23 bp, Artificial Sequence
  Description of artificial sequence  PCR primer
gccatacgggtgtgtgagccagc

SEQ ID NO: 42 - DNA, 25 bp, Artificial Sequence
  Description of artificial sequence  PCR primer
cagtggtggacctgacctgccgtct

SEQ ID NO: 43 - DNA, 27 bp, Artificial Sequence
  Description of artificial sequence  PCR primer
ctcagtgtagcccaggatgcccttgag

SEQ ID NO: 44 - DNA, 31 bp, Artificial Sequence
  Description of artificial sequence phosphorothioate-modified antisense oligonucleotides
ccagcagtaccgcttccttgccctgcggccg

SEQ ID NO: 45 - DNA, 30 bp, Artificial Sequence
  Description of artificial sequence phosphorothioate-modified antisense oligonucleotides
gccgcgtcccgttccttcaccatgacgacc

(19) United States
(12) Patent Application Publication
(10) Pub. No.: US 2005/0059592 A1
(43) Pub. Date: Mar. 17, 2005

(54) EphA2 and hyperproliferative cell disorders

(76) Inventors: Peter A. Kiener, Doylestown, PA (US); Michael S. Kinch, Laytonsville, MD (US); Solomon Langermann, Baltimore, MD (US); Jennifer L. Reed, Clarksburg, MD (US)

Correspondence Address: JONES DAY, 222 EAST 41ST ST, NEW YORK, NY, 10017, US

(21) Appl. No.: 10/823,254

(22) Filed: Apr. 12, 2004

Related U.S. Application Data
(60) Provisional application No. 60/462,024, filed on Apr. 11, 2003.

(51) Int. Cl. A61K 39/395

(52) U.S. Cl. 514/12; 424/144.1

(57) ABSTRACT

The present invention relates to methods and compositions designed for the treatment, management, or prevention of a non-neoplastic hyperproliferative cell or excessive cell accumulation disorders, particularly those involving hyperproliferation of epithelial or endothelial cells. In one embodiment, the methods of the invention comprise the administration of an effective amount of one or more EphA2 agonistic agents that bind to EphA2 and increase EphA2 cytoplasmic tail phosphorylation and/or increase EphA2 autophosphorylation in cells which EphA2 has been agonized. In another embodiment, the methods of the invention comprise the administration of an effective amount of one or more EphA2 agonistic agents that bind to EphA2 and reduce EphA2 activity (other than autophosphorylation). In another embodiment, the methods of the invention comprise administration of an effective amount of one or more EphA2 agonistic agents that bind to EphA2 and decrease a pathology-causing cell phenotype (e.g., a pathology-causing epithelial cell phenotype or a pathology-causing endothelial cell phenotype). In another embodiment, the methods of the invention comprise the administration of an effective amount of one or more EphA2 agonistic agents that are EphA2 antibodies that bind to EphA2 with a very low K_{off} rate. In preferred embodiments, agents of the invention are monoclonal antibodies. The invention also provides pharmaceutical compositions comprising one or more EphA2 agonistic agents of the invention either alone or in combination with one or more other agents useful in therapy for non-neoplastic hyperproliferative cell or excessive cell accumulation disorders.

This application claims priority to U.S. Provisional Application Ser. No. 60/462,024, filed Apr. 11, 2003, which is incorporated herein by reference in its entirety.

### 1. FIELD OF THE INVENTION

The present invention relates to methods and compositions designed for the treatment, management, or prevention of disorders involving non-neoplastic hyperproliferative cells (or excessive cell accumulation), particularly hyperproliferative epithelial and endothelial cells. The methods of the invention comprise the administration of an effective amount of one or more EphA2 agonistic agents that bind EphA2, elicit EphA2 signaling, and thereby reduce EphA2 expression and/or activity. In certain embodiments, the EphA2 agonistic agent of the invention increases EphA2 cytoplasmic tail phosphorylation, increases EphA2 autophosphorylation, reduces EphA2 activity (other than autophosphorylation), decreases a pathology-causing cell phenotype (e.g., a pathology-causing epithelial cell phenotype or a pathology-causing endothelial cell phenotype). In preferred embodiments, the EphA2 agonistic agent is an anti-EphA2 antibody, preferably monoclonal, which preferably has a low K_{off} rate (e.g., K_{off} less than 3×10⁻³ s⁻¹). The invention also provides pharmaceutical compositions comprising one or more EphA2 agonistic agents of the invention either alone or in combination with one or more other agents useful in therapy for a non-neoplastic hyperproliferative cell or excessive cell accumulation disorder. Diagnostic methods and methods for screening for therapeutically useful agents are also provided.

### 2. BACKGROUND OF THE INVENTION

EphA2

EphA2 is a 130 kDa receptor tyrosine kinase that is expressed in adult epithelia, where it is found at low levels and is enriched within sites of cell-cell adhesion (Zantek, et al, Cell Growth & Differentiation 10:629, 1999; R. A. Lindberg, et al., Molecular & Cellular Biology 10: 6316, 1990). This subcellular localization is important because EphA2 binds ligands (known as Ephrin A1 to Ephrin A5) that are anchored to the cell membrane (Eph Nomenclature Committee, 1997, Cell 90:403; Gale, et al., 1997, Cell & Tissue Research 290: 227). The primary consequence of ligand binding is EphA2 autophosphorylation (Lindberg, et al., 1990, supra). However, unlike other receptor tyrosine kinases, EphA2 retains activity in the absence of ligand binding or phosphotyrosine content (Zantek, et al., 1999, supra). Antibodies to EphA2 have been made and proposed to be useful in the treatment of cancer (see e.g., International Patent Publication Nos. WO 01/12840 and WO 01/12172; U.S. Provisional Patent Application Nos. 60/379,322 and 60/379,368; U.S. Pat. No. 5,824,303). Upregulation of EphA2 is induced by deoxycholic acid (DCA) in human colon carcinoma cells in an erkl/2 pathway-dependent manner (L1, et al., 2003, J Cancer Res. Clin. Oncol., 129:703).

Asthma

Asthma is a disorder characterized by intermittent airway obstruction. In western countries it affects 15% of the pediatric population and 7.5% of the adult population (Strachan et al., 1994, Arch. Dis. Child 70:174-178). Most asthma in children and young adults is initiated by IgE mediated allergy (atopy) to inhaled allergens such as house dust mite and cat dander allergens. However, not all asthmatics are atopic, and most atopic individuals do not have asthma. Thus, factors in addition to atopy are necessary to induce the disorder (Fraser et al., eds. (1994) Synopsis of Diseases of the Chest. W B Saunders Company, Philadelphia: 635-53; Djukanovic et al., 1990, Am. Rev. Respir. Dis. 142:434-457). Asthma is strongly familial, and is due to the interaction between genetic and environmental factors. The genetic factors are thought to be variants of normal genes (“polymorphisms”) which alter their function to predispose to asthma.

Asthma may be identified by recurrent wheeze and intermittent air flow limitation. An asthmatic tendency may be quantified by the measurement of bronchial hyper-responsiveness in which an individual's dose-response curve to a broncho-constrictor such as histamine or methacholine is constructed. The curve is commonly summarized by the dose which results in a 20% fall in air flow (PD20) or the slope of the curve between the initial air flow measurement and the last dose given (slope).

In the atopic response, IgE is produced by B-cells in response to allergen stimulation. These antibodies coat mast cells by binding to the high affinity receptor for IgE and initiate a series of cellular events leading to the destabilization of the cell membrane and release of inflammatory mediators. This results in mucosal inflammation, wheezing, coughing, sneezing and nasal blockage.

Atopy can be diagnosed by (i) a positive skin prick test in response to a common allergen; (ii) detecting the presence of specific serum IgE for allergen; or (iii) by detecting elevation of total serum IgE.

COPD

Chronic obstructive pulmonary disease (COPD) is an umbrella term frequently used to describe two conditions of fixed airways disorders, chronic bronchitis and emphysema. Chronic bronchitis and emphysema are most commonly caused by smoking; approximately 90% of patients with COPD are or were smokers. Although approximately 50% of smokers develop chronic bronchitis, only 15% of smokers develop disabling airflow obstruction. Certain animals, particularly horses, suffer from COPD as well.

The airflow obstruction associated with COPD is progressive, may be accompanied by airway hyperactivity, and may be partially reversible. Non-specific airway hyper-responsiveness may also play a role in the development of COPD and may be predictive of an accelerated rate of decline in lung function.

COPD is a significant cause of death and disability. It is currently the fourth leading cause of death in the United States and Europe. Treatment guidelines advocate early detection and implementation of smoking cessation programs to help reduce morbidity and mortality due to the disorder. However, early detection and diagnosis has been difficult for a number of reasons. COPD takes years to develop and acute episodes of bronchitis often are not recognized by the general practitioner as early signs of COPD. Many patients exhibit features of more than one disorder (e.g., chronic bronchitis or asthmatic bronchitis) making precise diagnosis a challenge, particularly early in the etiology of the disorder. Also, many patients do not seek medical help until they are experiencing more severe symptoms associated with reduced lung function, such as dyspnea, persistent cough, and sputum production. As a consequence, the vast majority of patients are not diagnosed or treated until they are in a more advanced stage of the disorder.

Mucin

Mucins are a family of glycoproteins secreted by the epithelial cells including those at the respiratory, gastrointestinal and female reproductive tracts. Mucins are responsible for the viscoelastic properties of mucus (Thornton, et al., 1997, J. Biol. Chem., 272:9561-9566). Nine mucin genes are known to be expressed in man: MUC 1, MUC 2, MUC 3, MUC 4, MUC 5AC, MUC 5B, MUC 6, MUC 7 and MUC 8 (Bobek et al., 1993, J. Biol. Chem. 268:20563-9; Dusseyn et al., 1997, J. Biol. Chem. 272:3168-78; Gendler et al., 1991, Am. Rev. Resp. Dis. 144:S42-S47; Gum et al., 1989, J. Biol. Chem. 264:6480-6487; Gum et al., 1990, Biochem. Biophys. Res. Comm. 171:407-415; Lesuffleur et al., 1995, J. Biol. Chem. 270:13665-13673; Meerzaman et al., 1994, J. Biol. Chem. 269:12932-12939; Porchet et al., 1991, Biochem. Biophys. Res. Comm. 175:414-422; Shankar et al., 1994, Biochem. J. 300:295-298; Toribara et al., 1997, J. Biol. Chem. 272:16398-403). Many airway disorders such chronic bronchitis, chronic obstructive pulmonary disease, bronchietactis, asthma, cystic fibrosis and bacterial infections are characterized by mucin overproduction (Prescott et al., Eur. Respir. J, 1995, 8:1333-1338; Kim et al., Eur. Respir. J., 1997, 10:1438; Steiger et al., 1995, Am. J. Respir. Cell Mol. Biol., 12:307-314). Mucociliary impairment caused by mucin hypersecretion leads to airway mucus plugging which promotes chronic infection, airflow obstruction and sometimes death. For example, chronic obstructive pulmonary disease (COPD), a disorder characterized by slowly progressive and irreversible airflow limitation is a major cause of death in developed countries. The respiratory degradation consists mainly of decreased luminal diameters due to airway wall thickening and increased mucus caused by goblet cell hyperplasia and hypersecretion. Epidermal growth factor (EGF) is known to upregulate epithelial cell proliferation, and mucin production/secretion (Takeyama et al., 1999, PNAS 96:3081-6; Burgel et al., 2001, J. Immunol. 167:5948-54). EGF also causes mucin-secreting cells, such as goblet cells, to proliferate and increase mucin production in airway epithelia (Lee et al., 2000, Am. J. Physiol. Lung Cell. Mol. Physiol. 278:L185-92; Takeyama et al., 2001, Am. J. Respir. Crit. Care. Med. 163:511-6; Burgel et al., 2000, J. Allergy Clin. Immunol. 106:705-12). Historically, mucus hypersecretion has been treated in two ways: physical methods to increase clearance and mucolytic agents. Neither approach has yielded significant benefit to the patient or reduced mucus obstruction. Therefore, it would be desirable to have methods for reducing mucin production and treating the disorders associated with mucin hypersecretion.

Fibrosis

Progressive fibrosis of liver, kidney, lungs, and other viscera often results in organ failure leading to death or the need for transplantation. These diseases affect millions in the United States and worldwide. For example, hepatic fibrosis is the leading non-malignant gastrointestinal cause of death in the United States. Moreover, it has been increasingly recognized that progression of fibrosis is the single most important determinant of morbidity and mortality in patients with chronic liver disease (Poynard, T. P. et al., 1997, Lancet 349:825-832). Fibrosis is characterized by excessive deposition of matrix components. This leads to destruction of normal tissue architecture and compromised tissue function.

Pulmonary fibrosis can be caused by damaging agents and is associated with hypersensitivity pneumonitis and a strong inflammatory response. Idiopathic pulmonary fibrosis (IPF) is associated with desquamative interstitial pneumonitis (DIP), characterized by mononuclear cells in the alveoli and little cellular infiltrate in the interstitium. IPF is also associated with usual interstitial pneumonitis (UIP), characterized by patchy interstitial infiltrate and thickening of alveolar walls. The histology of pulmonary fibrosis includes alveolar wall thickening (which may include a “honeycombing” effect), metaplastic epithelium, and changes to fibroblasts including proliferation/ECM accumulation, myofibroblast differentiation, and fibroblastic foci.

Wound healing and fibrosis follow similar pathways. Both involve damage to the epithelium, followed by proliferation and differentiation of fibroblasts and ECM deposition. Both are mediated by cell signaling messengers such as TGFβ and PDGF. In wound healing, tissue regeneration ceases once the wound is healed; however, in fibrosis, cell growth does not stop, leading to continued ECM deposition and a lack of protease activity. Bleomycin induces lung epithelial cell death, followed by acute neutrophilic influx, subsequent chronic inflammation, and parenchymal fibrosis within 4 weeks of administration to susceptible strains of mice. Bleomycin-treated lung epithelial cells as a model for lung fibrosis replicates key pathologic features of human IPF, including fibroproliferation within the lung parenchyma and other pathologic conditions (Dunsmore and Shapiro, 2004, J. Clin. Invest. 113:180-182). Fibrosis induced by bleomycin can be prevented by addition of soluble Fas, which blocks Fas-mediated apoptosis (Kuwano, et al., 1999, J. Clin. Invest. 104:13-9). Fas-mediated apoptosis in the epithelium of IPF tissue is characterized by an increase in Fas and/or Fas ligand. Correspondingly, factors such as soluble Fas that cause a decrease in epithelial apoptosis also show protection against fibrosis.

Asbestosis (interstitial fibrosis) is defined as diffuse lung fibrosis due to the inhalation of asbestos fibers. C. A. Staples, Radiologic Clinics of North America, 30 (6): 1195, 1992. It is one of the major causes of occupationally related lung damage. Merck Index, 1999 (17^{th} ed.), 622. Asbestosis characteristically occurs following a latent period of 15-20 years, with a progression of disease even after exposure has ceased, but rarely occurs in the absence of pleural plaques. C. Peacock, Clinical Radiology, 55: 425, 2000. Fibrosis first arises in and around the respiratory bronchioles, predominating in the subpleural portions of the lung in the lower lobes, and then progresses centrally. C. A. Staples, Radiologic Clinics of North America, 30 (6): 1195, 1992. Asbestosis may cause an insidious onset of progressive dyspnea in addition to a dry cough. The incidence of lung cancer is increased in smokers with asbestosis, and a dose-response relationship has been observed. Merck Index, 1999 (17^{th} ed.), 623.

Additional therapeutics are needed to diagnose and treat fibrotic diseases. For example, no treatments for fibrotic lung diseases such as asbestosis are known to be effective.

Restenosis

Vascular interventions, including angioplasty, stenting, atherectomy and grafting are often complicated by undesirable effects. Exposure to a medical device which is implanted or inserted into the body of a patient can cause the body tissue to exhibit adverse physiological reactions. For instance, the insertion or implantation of certain catheters or stents can lead to the formation of emboli or clots in blood vessels. Other adverse reactions to vascular intervention include endothelial cell proliferation which can lead to hyperplasia, restenosis, i.e. the re-occlusion of the artery, occlusion of blood vessels, platelet aggregation, and calcification. Treatment of restenosis often involves a second angioplasty or bypass surgery. In particular, restenosis may be due to endothelial cell injury caused by the vascular intervention in treating a restenosis.

Angioplasty involves insertion of a balloon catheter into an artery at the site of a partially obstructive atherosclerotic lesion. Inflation of the balloon is intended to rupture the intima and dilate the obstruction. About 20 to 30% of obstructions reocclude in just a few days or weeks (Eltchaninoff et al., 1998, J. Am Coll. Cardiol. 32: 980-984). Use of stents reduces the re-occlusion rate, however a significant percentage continues to result in restenosis. The rate of restenosis after angioplasty is dependent upon a number of factors including the length of the plaque. Stenosis rates vary from 10% to 35% depending the risk factors present. Further, repeat angiography one year later reveals an apparently normal lumen in only about 30% of vessels having undergone the procedure.

Restenosis is caused by an accumulation of extracellular matrix containing collagen and proteoglycans in association with smooth muscle cells which is found in both the atheroma and the arterial hyperplastic lesion after balloon injury or clinical angioplasty. Some of the delay in luminal narrowing with respect to smooth muscle cell proliferation may result from the continuing elaboration of matrix materials by neointimal smooth muscle cells. Various mediators may alter matrix synthesis by smooth muscle cells in vivo.

Neointimal Hyperplasia

Neointimal hyperplasia is the pathological process that underlies graft atherosclerosis, stenosis, and the majority of vascular graft occlusion. Neointimal hyperplasia is commonly seen after various forms of vascular injury and a major component of the vein graft's response to harvest and surgical implantation into high-pressure arterial circulation.

Smooth muscle cells in the middle layer (i.e. media layer) of the vessel wall become activated, divide, proliferate and migrate into the inner layer (i.e. intima layer). The resulting abnormal neointimal cells express pro-inflammatory molecules, including cytokines, chemokines and adhesion molecules that further trigger a cascade of events that lead to occlusive neointimal disease and eventually graft failure.

The proliferation of smooth muscle cells is a critical event in the neointimal hyperplastic response. Using a variety of approaches, studies have clearly demonstrated that blockade of smooth muscle cell proliferation resulted in preservation of normal vessel phenotype and function, causing the reduction of neointimal hyperplasia and graft failure.

Existing treatments for the indications discussed above is inadequate, thus, there exists a need for improved treatments for the above indications.

Citation or discussion of a reference herein shall not be construed as an admission that such is prior art to the present invention.

### 3. SUMMARY OF THE INVENTION

The present inventors have found that EGF causes an increase in EphA2 expression at the level of both protein and mRNA expression. Without being bound by a particular mechanism, the direct effect of EGF-stimulated EphA2 expression, and thus increased EphA2 activity, may be responsible for the phenotypic changes in epithelial and endothelial cells in the presence of EGF.

The present inventors have found that agents that agonize EphA2, i.e., elicit EphA2 autophosphorylation, actually decrease EphA2 expression. Although not intending to be bound by any mechanism of action, agonistic antibodies may repress hyperproliferation by inducing EphA2 autophosphorylation, thereby causing subsequent EphA2 degradation to down-regulate expression. Thus, in one embodiment, the EphA2 agonistic agents of the invention increase cytoplasmic tail phosphorylation of EphA2.

In addition, hyperproliferating cells or excessive cell accumulation in a subject suffering from a non-neoplastic hyperproliferative cell or excessive cell accumulation disorder exhibit phenotypic traits that differ from those of cells in a unaffected subject. For example, in hyperproliferative epithelial cell respiratory disorders, EphA2-expressing non-neoplastic airway epithelial cells from affected subjects demonstrate increased mucin secretion, increased differentiation into a mucin-secreting cell (e.g., goblet cell), increased secretion of inflammatory factors, as well as hyperproliferation or excessive cell accumulation. In other hyperproliferative endothelial or epithelial cell disorders, EphA2-expressing endothelial or epithelial cells from affected subjects demonstrate increased cell migration, increased cell volume, increased secretion of extracellular matrix molecules (e.g., collagens, proteoglycans, fibronectin, etc.), increased secretion of matrix metalloproteinases (e.g., gelatinases, collagenases, and stromelysins) and/or hyperproliferation.

Accordingly, the invention also provides EphA2 agonistic agents of the invention that inhibit one or more pathology-causing cell phenotypes. Exposing hyperproliferating or accumulating cells in a patient suffering from a non-neoplastic hyperproliferative disorder (e.g., a hyperproliferative epithelial cell disorder, such as asthma, COPD, lung fibrosis, asbestosis, IPF, DIP, UIP, kidney fibrosis, liver fibrosis, other fibroses, bronchial hyper responsiveness, psoriasis, seborrheic dermatitis, cystic fibrosis, or a hyperproliferative endothelial cell disorder, such as restenosis, hyperproliferative vascular disease, Behcet's Syndrome, atherosclerosis, and macular degeneration, or a hyperproliferative fibroblast cell disorder) to such EphA2 agonistic agents that reduce one or more pathology-causing cell phenotypes prevents or decreases the cells' ability to cause symptoms of the hyperproliferative disorder. Furthermore, in certain embodiments, the addition of such EphA2 agonistic agents that reduce one or more pathology-causing cell phenotypes causes the hyperproliferating cells or excessive cell accumulation to slow or stop proliferating or causes a reduction or elimination of the number of cells, i.e., leads to killing of hyperproliferative cells, for example through necrosis or apoptosis. In a specific embodiment, the disease or disorder involves pre-malignant cells, such as hyperplasia, metaplasia or dysplasia.

In one embodiment, the non-neoplastic hyperproliferative disorder is not asthma. In another embodiment, the non-neoplastic hyperproliferative disorder is not COPD. In another embodiment, the non-neoplastic hyperproliferative disorder is not psoriasis. In another embodiment, the non-neoplastic hyperproliferative disorder is not lung fibrosis or other fibroses. In another embodiment, the non-neoplastic hyperproliferative disorder is not restenosis.

The present invention provides for the screening and identification of agents that bind to EphA2 and are EphA2 agonists and/or decrease EphA2 activity and/or inhibit a pathology-causing cell phenotype. The EphA2 agonistic agent can be an antibody, preferably a monoclonal antibody, which may have a low K_{off} rate (e.g., K_{off} less than 3×10⁻³ s⁻¹). In one embodiment, the antibodies used in the methods of the invention are Eph099B-102.147, Eph099B-208.261, Eph099B-210.248, B233, EA2 or EA5. In an even more preferred embodiment, the antibodies used in the methods of the invention are human or humanized Eph099B-102.147, Eph099B-208.261, Eph099B-210.248, B233, EA2, or EA5.

Accordingly, the present invention relates to pharmaceutical compositions and prophylactic and therapeutic regimens designed to prevent, treat, or manage a disorder associated with overexpression of EphA2 and/or non-neoplastic hyperproliferation, particularly of epithelial or endothelial cells, in a subject comprising administering one or more EphA2 agonistic agents of the invention that bind to EphA2 and increase EphA2 cytoplasmic tail phosphorylation, increase EphA2 autophosphorylation, reduce EphA2 expression and/or activity (other than autophosphorylation), and/or decrease a pathology-causing cell phenotype (e.g., a pathology-causing epithelial cell phenotype or a pathology-causing endothelial cell phenotype).

In preferred embodiments, the EphA2 agonistic agent decreases the secretion of mucin, the differentiation of EphA2-expressing cells into mucin-secreting cells, secretion of inflammatory factors, non-neoplastic cell hyperproliferation, cell migration (excluding, in preferred embodiments, metastasis), cell volume and/or secretion of extracellular matrix molecules or matrix metalloproteinases, for example, fibronectin. In a preferred embodiment, the methods of the invention are used to prevent, treat, or manage symptoms of a non-neoplastic hyperproliferative cell or excessive cell accumulation disorder, particularly those disorders displaying (and, to some extent, caused or aggravated by) hyperproliferating and/or accumulating epithelial or endothelial cells or hyperproliferating fibroblasts. The agents of the invention can be administered in combination with one or more other non-neoplastic hyperproliferative cell or excessive cell accumulation disorder therapies. In particular, the present invention provides methods of preventing, treating, or managing a non-neoplastic hyperproliferative cell or excessive cell accumulation disorder in a subject comprising administering to said subject a therapeutically or prophylactically effective amount of one or more EphA2 agonistic agents of the invention in combination with the administration of a therapeutically or prophylactically effective amount of one or more other non-neoplastic hyperproliferative cell or excessive cell accumulation disorder therapies other than the administration of an EphA2 agonistic agent of the invention. In other embodiments, the invention provides methods of treating, preventing, or managing a non-neoplastic hyperproliferative cell or excessive cell accumulation disorder by administering immunomodulatory agents, EphA4 agonistic agents, or anti-viral agents in combination with EphA2 agonistic agents of the invention. In preferred embodiments, respiratory disorders, e.g., asthma, COPD, lung fibrosis, bronchial hyper responsiveness, cystic fibrosis etc., associated with respiratory infection are treated, managed, or prevented with one or more EphA2 agonistic agents and one or more anti-respiratory agents, e.g., anti-RSV antibodies (e.g., palivizumab or A4B4, see PCT Application Serial no. PCT/US01/44807, filed Nov. 28, 2001), anti-HMPV antibodies and/or anti-PIV antibodies.

The methods and compositions of the invention are useful not only in untreated patients but are also useful in the treatment of patients partially or completely refractory to current standard and experimental non-neoplastic hyperproliferative cell or excessive cell accumulation disorder therapies.

In addition, the present invention provides methods of screening for EphA2 agonistic agents of the invention. In particular, candidate EphA2 agonistic agents may be screened for binding to EphA2 and increase EphA2 cytoplasmic tail phosphorylation, increase EphA2 autophosphorylation, or reduce EphA2 activity (other than autophosphorylation), increase EphA2 degradation, reduce a pathology-causing cell phenotype. In embodiments where the EphA2 agonistic agents of the invention are antibodies, the EphA2 antibodies may be screened using antibody binding kinetic assays well known in the art (e.g. BIACORE assays) to identify antibodies having a low K_{off} rate (e.g., K_{off} less than 3×10⁻³ s⁻¹).

In another embodiment, to identify a pathology-causing cell phenotype inhibiting EphA2 agonistic agent, candidate agents may be screened for the ability to prevent or reduce secretion of mucin, differentiation of an epithelial cell into a mucin-secreting cell, secretion of inflammatory factors, non-neoplastic hyperproliferation, non-neoplastic cell migration, increased cell volume, and/or secretion of extracellular matrix molecules or matrix metalloproteinases.

The invention further provides diagnostic methods using the EphA2 antibodies of the invention to evaluate the efficacy of treatment of a non-neoplastic hyperproliferative cell disorder, wherein the treatment monitored can be either EphA2-based or not EphA2-based. In general, increased EphA2 expression is associated with increased symptoms of a non-neoplastic hyperproliferative cell or excessive cell accumulation disorder. Accordingly, a reduction in EphA2 expression (e.g., decreased EphA2 mRNA or polypeptide expression) with a particular treatment indicates that the treatment is ameliorating the symptoms of a non-neoplastic hyperproliferative cell or excessive cell accumulation disorder. The diagnostic methods of the invention may also be used to prognose or predict a non-neoplastic hyperproliferative cell or excessive cell accumulation disorder. The antibodies of the invention may also be used for immunohistochemical analyses of frozen or fixed cells or tissue assays.

In another embodiment, kits comprising the pharmaceutical compositions or diagnostic reagents of the invention are provided.

3.1. Definitions

As used herein, the term “agent” refers to a molecule that has a desired biological effect. Agents include, but are not limited to, proteinaceous molecules, including, but not limited to, peptides, polypeptides, proteins, including post-translationally modified proteins, antibodies etc.; or small molecules (less than 1000 daltons), inorganic or organic compounds; or nucleic acid molecules including, but not limited to, double-stranded or single-stranded DNA, or double-stranded or single-stranded RNA, as well as triple helix nucleic acid molecules. Agents can be derived from any known organism (including, but not limited to, animals, plants, bacteria, fungi, and protista, or viruses) or from a library of synthetic molecules. Agents that are EphA2 agonistic agents bind to EphA2 and reduce EphA2 expression and/or activity (other than autophosphorylation) and/or inhibits a pathology-causing cell phenotype (e.g., decreases the secretion of mucin, the differentiation of EphA2-expressing cells into a mucin-secreting cell, secretion of inflammatory factors, cell hyperproliferation, cell migration, cell volume, secretion of extracellular matrix molecules or matrix metalloproteinases). In preferred embodiments, the EphA2 agonistic agent is an antibody, preferably a monoclonal antibody, which preferably has a low K_{off} rate (e.g., K_{off} less than 3×10⁻³ s⁻¹). An antibody that is an EphA2 agonistic agent may or may not bind to an epitiope that is in the EphA2 ligand binding site.

As used herein, the term “antibodies or fragments thereof that immunospecifically bind to EphA2” refers to antibodies or fragments thereof that specifically bind to an EphA2 polypeptide or a fragment of an EphA2 polypeptide and do not specifically bind to other non-EphA2 polypeptides. Preferably, antibodies or fragments that immunospecifically bind to an EphA2 polypeptide or fragment thereof do not cross-react with other antigens. Antibodies or fragments that immunospecifically bind to an EphA2 polypeptide can be identified, for example, by immunoassays or other techniques known to those of skill in the art. Antibodies of the invention include, but are not limited to, synthetic antibodies, monoclonal antibodies, recombinantly produced antibodies, multispecific antibodies (including bi-specific), human antibodies (e.g., monospecific, bi-specific, etc.), humanized antibodies, chimeric antibodies, synthetic antibodies, intrabodies, single-chain Fvs (scFv) (e.g., monospecific, bi-specific, etc.), Fab fragments, F(ab′) fragments, disulfide-linked Fvs (sdFv), and anti-idiotypic (anti-Id) antibodies, intrabodies, and epitope-binding fragments of any of the above. In particular, antibodies of the present invention include immunoglobulin molecules and immunologically active portions of immunoglobulin molecules, i.e., molecules that contain an antigen binding site that immunospecifically binds to an EphA2 antigen (e.g., one or more complementarity determining regions (CDRs) of an anti-EphA2 antibody). Preferably agonistic antibodies or fragments that immunospecifically bind to an EphA2 polypeptide or fragment thereof only agonize EphA2 and do not significantly agonize other activities.

As used herein, the term “neoplastic” refers to a disease involving cells that have the potential to metastasize to distal sites and exhibit phenotypic traits that differ from those of non-neoplastic cells, for example, formation of colonies in a three-dimensional substrate such as soft agar or the formation of tubular networks or weblike matrices in a three-dimensional basement membrane or extracellular matrix preparation, such as MATRIGEL™. Non-neoplastic cells do not form colonies in soft agar and form distinct sphere-like structures in three-dimensional basement membrane or extracellular matrix preparations. Neoplastic cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. Thus, “non-neoplastic” means that the condition, disease, or disorder does not involve cancer cells.

As used herein, the term “derivative” refers to a polypeptide that comprises an amino acid sequence of an EphA2 polypeptide, a fragment of an EphA2 polypeptide, an antibody that immunospecifically binds to an EphA2 polypeptide, or an antibody fragment that immunospecifically binds to an EphA2 polypeptide which has been altered by the introduction of amino acid residue substitutions, deletions or additions. The term “derivative” as used herein also refers to an EphA2 polypeptide, a fragment of an EphA2 polypeptide, an antibody that immunospecifically binds to an EphA2 polypeptide, or an antibody fragment that immunospecifically binds to an EphA2 polypeptide which has been modified, i.e, by the covalent attachment of any type of molecule to the polypeptide. For example, but not by way of limitation, an EphA2 polypeptide, a fragment of an EphA2 polypeptide, an antibody, or antibody fragment may be modified, e.g., by glycosylation, acetylation, pegylation, phosphorylation, amidation, derivatization by known protecting/blocking groups, proteolytic cleavage, linkage to a cellular ligand or other protein, etc. A derivative of an EphA2 polypeptide, a fragment of an EphA2 polypeptide, an antibody, or antibody fragment may be modified by chemical modifications using techniques known to those of skill in the art, including, but not limited to specific chemical cleavage, acetylation, formylation, metabolic synthesis of tunicamycin, etc. Further, a derivative of an EphA2 polypeptide, a fragment of an EphA2 polypeptide, an antibody, or antibody fragment may contain one or more non-classical amino acids. In one embodiment, a polypeptide derivative possesses a similar or identical function as an EphA2 polypeptide, a fragment of an EphA2 polypeptide, an antibody, or antibody fragment described herein. In another embodiment, a derivative of EphA2 polypeptide, a fragment of an EphA2 polypeptide, an antibody, or antibody fragment has an altered activity when compared to an unaltered polypeptide. For example, a derivative antibody or fragment thereof can bind to its epitope more tightly or be more resistant to proteolysis.

As used herein, the term “EphA2 agonist” refers to any agent, including a protein, polypeptide, peptide, antibody, antibody fragment, large molecule, or small molecule (less than 1000 daltons), that causes increased phosphorylation and subsequent degradation of EphA2 protein. EphA2 agonistic agents that are antibodies may or may not also have a low K_{off} rate.

As used herein, the term “epitope” refers to a portion of an EphA2 polypeptide having antigenic or immunogenic activity in an animal, preferably in a mammal, and most preferably in a human. An epitope having immunogenic activity is a portion of an EphA2 polypeptide that elicits an antibody response in an animal. An epitope having antigenic activity is a portion of an EphA2 polypeptide to which an antibody immunospecifically binds as determined by any method well known in the art, for example, by immunoassays. Antigenic epitopes need not necessarily be immunogenic.

As used herein, the term “fragment” includes a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of an EphA2 polypeptide or an antibody that immunospecifically binds to an EphA2 polypeptide. Preferably, antibody fragments are epitope-binding fragments.

As used herein, the term “human infant” refers to a human less than 24 months, preferably less than 16 months, less than 12 months, less than 6 months, less than 3 months, less than 2 months, or less than 1 month of age. A human infant born prematurely refers to a human born at less than 40 weeks gestational age, less than 35 weeks gestational age. In specific embodiments, the prematurely born human infant is of between 30-35 weeks of gestational age. In specific embodiments, the prematurely born human infant is of between 35-38 weeks of gestational age. In certain embodiments, the prematurely born infant is of 38 weeks gestational age, preferably, the infant is of less than 38 weeks gestational age.

As used herein, the term “humanized antibody” refers to forms of non-human (e.g., murine) antibodies, preferably chimeric antibodies, which contain minimal sequence derived from non-human immunoglobulin. For the most part, humanized antibodies are human immunoglobulins (recipient antibody) in which hypervariable region or complementarity determining (CDR) residues of the recipient are replaced by hypervariable region residues or CDR residues from an antibody from a non-human species (donor antibody), such as mouse, rat, rabbit or non-human primate, having the desired specificity, affinity, and capacity. In some instances, one or more Framework Region (FR) residues of the human immunoglobulin are replaced by corresponding non-human residues or other residues based upon structural modeling, e.g., to improve affinity of the humanized antibody. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., 1986, Nature 321:522-525; Reichmann et al., 1988, Nature 332:323-329; Presta, 1992, Curr. Op. Struct. Biol. 2:593-596, Queen et al., U.S. Pat. No. 5,585,089.

As used herein, the terms “hyperproliferative cell disorder” and “excessive cell accumulation disorder” refers to a disorder that is not neo-plastic, in which cellular hyperproliferation or any form of excessive cell accumulation causes or contributes to the pathological state or symptoms of the disorder. In some embodiments, the hyperproliferative cell or excessive cell accumulation disorder is characterized by hyperproliferating epithelial cells. Hyperproliferative epithelial cell disorders include, but are not limited to, asthma, COPD, lung fibrosis, bronchial hyper responsiveness, psoriasis, seborrheic dermatitis, and cystic fibrosis. In other embodiments, the hyperproliferative cell or excessive cell accumulation disorder is characterized by hyperproliferating endothelial cells. Hyperproliferative endothelial cell disorders include, but are not limited to restenosis, hyperproliferative vascular disease, Behcet's Syndrome, atherosclerosis, and macular degeneration. In other embodiments, the hyperproliferative cell or excessive cell accumulation disorder is characterized by hyperproliferating fibroblasts.

As used herein, the term “hypervariable region” refers to the amino acid residues of an antibody which are responsible for antigen binding. The hypervariable region comprises amino acid residues from a “Complementarity Determining Region” or “CDR” (i.e. residues 24-34 (L1), 50-56 (L2) and 89-97 (L3) in the light chain variable domain and 31-35 (HI), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined.

As used herein, the terms “immunomodulatory agent”, refer to an agent that modulates a subject's immune system. In particular, an immunomodulatory agent is an agent that alters the ability of a subject's immune system to respond to one or more foreign antigens. In a specific embodiment, an immunomodulatory agent is an agent that shifts one aspect of a subject's immune response. In a preferred embodiment of the invention, an immunomodulatory agent is an agent that inhibits or reduces a subject's immune response (i.e., an immunosuppressant agent). Preferably, an immunomodulatory agent that inhibits or reduces a subject's immune response inhibits or reduces the ability of a subject's immune system to respond to one or more foreign antigens. In certain embodiments, antibodies that immunospecifically bind IL-9 are immunomodulatory agents.

As used herein, the term “in combination” refers to the use of more than one prophylactic and/or therapeutic agents. The use of the term “in combination” does not restrict the order in which prophylactic and/or therapeutic agents are administered to a subject with a hyperproliferative epithelial or endothelial cell disorder or disorder associated with excessive cell accumulation. A first prophylactic or therapeutic agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject which had, has, or is susceptible to a hyperproliferative epithelial or endothelial cell disorder or disorder associated with excessive cell accumulation. The prophylactic or therapeutic agents are administered to a subject in a sequence and within a time interval such that the agent of the invention can act together with the other agent to provide an increased benefit than if they were administered otherwise. Any additional prophylactic or therapeutic agent can be administered in any order with the other additional prophylactic or therapeutic agents. In certain embodiments, EphA2 agonistic agents of the invention can be administered in combination with immunomodulatory or anti-viral agents.

As used herein, the terms “manage”, “managing” and “management” refer to the beneficial effects that a subject derives from a prophylactic or therapeutic agent, which does not result in a cure of the disorder. In certain embodiments, a subject is administered one or more prophylactic or therapeutic agents to “manage” a disorder so as to prevent the progression or worsening of the disorder.

As used herein, the term “pathology-causing cell phenotype” refers to a function that a hyperproliferating cell performs that causes or contributes to the pathological state of a hyperproliferative disorder. Pathology-causing epithelial cell phenotypes include secretion of mucin, differentiation into a mucin-secreting cell, secretion of inflammatory factors, and hyperproliferation. Pathology-causing endothelial cell phenotypes include increased cell migration (not including metastasis), increased cell volume, secretion of extracellular matrix molecules (e.g., collagen, fibronectin, proteoglycans, etc.) or matrix metalloproteinases (e.g., gelatinases, collagenases, and stromelysins), and hyperproliferation. One or more of these pathology-causing cell phenotypes causes or contributes to symptoms in a patient suffering from a hyperproliferative cell or excessive cell accumulation disorder.

As used herein, the term “potentiate” refers to an improvement in the efficacy of a therapeutic agent at its common or approved dose.

As used herein, the terms “prevent”, “preventing” and “prevention” refer to the prevention of the recurrence, spread or onset of a disorder in a subject resulting from the administration of a prophylactic or therapeutic agent.

As used herein, the term “prophylactic agent” refers to any agent that can be used in the prevention of the spread, onset, or recurrence of a disorder associated with EphA2 overexpression and/or hyperproliferation of cells, particularly, epithelial or endothelial cells. In certain embodiments, the term “prophylactic agent” refers to an EphA2 agonistic agent that decreases EphA2 expression, increases EphA2 cytoplasmic tail phosphorylation, decreases EphA2 activity (other than autophosphorylation), and/or inhibits a pathology-causing cell phenotype. In certain embodiments, the EphA2 prophylactic agent is a monoclonal antibody which may have a low K_{off} rate. In certain embodiments, Eph099B-102.147, Eph099B-208.261, Eph099B-210.248, B233, EA2, EA5, or humanized forms thereof are prophylactic agents. The term “prophylactic agent” can also refer to an agent used in non-EphA2-based therapies to prevent the spread, onset, or recurrence of a hyperproliferative disorder or other therapies useful in the amelioration of symptoms, including, but not limited to, immunomodulatory and/or anti-viral therapies.

As used herein, a “prophylactically effective amount” refers to that amount of the prophylactic agent sufficient to result in the prevention of the spread, onset, or recurrence of a hyperproliferative cell or excessive cell accumulation disorder, particularly those caused by hyperproliferating epithelial or endothelial cells or hyperproliferating fibroblasts. A prophylactically effective amount may refer to the amount of prophylactic agent sufficient to prevent the spread, onset, or recurrence of a hyperproliferative cell or excessive cell accumulation disorder, including but not limited to those predisposed to a hyperproliferative cell or excessive cell accumulation disorder, for example those genetically predisposed or those exposed to tobacco smoke or those infected or previously infected with an upper respiratory tract infection or those who have had angioplasty or those with a history of a hyperproliferative disorder. A prophylactically effective amount may also refer to the amount of the prophylactic agent that provides a prophylactic benefit in the prevention of a hyperproliferative cell or excessive cell accumulation disorder. Further, a prophylactically effective amount with respect to a prophylactic agent of the invention means that amount of prophylactic agent alone, or in combination with other agents, that provides a prophylactic benefit in the prevention of a hyperproliferative cell or excessive cell accumulation disorder. Used in connection with an amount of an EphA2 agonistic agent of the invention, the term can encompass an amount that improves overall prophylaxis or enhances the prophylactic efficacy of or synergies with another prophylactic agent.

A used herein, a “protocol” includes dosing schedules and dosing regimens.

As used herein, the term “refractory” refers to a hyperproliferative cell or excessive cell accumulation disorder that is not responsive to a particular treatment. In a certain embodiment, that a hyperproliferative cell or excessive cell accumulation disorder is refractory to a therapy means that at least some significant portion of the symptoms associated with said disorder are not eliminated or lessened by that therapy. The determination of whether a hyperproliferative cell or excessive cell accumulation disorder is refractory can be made either in vivo or in vitro by any method known in the art for assaying the effectiveness of treatment of a hyperproliferative cell or excessive cell accumulation disorder. In some embodiments, effectiveness of asthma treatment is measured by monitoring the frequency of attacks and lung hyper responsiveness. In other embodiments, effectiveness of COPD treatment is measured by monitoring the number of bacterial infections, patient self evaluation in ability to exercise, and forced expiratory volume per one second or ten seconds (FEV₁ or FEV₁₀).

As used herein, the phrase “side effects” encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky. Examples of side effects include, but are not limited to, nausea, vomiting, anorexia, abdominal cramping, fever, pain, loss of body weight, dehydration, alopecia, dyspnea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physicians' Desk Reference (56^{th} ed., 2002).

As used herein, the terms “single-chain Fv” or “sFv” refer to antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 1113, Rosenburg and Moore eds. Springer-Verlag, New York, pp. 269-315 (1994).

As used herein, the terms “subject” and “patient” are used interchangeably. As used herein, a subject is preferably a mammal such as a non-primate (e.g., cows, pigs, horses, cats, dogs, rats, etc.) and a primate (e.g., monkey and human), most preferably a human.

As used herein, the term “therapy” refers to any protocol, method and/or agent that can be used in the prevention, treatment, or management of a disorder associated with EphA2 overexpression and/or cell hyperproliferation, particularly of epithelial or endothelial cells.

As used herein, the term “therapeutic agent” refers to any agent that can be used in the prevention, treatment, or management of a disorder associated with overexpression of EphA2 and/or hyperproliferation, particularly those disorders caused by hyperproliferating epithelial cells or endothelial cells. In certain embodiments, the term “therapeutic agent” refers to an EphA2 agonistic agent that decreases EphA2 expression, increases EphA2 cytoplasmic tail phosphorylation, decreases EphA2 activity (other than autophosphorylation), and/or inhibits a pathology-causing cell phenotype. In certain embodiments, the EphA2 therapeutic agent is a monoclonal antibody which has a low K_{off} rate. In certain embodiments, Eph099B-102.147, Eph099B-208.261, Eph099B-210.248, B233, EA2, or EA5 are therapeutic agents. The term “therapeutic agent” can also refer to an agent used in non-EphA2-based therapies to treat hyperproliferative disorders or other therapies useful in the amelioration of symptoms, including, but not limited to, immunomodulatory and/or anti-viral therapies.

As used herein, a “therapeutic protocol” refers to a regimen of timing and dosing of one or more therapeutic agents.

As used herein, a “therapeutically effective amount” refers to that amount of the therapeutic agent sufficient to treat or manage a disorder associated with EphA2 overexpression and/or hyperproliferation and, preferably, the amount sufficient to eliminate, modify, or control symptoms associated with such a disorder. A therapeutically effective amount may refer to the amount of therapeutic agent sufficient to delay or minimize the onset of the hyperproliferative cell or excessive cell accumulation disorder. A therapeutically effective amount may also refer to the amount of the therapeutic agent that provides a therapeutic benefit in the treatment or management of a hyperproliferative cell or excessive cell accumulation disorder. Further, a therapeutically effective amount with respect to a therapeutic agent of the invention means that amount of therapeutic agent alone, or in combination with other therapies, that provides a therapeutic benefit in the treatment or management of a hyperproliferative cell or excessive cell accumulation disorder. Used in connection with an amount of an EphA2 agonistic agent of the invention, the term can encompass an amount that improves overall therapy, reduces or avoids unwanted effects, or enhances the therapeutic efficacy of or synergies with another therapeutic agent.

As used herein, the terms “treat”, “treating” and “treatment” refer to the eradication, reduction or amelioration of symptoms of a disorder, particularly, the eradication, removal, modification, or control of asthma, COPD, fibrosis, or restenosis that results from the administration of one or more prophylactic or therapeutic agents. In certain embodiments, such terms refer to the minimizing the symptoms associated with asthma, COPD, fibrosis, or restenosis resulting from the administration of one or more prophylactic or therapeutic agents to a subject with such a disorder.

### 4. DESCRIPTION OF THE FIGURES

FIGS. 1A-1B: EGF increases EphA2 expression. HMT-3522 cells, variant S1 (a non-tumorigenic immortalized epithelial cell line) were incubated with EGF. (A) Quantitative PCR analysis demonstrated that EphA2 mRNA levels were increased with EGF treatment as compared to control cells not treated with EGF. (B) Western blot analysis of whole cell lysates with EphA2-specific D7 antibody demonstrated that EphA2 protein levels were increased with EGF treatment as compared to control cells not treated with EGF. The relative mobility of molecular mass standards is shown on the left.

FIGS. 2A-2B: EphA2 expression on lung epithelium in vivo. Lung tissue from BALB/c mice was stained with an EphA2-specific antibody. Both normal mice (A) and RSV-infected mice (B, right panel) showed staining on the epithelial cells of the basal layer. Staining using periodic acid-Schiff (PAS) reagent which stains the mucin produced by goblet cells (B, left panel) was found to be on different cells than EphA2 in lung tissue from RSV-infected mice.

FIG. 3: Kinetic analysis of EphA2 monoclonal antibodies. BIACORE™ assays were used to assay the kinetics of EphA2 monoclonal antibody binding to immobilized EphA2-Fc. Eph099B-208.261 is indicated by a solid line, B233 is indicated by a dotted line, EA2 is indicated by a dashed line, and the negative control is indicated by squares.

FIG. 4: EphA2 antisense can reduce EphA2 protein levels. Monolayers of MDA-MB-231 cells were transfected with 2 μg/ml of EphA2 antisense or inverse antisense (IAS) oligonucleotides at 37° C. for 24 hours. Western blot analysis of whole cell lysates with EphA2-specific D7 antibody confirms that transfection with antisense oligonucleotides decreases EphA2 protein levels. The membranes were stripped and reprobed with paxillin antibodies as a loading control. The relative mobility of molecular mass standards is shown on the left.

FIGS. 5A-5D: The amino acid sequences of VL and VH of Eph099B-208.261 and B233 antibodies. Sequences of the CDRs are indicated.

FIG. 6: Altered Adhesion and Signaling in Transformed Epithelia. Normal epithelia shows stable cell-cell adhesions and weak extracellular matrix (ECM) adhesion, low cellular migration, low cellular proliferation, and low EphA2 levels. However, transformed epithelia shows altered adhesion and signaling more characteristic of tissue regeneration, including weak cell-cell adhesions, increased ECM adhesion, high cellular migration, high cellular proliferation, and high EphA2 levels.

FIG. 7: Upregulation of EphA2 alters adhesion properties of epithelium. Examination of MCF10A mammary epithelial cells by phase-contrast microscopy, or with E-cadherin and Paxillin staining, reveals decreased cell-cell adhesion in EphA2-upregulated cells relative to control cells.

FIG. 8: High Levels of Fibronectin in EphA2-Overexpressing Cells. Western Blot of extracts from MCF10A mammary epithelial cell overexpressing Neo (lane 1) or EphA2 (lane 2) show elevated fibronectin expression with increased EphA2 expression.

FIG. 9: EphA2 Antibodies Induce Fibronectin Degradation. Western Blot of extracts from MDA-MB-231 breast carcinoma cells treated with B13 EphA2 antibodies show decreased EphA2 protein levels and degradation of fibronectin over a 24 hour period relative to paxillin protein levels which remain stable over time.

FIG. 10: Changes in Cellular Morphology and P-Tyr Localization. Microscopy of Beas2B cells stained to reveal phosphorylated tyrosine (P-Tyr) shows P-Tyr in focal adhesions in cells treated for 24 hours with bleomycin relative to untreated control cells.

FIG. 11: Presence of focal adhesions in bleomycin treated cells. Bleomycin-treated Beas2B cells show focal adhesions.

FIG. 12: Bleomycin-damaged epithelium secretes IL-8. Beas-2B cells treated with increasing amounts of bleomycin secrete increasing levels of IL-8 over a 24-hour period.

FIG. 13: Bleomycin-damaged epithelium secretes IL-6. Beas-2B cells treated with increasing amounts of bleomycin secrete increasing levels of IL-6 over a 24-hour period.

FIG. 14: Induction of Apoptosis in bleomycin-treated Beas-2B cells. Fluorescence-activated cell sorter (FACS) analysis of Beas-2B cells shows increased apoptotic events 24 hours after bleomycin treatment relative to untreated control cells.

FIG. 15: FACS Data.

FIG. 16: Bleomycin Increases CD95 (Fas) Expression. FACS analysis of Beas-2B cells shows increased CD95/Fas expression 24 hours after treatment with bleomycin relative to untreated control cells.

FIG. 17: Bleomycin Upregulates EphA2 in Beas-2B Bronchial Epithelium. Western Blot of Beas-2B bronchial epithelial cells shows increased EphA2 expression after 24 hours of treatment with bleomycin, compared to expression levels of paxillin which remain stable.

FIG. 18: Bleomycin Increases EphA2 Surface Expression in Beas-2B Cells. FACS analysis of Beas-2B cells shows increased EphA2 surface expression 24 hours after treatment with bleomycin, relative to untreated control cells.

FIG. 19: Bleomycin Induces EphA2 Overexpression and Functional Alteration. Western Blot of Beas-2B bronchial epithelial cells shows increased EphA2 expression after 24 hours of treatment with bleomycin, indicating upregulation of EphA2, while P-Tyr levels decrease slightly, indicating altered function of EphA2.

### 5. DETAILED DESCRIPTION OF THE INVENTION

EGF was previously known to be associated with hyperproliferative epithelial cell disorders, particularly asthma and COPD (i.e., by increasing proliferation and mucin secretion of airway epithelial cells) and hyperproliferative endothelial cell disorders, particularly restenosis (i.e., by increasing neointimal hyperplasia). The present invention is based, in part, on the inventors' discovery that EGF also causes an increase in EphA2 expression. Without being bound by a particular mechanism, EGF causes the increased expression of EphA2 thereby increasing EphA2 activity which causes the cell phenotypes associated with non-neoplastic hyperproliferative cell or excessive cell accumulation disorders, particularly those characterized by hyperproliferating epithelial or endothelial cells or hyperproliferating fiboblasts.

Reduction of this elevated EphA2 expression and/or activity (other than autophosphorylation) may ameliorate symptoms associated with a non-neoplastic hyperproliferative cell or excessive cell accumulation disorder or hyperproliferative fibroblast cell disorder. Such decreased levels of EphA2 expression and/or activity (other than autophosphorylation) can be achieved by EphA2 agonistic agents of the invention. In particular, EphA2 agonistic agents may cause increased EphA2 cytoplasmic tail phosphorylation, increased EphA2 autophosphorylation, increased EphA2 degradation, reduced EphA2 activity (other than autophosphorylation), and/or reduced pathology-causing cell phenotype. In embodiments where EphA2 agonistic agents of the invention are antibodies, the EphA2 antibodies may have a low K_{off} rate (e.g., K_{off} less than 3×10⁻³ s⁻¹).

Although not intending to be bound by any mechanism of action, this inhibition of EphA2-dependent symptoms is achieved by EphA2 agonistic agents that agonize EphA2 thereby causing EphA2 autophosphorylation which leads to the degradation of EphA2. Pathology is reduced with reduced EphA2 expression and thus reduced EphA2 activity (other than autophosphorylation).

Accordingly, the present invention relates to methods and compositions that provide for the treatment, inhibition, and management of disorders associated with overexpression of EphA2 and/or increased EphA2 activity and/or hyperproliferation of cells, in particular epithelial and endothelial cells. Further compositions and methods of the invention include other types of active ingredients in combination with the EphA2 agonistic agents of the invention.

The present invention also relates to methods for the treatment, inhibition, and management of non-neoplastic hyperproliferative cell or excessive cell accumulation disorders that have become partially or completely refractory to current treatment.

The invention further provides diagnostic methods using the EphA2 antibodies of the invention to evaluate the efficacy of non-neoplastic hyperproliferative cell or excessive cell accumulation disorder treatment, either EphA2-based or not EphA2-based. The diagnostic methods of the invention can also be used to prognose or predict non-neoplastic hyperproliferative cell or excessive cell accumulation disorder severity.

The present invention provides for the screening and identification of agents that bind to EphA2 and are EphA2 agonists and/or increase EphA2 cytoplasmic tail phosphorylation, increase EphA2 autophosphorylation, increase EphA2 degradation, reduce EphA2 activity (other than autophosphorylation), and/or reduce pathology-causing cell phenotype. The EphA2 agonistic agent can be a antibody, preferably monoclonal, which preferably has a low K_{off} rate (e.g., K_{off} less than 3×10⁻³ s⁻¹).

5.1 EphA2 Agonistic Agents

As discussed above, the invention encompasses administration of EphA2 agonists that increase EphA2 cytoplasmic tail phosphorylation, increase EphA2 autophosphorylation, reduce EphA2 activity (other than autophosphorylation), and/or decrease a pathology-causing cell phenotype (e.g., decreases the secretion of mucin, the differentiation of EphA2-expressing cells into a mucin-secreting cell, secretion of inflammatory factors, cell hyperproliferation, cell migration, cell volume and/or secretion of extracellular matrix molecules or matrix metalloproteinases). Such agonistic agents of the invention include, but are not limited to, proteinaceous molecules, including, but not limited to, peptides, polypeptides, proteins, including post-translationally modified proteins, antibodies etc.; or small molecules (less than 1000 daltons), inorganic or organic compounds; or nucleic acid molecules including, but not limited to, double-stranded or single-stranded DNA, or double-stranded or single-stranded RNA, as well as triple helix nucleic acid molecules.

5.2 Polypeptide Agonistic Agents

Methods of the present invention encompasses EphA2 agonistic agents that are polypeptides. In one embodiment, a polypeptide agonistic agent is an EphA2 antibody or fragment thereof that immunospecifically binds EphA2 and agonizes EphA2 (e.g., increases EphA2 cytoplasmic tail phosphorylation, increases EphA2 autophosphorylation, reduces EphA2 activity (other than autophosphorylation), and/or decreases a pathology-causing cell phenotype). In another embodiment, a polypeptide agonistic agent is an EphA2 ligand (e.g., Ephrin A1 including an Ephrin A1-F_{c} fusion protein) or fragment thereof that is capable of binding EphA2 and agonizing EphA2 (e.g., increases EphA2 cytoplasmic tail phosphorylation, increases EphA2 degradation, decreases survival of EphA2 expressing cells, increases EphA2 autophosphorylation, reduces EphA2 activity (other than autophosphorylation), and/or decreases a pathology-causing cell phenotype.

5.2.1 Antibodies as Polypeptide Agonistic Agents

In one embodiment, EphA2 agonistic agents of the invention encompass antibodies (preferably, monoclonal antibodies) or fragments thereof that immunospecifically bind to EphA2 and increase EphA2 cytoplasmic tail phosphorylation, increase EphA2 autophosphorylation, reduce EphA2 activity (other than autophosphorylation), decrease a pathology-causing cell phenotype (e.g., decrease the secretion of mucin, the differentiation of EphA2-expressing cells into a mucin-secreting cell, secretion of inflammatory factors, non-neoplastic cell hyperproliferation, cell migration (other than metastasis), cell volume and/or secretion of extracellular matrix molecules or matrix metalloproteinases) and/or bind EphA2 with a K_{off} of less than 3×10⁻³ s⁻¹. In one embodiment, the antibody binds to the extracellular domain of EphA2 (e.g., at an epitope either within or outside of the EphA2 ligand binding site) and, preferably, also agonize EphA2, e.g., increases EphA2 phosphorylation and, preferably, causes EphA2 degradation. In another embodiment, the antibody binds to EphA2, preferably the extracellular domain of EphA2 and, preferably, also inhibits and, even more preferably, reduces the number of (e.g., by cell killing mechanisms such as necrosis and apoptosis) the hyperproliferating cells or excessive cell accumulation (e.g., epithelial cells, mucin-secreting cells, cells that differentiate into mucin-secreting cells and/or endothelial cells). In other embodiments, the antibodies inhibit or reduce a pathology-causing cell phenotype in the presence of another agent used in non-neoplastic hyperproliferative cell or excessive cell accumulation disorder therapy. In another embodiment, the antibody binds to the extracellular domain of EphA2, preferably with a K_{off} of less than 1×10⁻³ s⁻¹, more preferably less than 3×10⁻³ s⁻¹. In other embodiments, the antibody binds to EphA2 with a K_{off} of less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹ s⁻¹.

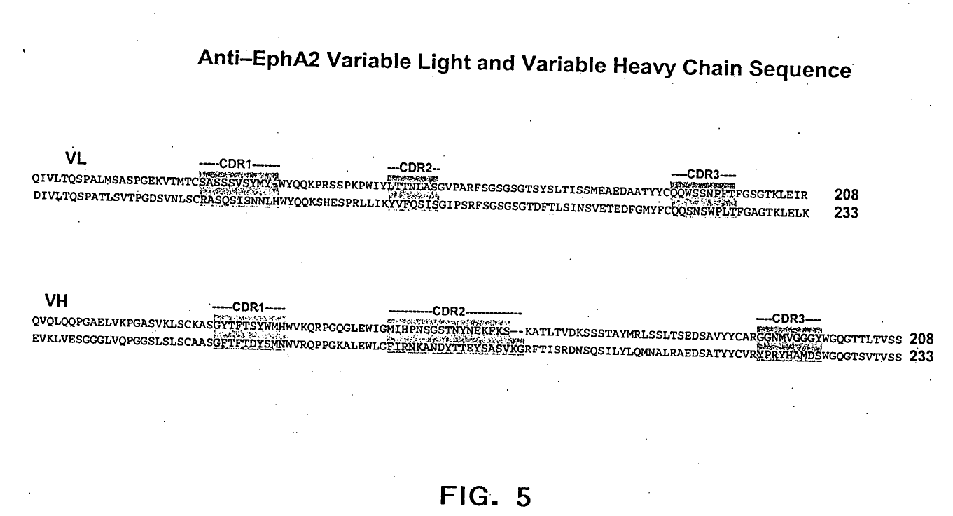

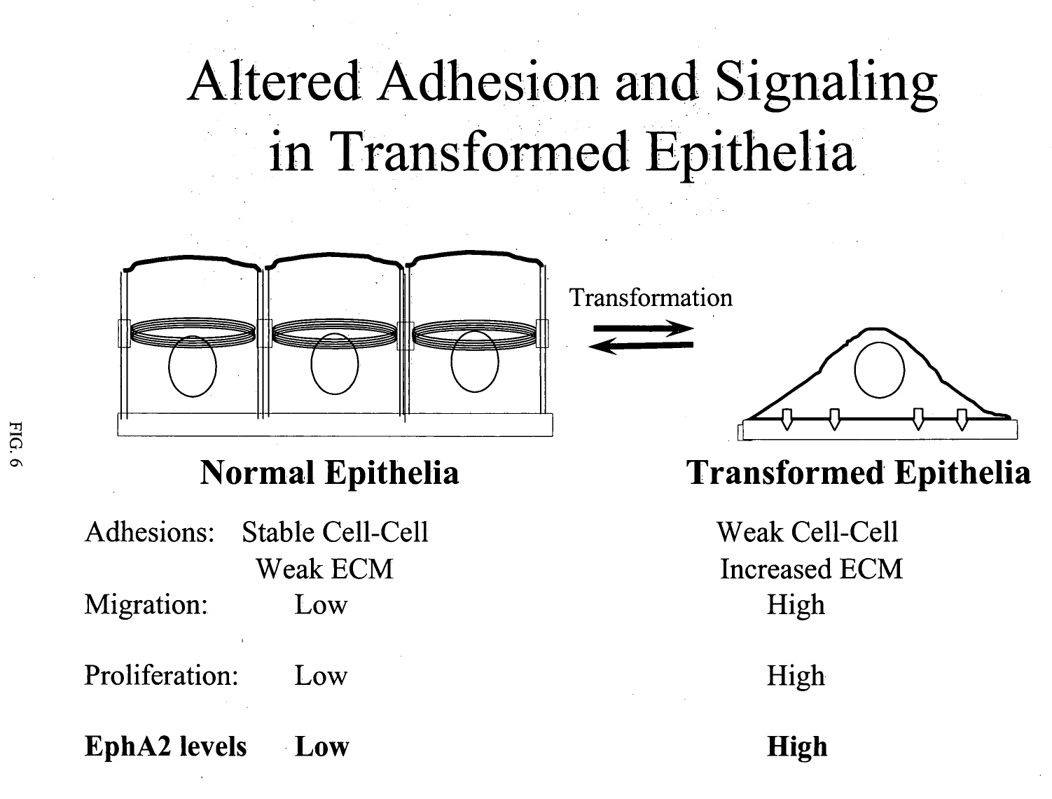

In one embodiment, the antibody is Eph099B-102.147, Eph099B-208.261, Eph099B-210.248, or B233. In another embodiment, the antibodies used in the methods of the invention are EA2 or EA5 (see U.S. patent application Ser. No. 10/463,783 entitled “EphA2 Agonistic Monoclonal Antibodies and Methods of Use Thereof” filed May 12, 2003, which is incorporated by reference in its entirety; hybridomas producing antibodies EA2 (strain EA2.31) and EA5 (strain EA5.12) of the invention have been deposited with the American Type Culture Collection (ATCC, P.O. Box 1549, Manassas, Va. 20108) on May 22, 2002 under the provisions of the Budapest Treaty on the International Recognition of the Deposit of Microorganisms for the Purposes of Patent Procedures, and assigned accession numbers PTA-4380 and PTA-4381, respectively and incorporated by reference. In another embodiment, the antibody used in the methods of the present invention binds to the same epitope as any of Eph099B-102.147, Eph099B-208.261, Eph099B-210.248, B233, EA2, or EA5, or competes with any of Eph099B-102.147, Eph099B-208.261, Eph099B-210.248, B233, EA2, or EA5 for binding to EphA2, e.g. as assayed by ELISA or any other appropriate immunoassay. Hybridomas producing Eph099B-102.147, Eph099B-208.261, and Eph099B-210.248 have been deposited with the American Type Culture Collection (ATCC, P.O. Box 1549, Manassas, Va. 20108) on Aug. 7, 2002 under the provisions of the Budapest Treaty on the International Recognition of the Deposit of Microorganisms for the Purposes of Patent Procedures, and assigned accession numbers PTA-4572, PTA-4573, and PTA-4574, respectively, each of which is incorporated by reference in its entirety. The amino acid sequences of the V_{L} and V_{H} of Eph099B-208.261 and B233 with the CDRs indicated are shown in FIG. 5 (SEQ ID NOs 1-8). In a preferred embodiment, the antibody is human or has been humanized. In another preferred embodiment, the antibody has one or more CDRs of Eph099B-208.261 or B233 in a human framework.

Antibodies of the invention include, but are not limited to, synthetic antibodies, monoclonal antibodies, recombinantly produced antibodies, multispecific antibodies (including bi-specific), human antibodies, humanized antibodies, chimeric antibodies, synthetic antibodies, intrabodies, single-chain Fvs (scFv) (e.g., monospecific, bi-specific, etc.), Fab fragments, F(ab′) fragments, disulfide-linked Fvs (sdFv), and anti-idiotypic (anti-Id) antibodies, intrabodies, and epitope-binding fragments of any of the above. In particular, antibodies used in the methods of the present invention include immunoglobulin molecules and immunologically active portions of immunoglobulin molecules, i.e., molecules that contain an antigen binding site that immunospecifically binds to EphA2 and is an agonist of EphA2 and/or inhibits or reduces a pathology-causing cell phenotype and/or binds EphA2 with a K_{off} of less than 3×10⁻³ s⁻¹. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The present invention encompasses single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Patent Publication Nos. WO 94/04678 and WO 94/25591; U.S. Pat. No. 6,005,079; which are incorporated herein by reference in their entireties). In one embodiment, the present invention provides single domain antibodies comprising two V_{H} domains having the amino acid sequence of any of the V_{H} domains of the EphA2 agonistic antibodies of the invention (e.g., Eph099B-102.147, Eph099B-208.261, Eph099B-210.248, B233, or any other agonistic antibody that increases EphA2 cytoplasmic tail phosphorylation, increases EphA2 autophosphorylation, reduces EphA2 activity (other than autophosphorylation), decreases a pathology-causing cell phenotype, or binds EphA2 with a low K_{off} rate) with modifications such that single domain antibodies are formed. In another embodiment, the present invention also provides single domain antibodies comprising two V_{H} domains comprising one or more of the V_{H} CDRs from any of the EphA2 agonistic antibodies of the invention (e.g., Eph099B-102.147, Eph099B-208.261, Eph099B-210.248, B233, EA2, EA5, or any other agonistic antibody that increases EphA2 cytoplasmic tail phosphorylation, increases EphA2 autophosphorylation, reduces EphA2 activity (other than autophosphorylation), decreases a pathology-causing cell phenotype, or binds EphA2 with a low K_{off} rate). In a preferred embodiment, the present invention provides single domain antibodies comprising two V_{H} domains having the amino acid sequence of any of the V_{H} CDRs from any of Eph099B-102.147, Eph099B-208.261, Eph099B-210.248, or B233.

Antibodies of the invention include EphA2 intrabodies (see Section 5.2.1.1). Antibody agonistic agents of the invention that are intrabodies immunospecifically bind EphA2 and agonize EphA2. In a more specific embodiment, an intrabody of the invention immunospecifically binds to the intracellular domain of EphA2 and causes EphA2 degradation. In another specific embodiment, the intrabody binds to the intracellular domain of EphA2 and decreases and/or slows cell proliferation, growth and/or survival of an EphA2-expressing cell. In another specific embodiment, the intrabody binds to the intracellular domain of EphA2 and maintains/reconstitutes the integrity of an epithelial cell layer.

The antibodies used in the methods of the invention may be from any animal origin including birds and mammals (e.g., human, murine, donkey, sheep, rabbit, goat, guinea pig, camel, horse, or chicken). In a most preferred embodiment, the antibody is human or has been humanized. As used herein, “human” antibodies include antibodies having the amino acid sequence of a human immunoglobulin and include antibodies isolated from human immunoglobulin libraries or from mice that express antibodies from human genes.

The antibodies used in the methods of the present invention may be monospecific, bispecific, trispecific or of greater multispecificity. Multi specific antibodies may immunospecifically bind to different epitopes of an EphA2 polypeptide or may immunospecifically bind to both an EphA2 polypeptide as well a heterologous epitope, such as a heterologous polypeptide or solid support material. See, e.g., International Patent Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

5.2.1.1 Intrabodies

In certain embodiments, the antibody to be used with the invention binds to an intracellular epitope, i.e., is an intrabody. An intrabody comprises at least a portion of an antibody that is capable of immunospecifically binding an antigen and preferably does not contain sequences coding for its secretion. Such antibodies will bind antigen intracellularly. In one embodiment, the intrabody comprises a single-chain Fv (“sFv”). sFvs are antibody fragments comprising the V_{H} and V_{L} domains of antibody, wherein these domains are present in a single polypeptide chain. Generally, the sFv polypeptide further comprises a polypeptide linker between the V_{H} and V_{L} domains which enables the sFv to form the desired structure for antigen binding. For a review of sFvs see Pluckthun in The Pharmacology of Monoclonal Antibodies, vol. 113, Rosenburg and Moore eds. Springer-Verlag, New York, pp. 269-315 (1994). In a further embodiment, the intrabody preferably does not encode an operable secretory sequence and thus remains within the cell (see generally Marasco, Wash., 1998, “Intrabodies: Basic Research and Clinical Gene Therapy Applications” Springer: New York).

Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250, which references are incorporated herein by reference in their entireties. Recombinant molecular biological techniques may also be used in the generation of intrabodies.

In one embodiment, intrabodies of the invention retain at least about 75% of the binding effectiveness of the complete antibody (i.e., having the entire constant domain as well as the variable regions) to the antigen. More preferably, the intrabody retains at least 85% of the binding effectiveness of the complete antibody. Still more preferably, the intrabody retains at least 90% of the binding effectiveness of the complete antibody. Even more preferably, the intrabody retains at least 95% of the binding effectiveness of the complete antibody.

In producing intrabodies, polynucleotides encoding variable region for both the V_{H} and V_{L} chains of interest can be cloned by using, for example, hybridoma mRNA or splenic mRNA as a template for PCR amplification of such domains (Huse et al., 1989, Science 246:1276). In one preferred embodiment, the polynucleotides encoding the V_{H} and V_{L} domains are joined by a polynucleotide sequence encoding a linker to make a single chain antibody (sFv). The sFv typically comprises a single peptide with the sequence V_{H}-linker-V_{L} or V_{L}-linker-V_{H}. The linker is chosen to permit the heavy chain and light chain to bind together in their proper conformational orientation (see for example, Huston, et al., 1991, Methods in Enzym. 203:46-121, which is incorporated herein by reference). In a further embodiment, the linker can span the distance between its points of fusion to each of the variable domains (e.g., 3.5 nm) to minimize distortion of the native Fv conformation. In such an embodiment, the linker is a polypeptide of at least 5 amino acid residues, at least 10 amino acid residues, at least 15 amino acid residues, or greater. In a further embodiment, the linker should not cause a steric interference with the V_{H} and V_{L} domains of the combining site. In such an embodiment, the linker is 35 amino acids or less, 30 amino acids or less, or 25 amino acids or less. Thus, in a most preferred embodiment, the linker is between 15-25 amino acid residues in length. In a further embodiment, the linker is hydrophilic and sufficiently flexible such that the V_{H} and V_{L} domains can adopt the conformation necessary to detect antigen. Intrabodies can be generated with different linker sequences inserted between identical V_{H} and V_{L} domains. A linker with the appropriate properties for a particular pair of V_{H} and V_{L} domains can be determined empirically by assessing the degree of antigen binding for each. Examples of linkers include, but are not limited to, those sequences disclosed in Table 1.

TABLE 1

| | |
|---|---|
| Sequence | SEQ ID NO. |

| | |
|---|---|
| (Gly Gly Gly Gly Ser)₃ | SEQ ID NO: 1 |
| Glu Ser Gly Arg Ser Gly Gly Gly Gly | SEQ ID NO: 2 |
| Ser Gly Gly Gly Gly Ser | |
| Glu Gly Lys Ser Ser Gly Ser Gly Ser | SEQ ID NO: 3 |
| Glu Ser Lys Ser Thr | |
| Glu Gly Lys Ser Ser Gly Ser Gly Ser | SEQ ID NO: 4 |
| Glu Ser Lys Ser Thr Gln | |
| Glu Gly Lys Ser Ser Gly Ser Gly Ser | SEQ ID NO: 5 |
| Glu Ser Lys Val Asp | |
| Gly Ser Thr Ser Gly Ser Gly Lys Ser | SEQ ID NO: 6 |
| Ser Glu Gly Lys Gly | |
| Lys Glu Ser Gly Ser Val Ser Ser Glu | SEQ ID NO: 7 |
| Gln Leu Ala Gln Phe Arg Ser Leu Asp | |
| Glu Ser Gly Ser Val Ser Ser Glu Glu | SEQ ID NO: 8 |
| Leu Ala Phe Arg Ser Leu Asp | |

In one embodiment, intrabodies are expressed in the cytoplasm. In other embodiments, the intrabodies are localized to various intracellular locations. In such embodiments, specific localization sequences can be attached to the intrabody polypeptide to direct the intrabody to a specific location. Intrabodies can be localized, for example, to the following intracellular locations: endoplasmic reticulum (Munro et al., 1987, Cell 48:899-907; Hangejorden et al., 1991, J. Biol. Chem. 266:6015); nucleus (Lanford et al., 1986, Cell 46:575; Stanton et al., 1986, PNAS 83:1772; Harlow et al., 1985, Mol. Cell Biol. 5:1605; Pap et al., 2002, Exp. Cell Res. 265:288-93); nucleolar region (Seomi et al., 1990, J Virology 64:1803; Kubota et al., 1989, Biochem. Biophys. Res. Comm. 162:963; Siomi et al., 1998, Cell 55:197); endosomal compartment (Bakke et al., 1990, Cell 63:707-716); mitochondrial matrix (Pugsley, A. P., 1989, “Protein Targeting”, Academic Press, Inc.); Golgi apparatus (Tang et al., 1992, J. Bio. Chem. 267:10122-6); liposomes (Letoumeur et al., 1992, Cell 69:1183); peroxisome (Pap et al., 2002, Exp. Cell Res. 265:288-93); trans Golgi network (Pap et al., 2002, Exp. Cell Res. 265:288-93); and plasma membrane (Marchildon et al., 1984, PNAS 81:7679-82; Henderson et al., 1987, PNAS 89:339-43; Rhee et al., 1987, J. Virol. 61:1045-53; Schultz et al., 1984, J. Virol. 133:431-7; Ootsuyama et al., 1985, Jpn. J Can. Res. 76:1132-5; Ratner et al., 1985, Nature 313:277-84). Examples of localization signals include, but are not limited to, those sequences disclosed in Table 2.

TABLE 2

| | | |
|---|---|---|
| Localization | Sequence | SEQ ID NO. |

| | | |
|---|---|---|
| endoplasmic | Lys Asp Glu Leu | SEQ ID NO: 9 |
| reticulum | | |
| endoplasmic | Asp Asp Glu Leu | SEQ ID NO: 10 |
| reticulum | | |
| endoplasmic | Asp Glu Glu Leu | SEQ ID NO: 11 |
| reticulum | | |
| endoplasmic | Gln Glu Asp Leu | SEQ ID NO: 12 |
| reticulum | | |
| endoplasmic | Arg Asp Glu Leu | SEQ ID NO: 13 |
| reticulum | | |
| nucleus | Pro Lys Lys Lys Arg Lys | SEQ ID NO: 14 |
| | Val | |
| nucleus | Pro Gln Lys Lys Ile Lys | SEQ ID NO: 15 |
| | Ser | |
| nucleus | Gln Pro Lys Lys Pro | SEQ ID NO: 16 |
| nucleus | Arg Lys Lys Arg | SEQ ID NO: 17 |
| nucleus | Lys Lys Lys Arg Lys | SEQ ID NO: 18 |
| nucleolar | Arg Lys Lys Arg Arg Gln | SEQ ID NO: 19 |
| region | Arg Arg Arg Ala His Gln | |
| nucleolar | Arg Gln Ala Arg Arg Asn | SEQ ID NO: 20 |
| region | Arg Arg Arg Arg Trp Arg | |
| | Glu Arg Gln Arg | |
| nucleolar | Met Pro Leu Thr Arg Arg | SEQ ID NO: 21 |
| region | Arg Pro Ala Ala Ser Gln | |
| | Ala Leu Ala Pro Pro Thr | |
| | Pro | |
| endosomal | Met Asp Asp Gln Arg Asp | SEQ ID NO: 22 |
| compartment | Leu Ile Ser Asn Asn Glu | |
| | Gln Leu Pro | |
| mitochondrial | Met Leu Phe Asn Leu Arg | SEQ ID NO: 23 |
| matrix | Xaa Xaa Leu Asn Asn Ala | |
| | Ala Phe Arg His Gly His | |
| | Asn Phe Met Val Arg Asn | |
| | Phe Arg Cys Gly Gln Pro | |
| | Leu Xaa | |
| peroxisome | Ala Lys Leu | SEQ ID NO: 24 |
| trans Golgi | Ser Asp Tyr Gln Arg Leu | SEQ ID NO: 25 |
| network | | |
| plasma | Gly Cys Val Cys Ser Ser Asn Pro | SEQ ID NO: 26 |
| membrane | | |
| plasma | Gly Gln Thr Val Thr Thr Pro Leu | SEQ ID NO: 27 |
| membrane | | |
| plasma | Gly Gln Glu Leu Ser Gln His Glu | SEQ ID NO: 28 |
| membrane | | |
| plasma | Gly Asn Ser Pro Ser Tyr Asn Pro | SEQ ID NO: 29 |
| membrane | | |
| plasma | Gly Val Ser Gly Ser Lys Gly Gln | SEQ ID NO: 30 |
| membrane | | |
| plasma | Gly Gln Thr Ile Thr Thr Pro Leu | SEQ ID NO: 31 |
| membrane | | |
| plasma | Gly Gln Thr Leu Thr Thr Pro Leu | SEQ ID NO: 32 |
| membrane | | |
| plasma | Gly Gln Ile Phe Ser Arg Ser Ala | SEQ ID NO: 33 |
| membrane | | |
| plasma | Gly Gln Ile His Gly Leu Ser Pro | SEQ ID NO: 34 |
| membrane | | |
| plasma | Gly Ala Arg Ala Ser Val Leu Ser | SEQ ID NO: 35 |
| membrane | | |
| plasma | Gly Cys Thr Leu Ser Ala Glu Glu | SEQ ID NO: 36 |
| membrane | | |

V_{H} and V_{L} domains are made up of the immunoglobulin domains that generally have a conserved structural disulfide bond. In embodiments where the intrabodies are expressed in a reducing environment (e.g., the cytoplasm), such a structural feature cannot exist. Mutations can be made to the intrabody polypeptide sequence to compensate for the decreased stability of the immunoglobulin structure resulting from the absence of disulfide bond formation. In one embodiment, the V_{H} and/or V_{L} domains of the intrabodies contain one or more point mutations such that their expression is stabilized in reducing environments (see Steipe et al., 1994, J. Mol. Biol. 240:188-92; Wirtz and Steipe, 1999, Protein Science 8:2245-50; Ohage and Steipe, 1999, J. Mol. Biol. 291:1119-28; Ohage et al., 1999, J. Mol. Biol. 291:1129-34).

Intrabody Proteins as Therapeutics

In one embodiment, the recombinantly expressed intrabody protein is administered to a patient. Such an intrabody polypeptide must be intracellular to mediate a prophylactic or therapeutic effect. In this embodiment of the invention, the intrabody polypeptide is associated with a “membrane permeable sequence”. Membrane permeable sequences are polypeptides capable of penetrating through the cell membrane from outside of the cell to the interior of the cell. When linked to another polypeptide, membrane permeable sequences can also direct the translocation of that polypeptide across the cell membrane as well.

In one embodiment, the membrane permeable sequence is the hydrophobic region of a signal peptide (see, e.g., Hawiger, 1999, Curr. Opin. Chem. Biol. 3:89-94; Hawiger, 1997, Curr. Opin. Immunol. 9:189-94; U.S. Pat. Nos. 5,807,746 and 6,043,339, which are incorporated herein by reference in their entireties). The sequence of a membrane permeable sequence can be based on the hydrophobic region of any signal peptide. The signal peptides can be selected, e.g., from the SIGPEP database (see e.g., von Heijne, 1987, Prot. Seq. Data Anal. 1:41-2; von Heijne and Abrahmsen, 1989, FEBS Lett. 224:439-46). When a specific cell type is to be targeted for insertion of an intrabody polypeptide, the membrane permeable sequence is preferably based on a signal peptide endogenous to that cell type. In another embodiment, the membrane permeable sequence is a viral protein (e.g., Herpes Virus Protein VP22) or fragment thereof (see e.g., Phelan et al., 1998, Nat. Biotechnol. 16:440-3). A membrane permeable sequence with the appropriate properties for a particular intrabody and/or a particular target cell type can be determined empirically by assessing the ability of each membrane permeable sequence to direct the translocation of the intrabody across the cell membrane. Examples of membrane permeable sequences include, but are not limited to, those sequences disclosed in Table 3.

TABLE 3

| | |
|---|---|
| Sequence | SEQ ID NO. |

| | |
|---|---|
| Ala Ala Val Ala Leu Leu Pro Ala Val | SEQ ID NO: 37 |
| Leu Leu Ala Leu Leu Ala Pro | |
| Ala Ala Val Leu Leu Pro Val Leu Leu | SEQ ID NO: 38 |
| Ala Ala Pro | |
| Val Thr Val Leu Ala Leu Gly Ala Leu | SEQ ID NO: 39 |
| Ala Gly Val Gly Val Gly | |

In another embodiment, the membrane permeable sequence can be a derivative. In this embodiment, the amino acid sequence of a membrane permeable sequence has been altered by the introduction of amino acid residue substitutions, deletions, additions, and/or modifications. For example, but not by way of limitation, a polypeptide may be modified, e.g., by glycosylation, acetylation, pegylation, phosphorylation, amidation, derivatization by known protecting/blocking groups, proteolytic cleavage, linkage to a cellular ligand or other protein, etc. A derivative of a membrane permeable sequence polypeptide may be modified by chemical modifications using techniques known to those of skill in the art, including, but not limited to specific chemical cleavage, acetylation, formylation, metabolic synthesis of tunicamycin, etc. Further, a derivative of a membrane permeable sequence polypeptide may contain one or more non-classical amino acids. In one embodiment, a polypeptide derivative possesses a similar or identical function as an unaltered polypeptide. In another embodiment, a derivative of a membrane permeable sequence polypeptide has an altered activity when compared to an unaltered polypeptide. For example, a derivative membrane permeable sequence polypeptide can translocate through the cell membrane more efficiently or be more resistant to proteolysis.

The membrane permeable sequence can be attached to the intrabody in a number of ways. In one embodiment, the membrane permeable sequence and the intrabody are expressed as a fusion protein. In this embodiment, the nucleic acid encoding the membrane permeable sequence is attached to the nucleic acid encoding the intrabody using standard recombinant DNA techniques (see e.g., Rojas et al., 1998, Nat. Biotechnol. 16:370-5). In a further embodiment, there is a nucleic acid sequence encoding a spacer peptide placed in between the nucleic acids encoding the membrane permeable sequence and the intrabody. In another embodiment, the membrane permeable sequence polypeptide is attached to the intrabody polypeptide after each is separately expressed recombinantly (see e.g., Zhang et al., 1998, PNAS 95:9184-9). In this embodiment, the polypeptides can be linked by a peptide bond or a non-peptide bond (e.g. with a crosslinking reagent such as glutaraldehyde or a thiazolidino linkage see e.g., Hawiger, 1999, Curr. Opin. Chem. Biol. 3:89-94) by methods standard in the art.

The administration of the membrane permeable sequence-intrabody polypeptide can be by parenteral administration, e.g., by intravenous injection including regional perfusion through a blood vessel supplying the tissues(s) or organ(s) having the target cell(s), or by inhalation of an aerosol, subcutaneous or intramuscular injection, topical administration such as to skin wounds and lesions, direct transfection into, e.g., bone marrow cells prepared for transplantation and subsequent transplantation into the subject, and direct transfection into an organ that is subsequently transplanted into the subject. Further administration methods include oral administration, particularly when the complex is encapsulated, or rectal administration, particularly when the complex is in suppository form. A pharmaceutically acceptable carrier includes any material that is not biologically or otherwise undesirable, i.e., the material may be administered to an individual along with the selected complex without causing any undesirable biological effects or interacting in a deleterious manner with any of the other components of the pharmaceutical composition in which it is contained.

Conditions for the administration of the membrane permeable sequence-intrabody polypeptide can be readily be determined, given the teachings in the art (see e.g., Remington 's Pharmaceutical Sciences, 18^{th} Ed., E. W. Martin (ed.), Mack Publishing Co., Easton, Pa. (1990)). If a particular cell type in vivo is to be targeted, for example, by regional perfusion of an organ or section of artery/blood vessel, cells from the target tissue can be biopsied and optimal dosages for import of the complex into that tissue can be determined in vitro to optimize the in vivo dosage, including concentration and time length. Alternatively, culture cells of the same cell type can also be used to optimize the dosage for the target cells in vivo.

Intrabody Gene Therapy as Therapeutic

In another embodiment, a polynucleotide encoding an intrabody is administered to a patient (e.g., as in gene therapy). In this embodiment, methods as described in Section 5.7.1 can be used to administer the polynucleotide of the invention.

5.2.1.2 Methods of Producing Antibodies

The EphA2 agonistic antibodies or fragments thereof can be produced by any method known in the art for the synthesis of antibodies, in particular, by chemical synthesis or preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with EphA2 (either the full length protein or a domain thereof, e.g., the extracellular domain or the cytoplasmic tail domain) and once an immune response is detected, e.g., antibodies specific for EphA2 are detected in the mouse serum, the mouse spleen is harvested and splenocytes isolated. The splenocytes are then fused by well known techniques to any suitable myeloma cells, for example cells from cell line SP20 (available from the ATCC) or NHO cells. Hybridomas are selected and cloned by limited dilution. Hybridoma clones are assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody of the invention wherein, preferably, the hybridoma is generated by fusing splenocytes isolated from a mouse immunized with EphA2 or fragment thereof with myeloma cells and then screening the hybridomas resulting from the fusion for hybridoma clones that secrete an antibody able to bind and agonize EphA2.

Antibody fragments which recognize specific EphA2 epitopes may be generated by any technique known to those of skill in the art. For example, Fab and F(ab′)₂ fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding V_{H} and V_{L} domains are amplified from animal cDNA libraries (e.g., human or murine cDNA libraries of lymphoid tissues). The DNA encoding the V_{H} and V_{L} domains are recombined together with an sFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the V_{H} and V_{L} domains are usually recombinantly fused to either the phage gene III or gene VIII. Phage expressing an antigen binding domain that binds to the EphA2 epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177; Kettleborough et al., 1994, Eur. J. Immunol. 24:952-958; Persic et al., 1997, Gene 187:9; Burton et al., 1994, Advances in Immunology 57:191-280; International Application No. PCT/GB91/01134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

Phage may be screened for EphA2 binding, particularly to the extracellular domain of EphA2, and agonizing activity such as, e.g., increasing EphA2 cytoplasmic tail phosphorylation, increasing EphA2 autophosphorylation, reducing EphA2 activity (other than autophosphorylation), decreasing a pathology-causing cell phenotype (e.g., secretion of mucin, differentiation of EphA2-expressing cells into a mucin-secreting cell, secretion of inflammatory factors, cell hyperproliferation, cell migration, cell volume and/or secretion of extracellular matrix molecules or matrix metalloproteinases). (see e.g., Section 5.5 for methods of screening.)

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Patent Publication No. WO 92/22324; Mullinax et al., 1992, BioTechniques 12:864; Sawai et al., 1995, AJRI 34:26; and Better et al., 1988, Science 240:1041 (said references incorporated by reference in their entireties).

To generate whole antibodies, PCR primers including V_{H} or V_{L} nucleotide sequences, a restriction site, and a flanking sequence to protect the restriction site can be used to amplify the V_{H} or V_{L} sequences in sFv clones. Utilizing cloning techniques known to those of skill in the art, the PCR amplified V_{H} domains can be cloned into vectors expressing a V_{H} constant region, e.g., the human gamma 4 constant region, and the PCR amplified V_{L} domains can be cloned into vectors expressing a V_{L} constant region, e.g., human kappa or lambda constant regions. Preferably, the vectors for expressing the V_{H} or V_{L} domains comprise an EF-1α promoter, a secretion signal, a cloning site for the variable domain, constant domains, and a selection marker such as neomycin. The V_{H} and V_{L} domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human, humanized or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Patent Publication Nos. WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Patent Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Medarex (Princeton, N.J.) can be engaged to provide human antibodies directed against a selected antigen using technology similar to that described above.

A chimeric antibody is a molecule in which different portions of the antibody are derived from different immunoglobulin molecules such as antibodies having a variable region derived from a non-human antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,816,397, which are incorporated herein by reference in their entirety. Chimeric antibodies comprising one or more CDRs from a non-human species and framework regions from a human immunoglobulin molecule can be produced using a variety of techniques known in the art including, for example, CDR-grafting (EP 239,400; International Patent Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, PNAS 91:969), and chain shuffling (U.S. Pat. No. 5,565,332). In one embodiment, a chimeric antibody of the invention immunospecifically binds EphA2 and comprises one, two, or three V_{L} CDRs having an amino acid sequence of any of the V_{L} CDRs of Eph099B-102.147, Eph099B-208.261, Eph099B-210.248, B233, EA2, or EA5 within human framework regions. In another embodiment, a chimeric antibody of the invention immunospecifically binds EphA2 and comprises one, two, or three V_{H} CDRs having an amino acid sequence of any of the V_{H} CDRs of Eph099B-102.147, Eph099B-208.261, Eph099B-210.248, B233, EA2, or EA5 within human framework regions. In another embodiment, a chimeric antibody of the invention immunospecifically binds EphA2 and comprises one, two, or three V_{L} CDRs having an amino acid sequence of any of the V_{L} CDRs of Eph099B-102.147, Eph099B-208.261, Eph099B-210.248, B233, EA2, or EA5 and further comprises one, two, or three V_{H} CDRs having an amino acid sequence of any of the V_{H} CDRs of Eph099B-102.147, Eph099B-208.261, Eph099B-210.248, B233, EA2, or EA5 within human framework regions. In a preferred embodiment, a chimeric antibody of the invention immunospecifically binds EphA2 and comprises three V_{L} CDRs having an amino acid sequence of any of the V_{L} CDRs of Eph099B-102.147, Eph099B-208.261, Eph099B-210.248, B233, EA2, or EA5 and three V_{H} CDRs having an amino acid sequence of any of the V_{H} CDRs of Eph099B-102.147, Eph099B-208.261, Eph099B-210.248, B233, EA2, or EA5 within human framework regions.

Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann et al., 1988, Nature 332:323, which are incorporated herein by reference in their entireties.)

5.2.2 EphA2 Ligands as Polypeptide Agonistic Agents

In another embodiment, a polypeptide agonistic agent is an EphA2 ligand (e.g., Ephrin A1) or fragment thereof that is capable of binding EphA2 and agonizing EphA2 (e.g., increases EphA2 cytoplasmic tail phosphorylation, increases EphA2 degradation, decreases survival of EphA2 expressing cells, increases EphA2 autophosphorylation, reduces EphA2 activity (other than autophosphorylation), and/or decreases a pathology-causing cell phenotype). In a specific embodiment, a fragment of EphA2 ligand which retains its ability to bind and agonize EphA2 (e.g., the Ephrin A1 extracellular domain) is used in the methods of the invention. In another specific embodiment, a fusion protein comprises the fragment of EphA2 ligand which retains its ability to bind and agonize EphA2 (e.g., the extracellular domain of Ephrin A1 fused to immunoglobulin heavy chain, see Pratt and Kinch, 2002, Oncogene 21:7690-9, which is incorporated herein by reference in its entirety). In a preferred embodiment, the EphA2 ligand fragment is soluble. Fragments of EphA2 ligand can be made (e.g., using EphA2 ligand sequences known in the art such as the Ephrin A1 sequence of Genbank Accession No. BC032698) and assayed for the ability to bind and agonize EphA2. In one embodiment, the fragment comprises amino acid residues 1 to approximately 400, 500, or 600 of EphA2. In a more specific embodiment, the fragment is amino acid residues 1-534 of EphA2. Any method known in the art to detect binging between proteins may be used including, but not limited to, affinity chromatography, size exclusion chromatography, electrophoretic mobility shift assay. Polypeptide agonistic agents of the invention that are EphA2 ligand fragments include polypeptides that are 100%, 98%, 95%, 90%, 85%, 80%, 75%, 70%, 65%, 60%, 55%, 50%, 45%, 40% identical to endogenous EphA2 ligand sequences. The determination of percent identity of two amino acid sequences can be determined by any method known to one skilled in the art, including BLAST protein searches.

5.2.3 Modified Polypeptide Agonistic Agents

The polypeptide agonistic agents used in the methods of the invention (e.g., antibodies or EphA2 polypeptide ligands or fragments thereof) include derivatives that are modified, i.e, by the covalent attachment of any type of molecule to the antibody such that covalent attachment does not substantially alter the immunospecificity of the antibody. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The methods of the present invention also encompass the use of antibodies or fragments thereof that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the polypeptide agonistic agents in mammals, preferably humans, result in higher serum concentration of said polypeptide agonistic agents in the mammals, and thus, reduces the frequency of the administration of said polypeptide agonistic agents and/or reduces the amount of said polypeptide agonistic agents to be administered. Polypeptide agonistic agents having increased in vivo half-lives can be generated by techniques known to those of skill in the art. For example, polypeptide agonistic agents with increased in vivo half-lives can be generated by modifying (e.g., substituting, deleting or adding) amino acid residues identified as involved in the interaction between the Fc domain and the FcRn receptor (see, e.g., International Patent Publication No. WO 97/34631 and U.S. patent application Ser. No. 10/020,354 filed Dec. 12, 2001 entitled “Molecules With Extended Half-Lives, Compositions and Uses Thereof,” which are incorporated herein by reference in their entireties). Polypeptide agonistic agents with increased in vivo half-lives can be generated by attaching to said polypeptide agonistic agents polymer molecules such as high molecular weight polyethyleneglycol (PEG). PEG can be attached to said polypeptide agonistic agents with or without a multifunctional linker either through site-specific conjugation of the PEG to the N- or C-terminus of said polypeptide agonistic agents or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the polypeptide agonistic agents. Unreacted PEG can be separated from polypeptide agonistic agent-PEG conjugates by, e.g., size exclusion or ion-exchange chromatography.

5.2.3.1 Polynucleotides Encoding Polypeptide Agonistic Agents

The EphA2 polypeptide agonistic agents of the invention include polypeptides produced from polynucleotides that hybridize to polynucleotides which encode polypeptides disclosed in Sections 5.2.1 and 5.2.2 above. In one embodiment, antibodies of the invention include EphA2 monoclonal antibodies produced from polynucleotides that hybridize to polynucleotides encoding monoclonal antibodies that agonize EphA2 in one or more of the assays described in Section 5.5. In specific embodiments, the methods of the invention use EphA2 monoclonal antibodies produced from polynucleotides that hybridize to polynucleotides encoding monoclonal antibodies Eph099B-102.147, Eph099B-208.261, or Eph099B-210.248 deposited with the ATCC on Aug. 7, 2002 and assigned accession numbers PTA-4572, PTA-4573, and PTA-4574, respectively or polynucleotides encoding monoclonal antibody B233). In another embodiment, EphA2 ligand polypeptides used in the methods of the invention include polypeptides produced from polynucleotides that hybridize to polynucleotides encoding a EphA2 binding domain of an EphA2 ligand (e.g., Ephrin A1).

Conditions for hybridization can be high stringency, intermediate stringency, or lower stringency. For example, conditions for stringent hybridization include, but are not limited to, hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The polynucleotides encoding polypeptide agonistic agents of the invention may be obtained, and the nucleotide sequence of the polynucleotides determined, by any method known in the art. Such a polynucleotide encoding a polypeptide agonistic agent used in the methods of the invention may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the polypeptide, annealing and ligating of those oligonucleotides, and then amplification of the ligated oligonucleotides by PCR.

Alternatively, a polynucleotide encoding a polypeptide agonistic agent used in the methods of the invention may be generated from nucleic acid from a suitable source. If a clone containing a nucleic acid encoding a particular polypeptide is not available, but the sequence of the polypeptide is known, a nucleic acid encoding the polypeptide may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention or cells expressing a Epha2 ligand) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or EphA2 ligand. Amplified nucleic acids generated by PCR may then be cloned into replicable cloning vectors using any method well known in the art.

Once the nucleotide sequence of the polypeptide agonistic agent used in the methods of the invention is determined, the nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties) to generate polypeptides having a different amino acid sequence, for example to create amino acid substitutions, deletions, and/or insertions.

Standard techniques known to those skilled in the art can be used to introduce mutations in the nucleotide sequence encoding a polypeptide agonistic agent, or fragment thereof, including, e.g., site-directed mutagenesis and PCR-mediated mutagenesis, which results in amino acid substitutions. Preferably, the derivatives include less than 15 amino acid substitutions, less than 10 amino acid substitutions, less than 5 amino acid substitutions, less than 4 amino acid substitutions, less than 3 amino acid substitutions, or less than 2 amino acid substitutions relative to the original polypeptide agonistic agent or fragment thereof. In a preferred embodiment, the derivatives have conservative amino acid substitutions made at one or more predicted non-essential amino acid residues.

The present invention also encompasses the use of antibodies or antibody fragments comprising the amino acid sequence of any EphA2 agonistic antibodies of the invention with mutations (e.g., one or more amino acid substitutions) in the framework or variable regions. Preferably, mutations in these antibodies maintain or enhance the avidity and/or affinity of the antibodies for the particular antigen to which they immunospecifically bind. Standard techniques known to those skilled in the art (e.g., immunoassays or ELISA assays) can be used to assay the degree of binding between a polypeptide agonistic agent and its binding partner. In a specific embodiment, when a polypeptide agonistic agent is an antibody, binding to an EphA2 antigen can be assessed. In another embodiment, when a polypeptide agonistic agent is an EphA2 ligand, binding to EphA2 can be assessed.

5.2.3.2 Recombinant Production of Polypeptide Agonistic Agents

Recombinant expression of a polypeptide agonistic agent (including, but not limited to derivatives, analogs or fragments thereof) requires construction of an expression vector containing a polynucleotide that encodes the polypeptide. Once a polynucleotide encoding a polypeptide agonistic agent has been obtained, a vector for the production of the polypeptide agonistic agent may be produced by recombinant DNA technology using techniques well known in the art. Methods which are well known to those skilled in the art can be used to construct expression vectors containing polypeptide coding sequences and appropriate transcriptional and translational control signals. Thus, methods for preparing a protein by expressing a polynucleotide containing are described herein. These methods include, for example, in vitro recombinant DNA techniques, synthetic techniques, and in vivo genetic recombination. The invention, thus, provides replicable vectors comprising a nucleotide sequence encoding a EphA2 agonistic polypeptide agent.

The expression vector is transferred to a host cell by conventional techniques and the transfected cells are then cultured by conventional techniques to produce a polypeptide agonistic agent. Thus, the invention includes host cells containing a polynucleotide encoding a polypeptide agonistic agent or fragments thereof operably linked to a heterologous promoter.

A variety of host-expression vector systems may be utilized to express polypeptide agonistic agents (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express a polypeptide agonistic agent of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing polypeptide agonistic agent coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant polypeptide agonistic agent, are used for the expression of a polypeptide agonistic agent. For example, mammalian cells such as Chinese hamster ovary cells (CHO), in conjunction with a vector such as the major intermediate early gene promoter element from human cytomegalovirus is an effective expression system for polypeptide agonistic agents, especially antibody polypeptide agonistic agents (Foecking et al., 1986, Gene 45:101; and Cockett et al., 1990, BioTechnology 8:2). In a specific embodiment, the expression of nucleotide sequences encoding a polypeptide agonistic agent is regulated by a constitutive promoter, inducible promoter or tissue specific promoter.

In bacterial systems, a number of expression vectors may be advantageously selected depending upon the use intended for the polypeptide being expressed. For example, when a large quantity of such a protein is to be produced, for the generation of pharmaceutical compositions, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the polypeptide coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the polypeptide agonistic agent in infected hosts (e.g., see Logan & Shenk, 1984, PNAS 81:355-359). Specific initiation signals may also be required for efficient translation of inserted polypeptide coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, WI 38, BT483, Hs578T, HTB2, BT20 and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the polypeptide agonistic agent. Such engineered cell lines may be particularly useful in screening and evaluation of compositions that interact directly or indirectly with the polypeptide agonistic agent.

A number of selection systems may be used, including but not limited to, the herpes simplex virus thymidine kinase (Wigler et al., 1977, Cell 11:223), glutamine synthetase, hypoxanthine guanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, gs-, hgprt- or aprt-cells, respectively. Also, antimetabolite resistance can be used as the basis of selection for the following genes: dhfr, which confers resistance to methotrexate (Wigler et al., 1980, PNAS 77:357; O'Hare et al., 1981, PNAS 78:1527); gpt, which confers resistance to mycophenolic acid (Mulligan & Berg, 1981, PNAS 78:2072); neo, which confers resistance to the aminoglycoside G-418 (Wu and Wu, 1991, Biotherapy 3:87; Tolstoshev, 1993, Ann. Rev. Pharmacol. Toxicol. 32:573; Mulligan, 1993, Science 260:926; and Morgan and Anderson, 1993, Ann. Rev. Biochem. 62: 191; May, 1993, TIB TECH 11:155-); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of a polypeptide agonistic agent can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing polypeptide agonistic agent is amplifiable, increase in the level of inhibitor present in culture of host cell will increase the number of copies of the marker gene. Since the amplified region is associated with the polypeptide agonistic agent gene, production of the polypeptide agonistic agent will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, PNAS 77:2197). The coding sequences for the heavy and light chains may comprise cDNA or genomic DNA.

Once a polypeptide agonistic agent of the invention has been produced by recombinant expression, it may be purified by any method known in the art for purification of a polypeptide, for example, by chromatography (e.g., ion exchange, affinity, and sizing column chromatography), centrifugation, differential solubility, or by any other standard technique for the purification of proteins. Further, the polypeptide agonistic agents may be fused to heterologous polypeptide sequences described herein or otherwise known in the art to facilitate purification.

Polypeptide agonistic agents of the invention that are antibodies may be expressed using vectors which already include the nucleotide sequence encoding the constant region of the antibody molecule (see, e.g., U.S. Pat. Nos. 5,919,900; 5,747,296; 5,789,178; 5,591,639; 5,658,759; 5,849,522; 5,122,464; 5,770,359; 5,827,739; International Patent Publication Nos. WO 89/01036; WO 89/10404; Bebbington et al., 1992, BioTechnology 10:169). The variable domain of the antibody may be cloned into such a vector for expression of the entire heavy, the entire light chain, or both the entire heavy and light chains. In preferred embodiments for the expression of double-chained antibodies, vectors encoding both the heavy and light chains may be co-expressed in the host cell for expression of the entire immunoglobulin molecule.

5.3 Polynucleotide Agonistic Agents

In addition EphA2 polypeptide agonistic agents of the invention, nucleic acid molecules can be used in methods of the invention. Nucleic acid molecules including, but not limited to, antisense, ribozyme, and RNA interference technology can be used to decrease EphA2 expression. Nucleotide agonistic agents can be administered to a patient according to methods described in Section 5.7.1.

5.3.1 Antisense

The present invention encompasses antisense nucleic acid molecules, i.e., molecules which are complementary to all or part of a sense nucleic acid encoding EphA2, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids. In one embodiment, the antisense nucleic acid molecule is

| | |
|---|---|
| 5′-CCAGCAGTACCGCTTCCTTGCCCTGCGGCC | (SEQ ID NO: 44) |
| G-3′. | |

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, i.e., EphA2).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids Res. 15:6625). The antisense nucleic acid molecule can also comprise a 2′-o-methylribonucleotide (Inoue et al., 1987, Nucleic Acids Res. 15:6131) or a chimeric RNA-DNA analogue (Inoue et al., 1987, FEBS Lett. 215:327).

5.3.2. Ribozymes

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes; described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding EphA2 can be designed based upon the nucleotide sequence of EphA2. For example, a derivative of a Tetrahymena L-19 IVS RNA can be constructed in which the nucleotide sequence of the active site is complementary to the nucleotide sequence to be cleaved in U.S. Pat. Nos. 4,987,071 and 5,116,742. Alternatively, an mRNA encoding a polypeptide of the invention can be used to select a catalytic RNA having a specific ribonuclease activity from a pool of RNA molecules. See, e.g., Bartel and Szostak, 1993, Science 261:1411.

5.3.3 RNA Interference

In certain embodiments, an RNA interference (RNAi) molecule is used to decrease EphA2 expression. RNA interference (RNAi) is defined as the ability of double-stranded RNA (dsRNA) to suppress the expression of a gene corresponding to its own sequence. RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Double-stranded (ds) RNA can be used to interfere with gene expression in mammals (Wianny & Zernicka-Goetz, 2000, Nature Cell Biology 2: 70-75; incorporated herein by reference in its entirety). dsRNA is used as inhibitory RNA or RNAi of the function of EphA2 to produce a phenotype that is the same as that of a null mutant of EphA2 (Wianny & Zemicka-Goetz, 2000, Nature Cell Biology 2: 70-75).

5.4 Prophylactic/Therapeutic Methods

The present invention encompasses methods for treating, preventing, or managing a disorder associated with overexpression of EphA2 and/or non-neoplastic cellular hyperproliferation, particularly of epithelial cells (e.g., as in asthma, COPD, lung fibrosis, asbestosis, IPF, DIP, UIP, kidney fibrosis, liver fibrosis, other fibroses, bronchial hyper responsiveness, psoriasis, seborrheic dermatitis, and cystic fibrosis) and endothelial cells (e.g., as in restenosis, hyperproliferative vascular disease, Behcet's Syndrome, atherosclerosis, and macular degeneration), in a subject comprising administering one or more EphA2 agonistic agents of the invention. In one embodiment, the agents of the invention can be administered in combination with one or more other therapeutic agents useful in the treatment, prevention or management of disorders associated with overexpression of EphA2 and/or non-neoplastic cell hyperproliferative disorders. In certain embodiments, one or more EphA2 agonistic agents of the invention are administered to a mammal, preferably a human, in combination with one or more other therapeutic agents useful for the treatment of a non-neoplastic hyperproliferative cell or excessive cell accumulation disorder.

In preferred embodiments, the one or more EphA2 agonistic agents of the invention is an antibody, preferably a monoclonal antibody. In more preferred embodiments, the EphA2 agonistic antibodies of the invention are Eph099B-102.147, Eph099B-208.261, Eph099B-210.248, B233, EA2, or EA5. In certain preferred embodiments, antibodies of the invention have been humanized. In other embodiments, variants of Eph099B-102.147, Eph099B-208.261, Eph099B-210.248, or B233, e.g., with one or more amino acid substitutions, particularly in the variable domain, are provided that have increased activity, binding ability, etc., as compared to Eph099B-102.147, Eph099B-208.261, Eph099B-210.248, B233, EA2, or EA5.

In another specific embodiment, the therapeutic and prophylactic methods of the invention comprise administration of an inhibitor of EphA2 expression, such as but not limited to, antisense nucleic acids specific for EphA2, double stranded EphA2 RNA that mediates RNAi, anti-EphA2 ribozymes, etc. (see Section 5.3 infra).

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity of the non-neoplastic hyperproliferative disorder, the route of administration, as well as age, body weight, response, and the past medical history of the patient. Suitable regimens can be selected by one skilled in the art by considering such factors and by following, for example, dosages reported in the literature and recommended in the Physician 's Desk Reference (56^{th} ed., 2002).

5.4.1 Patient Population

The invention provides methods for treating, preventing, and managing a non-neoplastic disorder associated with EphA2 overexpression, cellular hyperproliferation, particularly of epithelial and endothelial cells, or increased mucin production by administrating to a subject in need thereof a therapeutically or prophylactically effective amount of one or more EphA2 agonistic agents of the invention. The subject is preferably a mammal such as a non-primate (e.g., cows, pigs, horses, cats, dogs, rats, etc.) and a primate (e.g., monkey, such as a cynomolgous monkey and a human). In a preferred embodiment, the subject is a human.

The methods and compositions of the invention comprise the administration of one or more EphA2 agonistic agents of the invention to patients suffering from a non-neoplastic hyperproliferative disorder or expected to suffer from a non-neoplastic hyperproliferative cell or excessive cell accumulation disorder, e.g., have a genetic predisposition for a non-neoplastic hyperproliferative cell or excessive cell accumulation disorder (see e.g., U.S. Pat. No. 6,387,615 and International Patent Publication No. WO 95/05481) or previously have suffered from a non-neoplastic hyperproliferative cell or excessive cell accumulation disorder in the past or have been exposed to tobacco smoke or have been infected or previously infected with an upper respiratory tract infection (e.g., RSV, HMPV, or PIV) or have had angioplasty. Such patients may or may not have been previously treated for a non-neoplastic hyperproliferative cell or excessive cell accumulation disorder, e.g., with a non-EphA2-based therapeutic. The methods and compositions of the invention may be used as a first line or second line treatment. Included in the invention is also the treatment of patients currently undergoing non-EphA2-based therapies to treat a non-neoplastic hyperproliferative cell or excessive cell accumulation disorder or patients refractory to one or more non-EphA2-based therapies. The methods and compositions of the invention can be used before any adverse effects or intolerance of the non-EphA2 based therapies occurs. The invention also encompasses methods for administering one or more EphA2 agonistic agents of the invention to treat or ameliorate symptoms in refractory patients. The invention also encompasses methods for administering one or more EphA2 agonistic agents of the invention to prevent the onset or recurrence of a non-neoplastic hyperproliferative cell or excessive cell accumulation disorder in patients predisposed to having a non-neoplastic hyperproliferative cell or excessive cell accumulation disorder.

In one embodiment, a patient expected to suffer from a hyperproliferative epithelial cell disorder (e.g., asthma or COPD) is a patient who has or has had a respiratory viral infection. In a further embodiment, the respiratory viral infection is respiratory syncytial virus (RSV). In a specific embodiments, the patient who has or has had a respiratory viral infection is a human child, infant, or an infant born prematurely (see e.g., Zhoa et al., 2002, Pediatr. Allergy Immunol. 13:47-50; Message and Johnston, 2002, Br. Med. Bull. 61:29-43; Klinnert et al., 2001, Pediatrics 108:E69; Sigurs, 2002, Respiratory Res. 4:S8-S14).

In other embodiments, the invention also provides methods of treatment of non-neoplastic hyperproliferative cell or excessive cell accumulation disorders as alternatives to current therapies. In one embodiment, the current therapy has proven or may prove too toxic (i.e., results in unacceptable or unbearable side effects) for the patient. In another embodiment, the EphA2-based therapy has decreased side effects as compared to the current therapy. In another embodiment, the patient has proven refractory to the current therapy. In such embodiments, the invention provides administration of one or more EphA2 agonistic agents of the invention without any other non-neoplastic hyperproliferative cell or excessive cell accumulation disorder therapies. In certain embodiments, one or more EphA2 agonistic agents of the invention can be administered to a patient in need thereof instead of another therapy to treat non-neoplastic hyperproliferative cell or excessive cell accumulation disorders.

In one embodiment, the non-EphA2 based therapy is EphA4-based therapy.

In another embodiment, the hyperproliferative disorder is asthma and the non-EphA2 based therapy is, e.g., inhaled beta 2 agonists, inhaled corticosteroids, retinoic acid, anti-IgE antibodies, phosphodiesterase inhibitors, leukotriene antagonists, anti IL-9 antibody, and/or anti-mucin therapies (e.g., anti hCLCA1 therapy such as Lomucin™).

In another embodiment, the hyperproliferative disorder is COPD and the non-EphA2 based therapy is, e.g., tiotropium and/or ipratropium. In another embodiment, the hyperproliferative disorder is lung fibrosis and the non-EphA2 based therapy is, e.g., recombinant human relaxin such as ConXn™, methylprednisolone, cyclophosphamid, corticosteroids, azathioprine, cyclophosphamide, penicillamine, colchicine, cyclosporine and/or prednisolone.

In another embodiment, the hyperproliferative disorder is bronchial hyper responsiveness and the non-EphA2 based therapy is, e.g., budesonide, zafirlukast, beclomethasone dipropionate, budesonide, angiotensin II type 1 (AT1) receptor antagonist such as candesartan cilexetil and/or antisense oligonucleotide targeting the adenosine A(1) receptor such as EPI-2010™.

In another embodiment, the hyperproliferative disorder is psoriasis and the non-EphA2 based therapy is, e.g., corticosteroids, calcipotriene, coal tar, anthralin, retinoid, salicylic acid, moisturizers and/or phototherapy.

In another embodiment, the hyperproliferative disorder is seborrheic dermatitis and the non-EphA2 based therapy is, e.g., ciclopiroxolamine, ketoconazole, zinc pyrithione, terbinafine and/or pimecrolimus.

In another embodiment, the hyperproliferative disorder is restenosis and the non-EphA2 based therapy is, e.g., paclitaxel, doxorubicin, dipyridamole, clopidogrel and/or aspirin.

In another embodiment, the hyperproliferative disorder is hyperproliferative vascular disease and the non-EphA2 based therapy is, e.g., cyclin-dependent kinase inhibitors, bromocriptine and/or IL-2 receptor-specific chimeric toxin such as DAB486-IL-2™.

In another embodiment, the hyperproliferative disorder is Behcet's Syndrome and the non-EphA2 based therapy is, e.g., corticosteroids, prednisone, or immunosuppressive drugs such as azathioprine, chlorambucil, cyclosporine, colchicine and/or cyclophosphamide.

In another embodiment, the hyperproliferative disorder is atherosclerosis and the non-EphA2 based therapy is, e.g., beta blockers, fibrinolytic/thrombolytic therapy, raloxifene and/or statin therapy.

In another embodiment, the hyperproliferative disorder is macular degeneration and the non-EphA2 based therapy is, e.g., laser surgery and/or high levels of antioxidants and zinc.

In one embodiment, the EphA2 agonistic agent is an antibody. In a further embodiment, the EphA2 antibody is one or more of Eph099B-102.147, Eph099B-208.261, Eph099B-210.248, B233, EA2, or EA5.

In one embodiment, the non-neoplastic hyperproliferative disorder is not asthma. In another embodiment, the non-neoplastic hyperproliferative disorder is not COPD. In another embodiment, the non-neoplastic hyperproliferative disorder is not psoriasis. In another embodiment, the non-neoplastic hyperproliferative disorder is not restenosis.

5.4.2 Other Prophylactic/Therapeutic Agents

In some embodiments, the invention provides methods for treating a patient's non-neoplastic hyperproliferative cell or excessive cell accumulation disorder by administering one or more EphA2 agonistic agents of the invention in combination with any other therapy for a non-neoplastic hyperproliferative cell or excessive cell accumulation disorder (e.g., those therapies mentioned above) or that reduces the symptoms of a non-neoplastic hyperproliferative cell or excessive cell accumulation disorder. Administration of the therapeutic/prophylactic agents to a patient can be at exactly the same time or in a sequence within a time interval such that the agents can act together to provide an increased benefit than if they were administered otherwise. For example, each therapeutic/prophylactic agent may be administered in any order at different points of time; however, if not administered at the same time, they should administered sufficiently close in time so as to provide the desired therapeutic or prophylactic effect. Each therapeutic/prophylactic agent can be administered separately, in any appropriate form and by any suitable route.

In various embodiments, the prophylactic or therapeutic agents are administered less than 5 minutes apart, less than 30 minutes apart, 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

In one embodiment, EphA2 agonistic agents of the invention are administered in combination with a therapy currently known to treat a hyperproliferative cell or excessive cell accumulation disorder (see e.g., Section 5.4.1 supra). In another embodiment, EphA2 agonistic agents of the invention are administered in combination with an immunomodulatory agent, anti-viral agent that decreases the replication of a respiratory virus, bronchodilator, or anti-mucin therapy. In another embodiment, EphA2 agonistic agents of the invention are administered in combination with a therapy currently known to treat a non-neoplastic hyperproliferative cell or excessive cell accumulation disorder and an immunomodulatory agent, an anti-viral agent that decreases the replication of a respiratory virus, a bronchodilator, or an anti-mucin therapy.

In a further embodiment, the EphA2 agonistic agents of the invention are administered in combination with EphA4 agonistic agents (see U.S. Provisional Patent Application No. 60/476,909, filed Jun. 6, 2003, and U.S. Provisional Patent Application No. 60/503,356, filed Sep. 16, 2003, each of which is hereby incorporated by reference in its entirety).

5.4.2.1 Immunomodulatory Agents

In certain embodiments, the present invention provides compositions comprising one or more EphA2 agonistic agents of the invention and one or more immunomodulatory agents (i.e., agents which modulate the immune response in a subject), and methods for treating disorder involving hyperproliferative cells (e.g., epithelial or endothelial cells) in a subject comprising the administration of said compositions or administration of an EphA2-based prophylactic/therapeutic in combination with one or more immunomodulatory agents. In a specific embodiment of the invention, the immunomodulatory agent inhibits or suppresses the immune response in a human subject. Immunomodulatory agents are well-known to one skilled in the art and can be used in the methods and compositions of the invention.

Immunomodulatory agents can affect one or more or all aspects of the immune response in a subject. Aspects of the immune response include, but are not limited to, the inflammatory response, the complement cascade, leukocyte and lymphocyte proliferation, monocyte and/or basophil counts, and cellular communication among cells of the immune system. In certain embodiments of the invention, an immunomodulatory agent modulates one aspect of the immune response. In other embodiments, an immunomodulatory agent modulates more than one aspect of the immune response. In a preferred embodiment of the invention, the administration of an immunomodulatory agent to a subject inhibits or reduces one or more aspects of the subject's immune response capabilities.

In accordance with the invention, one or more immunomodulatory agents can be administered to a subject with a non-neoplastic hyperproliferative cell disorder prior to, subsequent to, or concomitantly with an EphA2 agonistic agent of the invention. Preferably, one or more immunomodulatory agents are administered to a subject with a non-neoplastic hyperproliferative cell or excessive cell accumulation disorder to reduce or inhibit one or more aspects of the immune response as necessary. Any technique well-known to one skilled in the art can be used to measure one or more aspects of the immune response, and thereby determine when it is necessary to administer an immunomodulatory agent. In a preferred embodiment, one or more immunomodulatory agents are administered to a subject with a non-neoplastic hyperproliferative cell or excessive cell accumulation disorder so as to transiently reduce or inhibit one or more aspects of the immune response. Such a transient inhibition or reduction of one or more aspects of the immune system can last for hours, days, weeks, or months. The transient reduction or inhibition of one or more aspects of the immune response potentiates the therapeutic effect of the EphA2 agonistic agent of the invention.

In a preferred embodiment, the immunomodulatory agent decreases the amount of IL-9. In a more preferred embodiment, the immunomodulatory agent is an antibody (preferably a monoclonal antibody) or fragment thereof that immunospecifically binds to IL-9 (see e.g., U.S. patent application Ser. No. ______ filed Apr. 12, 2004 entitled “Methods of Preventing or Treating Respiratory Conditions” by Reed (Attorney Docket No. 10271-113-999), U.S. patent application Ser. No. ______ filed Apr. 12, 2004 entitled “Recombinant IL-9 Antibodies and Uses Thereof” by Reed (Attorney Docket No. 10271-112-999), and U.S. patent application Ser. No. ______ filed Apr. 12, 2004 entitled “Anti-IL-9 Antibody Formulations and Uses Thereof” by Reed (Attorney Docket No. 10271-126-999), all of which are incorporated by reference herein in their entireties. Although not intending to be bound by a particular mechanism of action, the use of anti-IL-9 antibodies neutralizes IL-9's biological effect and, thereby, blocks or decreases inflammatory cell recruitment, epithelial or neointimal hyperplasia, and mucin production of epithelial cells.

In other embodiments, other immunomodulatory agents which can be used in the compositions and methods of the invention can be those that are commercially available and known to function as immunomodulatory agents. The immunomodulatory agents include, but are not limited to, agents such as cytokines, antibodies (e.g., human, humanized, chimeric, monoclonal, polyclonal, Fvs, sFvs, Fab or F(ab)₂ fragments or epitope binding fragments), inorganic compounds, or peptide mimetics. Further examples of immunomodulatory agents include, but are not limited to, anti-IL-13 monoclonal antibodies, anti-IL-4 monoclonal antibodies, anti-IL-5 monoclonal antibodies, anti-IL-2R antibodies (e.g., anti-Tac monoclonal antibody and BT 536), anti-CD4 monoclonal antibodies, anti-CD3 monoclonal antibodies, the anti-CD3 monoclonal human antibody OKT3, anti-CD8 monoclonal antibodies, anti-CD40 ligand monoclonal antibodies, anti-CD2 monoclonal antibodies, CTLA4-immunoglobulin, cyclophosphamide, cyclosporine A, macrolide antibiotics (e.g., FK506 (tacrolimus)), methylprednisolone (MP), corticosteroids, mycophenolate mofetil, rapamycin (sirolimus), mizoribine, deoxyspergualin, brequinar, malononitriloamindes (e.g., leflunamide), beta 2-agonists, leukotriene antagonists, and agents that decrease IgE levels.

The immunomodulatory activity of an immunomodulatory agent can be determined in vitro and/or in vivo by any technique well-known to one skilled in the art, including, e.g., by CTL assays, proliferation assays, immunoassays (e.g. ELISAs) for the expression of particular proteins such as co-stimulatory molecules and cytokines, and FACS.

5.4.2.2. Anti-Virals

In certain embodiments, the present invention provides compositions comprising one or more EphA2 agonistic agents of the invention and one or more anti-viral agents, and methods for treating disorder involving hyperproliferative cells in a subject comprising the administration of said compositions or administration of an EphA2-based prophylactic/therapeutic in combination with one or more anti-viral agents. In a specific embodiments of the invention, the disorder is a hyperproliferative epithelial cell disorder (e.g., asthma or COPD) and the anti-viral agent inhibits infection by a respiratory virus or inhibits or decreases replication of a respiratory virus. In specific embodiments, the respiratory virus is Respiratory Syncytial Virus (RSV), Human Metapneumovirus (HMPV), or Parainfluenza Virus (PIV). Anti-viral agents that are well-known to one skilled in the art and can be used in the methods and compositions of the invention. In a specific embodiment, the EphA2-based-antiviral prophylactic/therapeutic agents are administered to a patient that is a human child, infant, or an infant born prematurely who is currently infected with or has had a respiratory viral infection. Patients who have been infected with a respiratory viral infection (e.g., RSV) as infants, especially infants born prematurely, are at greater risk of developing asthma and/or COPD (see e.g., Zhoa et al., 2002, Pediatr. Allergy Immunol. 13:47-50; Message and Johnston, 2002, Br. Med. Bull. 61:29-43; Klinnert et al., 2001, Pediatrics 108:E69; Sigurs, 2002, Respiratory Res. 4:S8-S14).

In a preferred embodiment, the anti-viral RSV agent is one or more anti-RSV monoclonal antibodies. Anti-RSV-antigen antibodies that can be used with the methods of the invention bind immunospecifically to an antigen of RSV. In certain embodiments, the anti-RSV-antigen antibody binds immunospecifically to an RSV antigen of the Group A of RSV. In certain embodiments, the anti-RSV-antigen antibody binds immunospecifically to an RSV antigen of the Group B of RSV. In certain embodiments, an antibody binds to an antigen of RSV of one Group and cross reacts with the analogous antigen of the other Group.

In certain embodiments, an anti-RSV-antigen antibody binds immunospecifically to a RSV nucleoprotein, RSV phosphoprotein, RSV matrix protein, RSV small hydrophobic protein, RSV RNA-dependent RNA polymerase, RSV F protein, and/or RSV G protein.

In certain embodiments, an anti-RSV-antigen antibody binds to allelic variants of a RSV nucleoprotein, a RSV nucleocapsid protein, a RSV phosphoprotein, a RSV matrix protein, a RSV attachment glycoprotein, a RSV fusion glycoprotein, a RSV nucleocapsid protein, a RSV matrix protein, a RSV small hydrophobic protein, a RSV RNA-dependent RNA polymerase, a RSV F protein, a RSV L protein, a RSV P protein, and/or a RSV G protein.

It should be recognized that antibodies that immunospecifically bind to a RSV antigen are known in the art. For example, palivizumab is a humanized monoclonal antibody presently used for the prevention of RSV infection in pediatric patients. In a specific embodiment, an antibody to be used with the methods of the present invention is palivizumab, A4B4 (see e.g., International Application Publication No.: WO 02/43660) or an antigen-binding fragment thereof (e.g., contains one or more complementarity determining regions (CDRs) and preferably, the variable domain of palivizumab or A4B4). The amino acid sequence of palivizumab and A4B4 are disclosed, e.g., in Johnson et al., 1997, J. Infectious Disease 176:1215-1224, and U.S. Pat. No. 5,824,307; International Application Publication No.: WO 02/43660, entitled “Methods of Administering/Dosing Anti-RSV Antibodies for Prophylaxis and Treatment”, by Young et al.; and U.S. Provisional Patent Application 60/368,729 filed Mar. 29, 2002, which are incorporated herein by reference in their entireties.

In certain embodiments, the one or more anti-RSV-antigen antibodies include, but are not limited to, palivizumab or A4B4. In certain embodiments, the one or more antibodies or antigen-binding fragments thereof that bind immunospecifically to a RSV antigen comprise a Fc domain with a higher affinity for the FcRn receptor than the Fc domain of palivizumab or A4B4. Such antibodies are described in U.S. patent application Ser. No. 10/020,354, filed Dec. 12, 2001, which is incorporated herein by reference in its entireties. In certain embodiments, the one or more anti-RSV-antigen antibodies include, but are not limited to, AFFF, P12f2, P12f4, P11d4, Ale109, A12a6, A13c4, A17d4, A8c7, IX-493L1FR, H3-3F4, M3H9, Y10H6, DG, AFFF(1), 6H8, A8C7, L1-7E5, L2-15B10, A13a11, A1H5, A4B4(1), A4B4L1FR-S28R, or A4B4-F52S. These antibodies are disclosed in International Application Publication No.: WO 02/43660, entitled “Methods of Administering/Dosing Anti-RSV Antibodies for Prophylaxis and Treatment”, by Young et al., and U.S. patent application Ser. No. 10/628,088 filed Jul. 25, 2003, entitled “Methods of Treating and Preventing RSV, HMPV, and PIV Using Anti-RSV, Anti-HMPV, and Anti-PIV Antibodies”, and U.S. patent application Ser. No. 10/403,180 filed Mar. 31, 2003 entitled “Methods Of Administering/Dosing Anti-Rsv Antibodies For Prophylaxis And Treatment” which are incorporated herein by reference in their entireties.

In certain embodiments, the one or more antibodies that bind to a RSV antigen has a higher avidity and/or affinity for a RSV antigen than palivizumab or A4B4 has for the RSV F glycoprotein. In certain embodiments, the one or more antibodies that bind immunospecifically to a RSV antigen has a higher affinity and/or avidity for a RSV antigen than any previously known anti-RSV-antigen specific antibodies or antigen-binding fragments thereof. In certain embodiments, anti-RSV-antigen antibody is not palivizumab or A4B4.

In certain embodiments, the antibodies to be used with the methods and compositions of the invention or fragments thereof bind immunospecifically to one or more RSV antigens regardless of the strain of RSV. In particular, the anti-RSV-antigen antibodies bind to an antigen of human RSV A and human RSV B. In certain embodiments, the anti-RSV-antigen antibodies bind to RSV antigens from one strain of RSV versus another RSV strain. In particular, the anti-RSV-antigen antibody binds to an antigen of human RSV A and not to human RSV B or vice versa. In a specific embodiment, the antibodies or antigen-binding fragments thereof immunospecifically bind to the RSV F glycoprotein, G glycoprotein or SH protein. In certain embodiments, the anti-RSV-antigen antibodies bind immunospecifically to the RSV F glycoprotein. In another preferred embodiment, the anti-RSV-antigen antibodies or antigen-binding fragments thereof bind to the A, B, C, I, II, IV, V, or VI antigenic sites of the RSV F glycoprotein (see, e.g., López et al., 1998, J. Virol. 72:6922-6928, which is incorporated herein by reference in its entirety).

In certain embodiments, the anti-RSV-antigen antibodies are the anti-RSV-antigen antibodies of or are prepared by the methods of U.S. application Ser. No. 09/724,531, filed Nov. 28, 2000; Ser. No. 09/996,288, filed Nov. 28, 2001; and Ser. No. 09/996,265, filed Nov. 28, 2001, all entitled “Methods of Administering/Dosing Anti-RSV Antibodies for Prophylaxis and Treatment”, by Young et al., which are incorporated by reference herein in their entireties. Methods and composition for stabilized antibody formulations that can be used in the methods of the present invention are disclosed in U.S. Provisional Application Nos.: 60/388,921, filed Jun. 14, 2002, and 60/388,920, filed Jun. 14, 2002, which are incorporated by reference herein in their entireties.

In other embodiments, the anti-viral agent administered in combination with the agent of the invention decreases or inhibits the replication of HMPV and/or PIV. For examples of such agents and methods of treatment see U.S. patent application Ser. No. 10/628,088 filed Jul. 25, 2003, entitled “Methods of Treating and Preventing RSV, HMPV, and PIV Using Anti-RSV, Anti-HMPV, and Anti-PIV Antibodies” which is incorporated herein by reference in its entirety.

5.4.3 Conjugated Antibodies

The present invention encompasses the use of an antibody to target a prophylactic/therapeutic agent to cells involved in the non-neoplastic hyperproliferative disorder to be treated (e.g., hyperproliferating epithelial or endothelial cells). Such prophylactic/therapeutic agents are recombinantly fused or chemically conjugated (including both covalent and non-covalent conjugations) to an antibody or a fragment thereof (e.g., Fab fragment, Fd fragment, Fv fragment, F(ab)2 fragment, or portion thereof). In one embodiment, an EphA2 agonistic antibody of the invention or fragment thereof is conjugated to a prophylactic/therapeutic agent used to treat the non-neoplastic hyperproliferative disorder. Such prophylactic/therapeutic agents can be EphA2-based (e.g., agonistic agents of the invention) or non-EphA2-based (e.g., non-EphA2-based agents currently known to treat a non-neoplastic hyperproliferative cell or excessive cell accumulation disorder, an immunomodulatory agent, an anti-viral agent that decreases the replication of a respiratory virus, a bronchodilator, or an anti-mucin therapy).

An antibody or fragment thereof may be conjugated to a prophylactic/therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine-platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, an antibody or fragment thereof may be conjugated to a prophylactic/therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Patent Publication No. WO 97/33899), AIM II (see, International Patent Publication No. WO 97/34911), Fas Ligand (Takahashi et al., 1994, J Iminunol., 6:1567), and VEGI (see, International Patent Publication No. WO 99/23105), a thrombotic agent or an anti-angiogenic agent, e.g., angiostatin or endostatin; or, a biological response modifier such as, for example, a lymphokine (e.g., interleukin-1 (IL-1), interleukin-2 (IL-2), interleukin-6 (IL-6), granulocyte macrophage colony stimulating factor (GM-CSF), and granulocyte colony stimulating factor (G-CSF)), or a growth factor (e.g., growth hormone (GH)).

Moreover, an antibody can be conjugated to prophylactic/therapeutic moieties such as a radioactive materials or macrocyclic chelators useful for conjugating radiometal ions. In certain embodiments, the macrocyclic chelator is 1,4,7,10-tetraazacyclododecane-N,N′,N″,N′-tetraacetic acid (DOTA) which can be attached to the antibody via a linker molecule. Such linker molecules are commonly known in the art and described in Denardo et al., 1998, Clin. Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

In another embodiment, an antibody or fragment thereof that targets to the epithelial or endothelial cells affected by the non-neoplastic hyperproliferative disorder (e.g., through recognition of a pathology-associated marker) but does not immunospecifically bind EphA2 is conjugated to a prophylactic/therapeutic agent used to treat the non-neoplastic hyperproliferative disorder. Such prophylactic/therapeutic agents are EphA2-based (e.g., agonistic agents of the invention).

A conjugated agent's relative efficacy in comparison to the free agent can depend on a number of factors. For example, rate of uptake of the antibody-agent into the cell (e.g., by endocytosis), rate/efficiency of release of the agent from the antibody, rate of export of the agent from the cell, etc. can all effect the action of the agent. Antibodies used for targeted delivery of agents can be assayed for the ability to be endocytosed by the relevant cell type (i.e., the cell type associated with the disorder to be treated) by any method known in the art. Additionally, the type of linkage used to conjugate an agent to an antibody should be assayed by any method known in the art such that the agent action within the target cell is not impeded.

In another embodiment, antibodies can be fused or conjugated to liposomes, wherein the liposomes are used to encapsulate therapeutic agents (see e.g., Park et al., 1997, Can. Lett. 118:153-160; Lopes de Menezes et al., 1998, Can. Res. 58:3320-30; Tseng et al., 1999, Int. J. Can. 80:723-30; Crosasso et al., 1997, J. Pharm. Sci. 86:832-9). In a preferred embodiment, the pharmokinetics and clearance of liposomes are improved by incorporating lipid derivatives of PEG into liposome formulations (see e.g., Allen et al., 1991, Biochem Biophys Acta 1068:133-41; Huwyler et al., 1997, J. Pharmacol. Exp. Ther. 282:1541-6).

Therapeutic/prophylactic agents can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv. Drug Deliv. Rev. 53:171-216). Additional techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58. Methods for fusing or conjugating antibodies to polypeptide agents are known in the art. See, e.g., U.S. Pat. Nos. 5,336,603, 5,622,929, 5,359,046, 5,349,053, 5,447,851, and 5,112,946; EP 307,434; EP 367,166; International Patent Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341. Methods for fusing or conjugating antibodies to conjugated to another antibody are described by Segal in U.S. Pat. No. 4,676,980. The fusion of an antibody to a agent does not necessarily need to be direct, but may occur through linker sequences. Such linker molecules are commonly known in the art and described in Denardo et al., 1998, Clin Cancer Res. 4:2483-90; Peterson et al., 1999, Bioconjug. Chem. 10:553; Zimmerman et al., 1999, Nucl. Med. Biol. 26:943-50; Garnett, 2002, Adv. Drug Deliv. Rev. 53:171-216.

In other embodiments, antibody properties can be altered as desired (e.g., antibodies or fragments thereof with higher affinities and lower dissociation rates) through the techniques of gene-shuffling, motif-shuffling, exon-shuffling, and/or codon-shuffling (collectively referred to as “DNA shuffling”). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308. Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions immunospecifically bind to an antigen expressed on a cell associated with a particular disorder may be recombined with one or more components, motifs, sections, parts, domains, fragments, etc. of one or more heterologous molecules.

In other embodiments, the conjugated antibodies or fragments thereof can be additionally fused to marker sequences, such as a peptide, to facilitate purification. In preferred embodiments, the marker amino acid sequence is a hexa-histidine peptide, such as the tag provided in a pQE vector (QIAGEN, Inc., Chatsworth, Calif.), among others, many of which are commercially available (see e.g., Gentz et al., 1989, PNAS 86:821). Other peptide tags useful for purification include, but are not limited to, the hemagglutinin (HA) tag, which corresponds to an epitope derived from the influenza hemagglutinin protein (Wilson et al., 1984, Cell 37:767) and the “flag” tag. Any purification method known in the art can be used (see e.g., International Patent Publication WO 93/21232; EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452).

In other embodiments, conjugated antibodies or fragments or variants thereof can be conjugated to a diagnostic or detectable agent either alone or in combination with a prophylactic/therapeutic agent. Such antibodies can be useful for monitoring or prognosing the development or progression of a non-neoplastic hyperproliferative disorder as part of a clinical testing procedure, such as determining the efficacy of a particular therapy. Such diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹ Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰y), zinc (⁶⁵Zn); positron emitting metals using various positron emission tomographies, and nonradioactive paramagnetic metal ions.

5.5 Identification of EphA2 Agonistic Agents of the Invention

The invention provides methods of assaying and screening for EphA2 agonistic agents of the invention by incubating agents with cells that express EphA2, particularly epithelial or endothelial cells, and then assaying for increases EphA2 cytoplasmic tail phosphorylation, increased EphA2 degradation, increased EphA2 autophosphorylation, reduced EphA2 activity (other than autophosphorylation), decreased pathology-causing cell phenotype thereby identifying an EphA2 agonistic agent of the invention. In preferred embodiments, the EphA2 agonistic agent is an antibody, preferably monoclonal, which preferably has a low K_{off} rate (e.g., K_{off} less than 3×10⁻³ s⁻¹). The invention also encompasses the use of in vivo assays to identify EphA2 agonistic agents, e.g., by reduction in pathological symptoms and/or decreased amount of pathology-associated molecules (e.g., mucin, inflammatory molecules or extracellular matrix molecules).

5.5.1 Agents that Increase EphA2 Cytoplasmic Tail Phosphorylation

The invention provides methods of assaying and screening for EphA2 agonistic agents that increase EphA2 phosphorylation and/or EphA2 degradation when contacting cells expressing EphA2, particularly epithelial or endothelial cells. Any method known in the art to assay either the level of EphA2 phosphorylation or expression can be used to assay candidate EphA2 agents to determine their activity (see, e.g., Section 6.3.1, infra).

5.5.2 Agents that Inhibit Pathology-Causing Epithelial or Endothelial Cell Phenotypes

EphA2 agonistic agents of the invention may reduce (and preferably inhibit) pathology-causing epithelial or endothelial cell phenotypes, for example, mucin secretion, differentiation into mucin-secreting cells, secretion of inflammatory factors, secretion of ECM factors, particularly fibronectin, and/or hyperproliferation. One of skill in the art can assay candidate EphA2 agonistic agents for their ability to inhibit such behavior.

In some embodiments, in vitro models of lung epithelia can be used to screen candidate agents. Cells can be cultured to form a pseudo-stratified, highly differentiated model tissue from human-derived tracheal/bronchial epithelial cells (e.g., NHBE or TBE cells) which closely resembles the epithelial tissue of the respiratory tract. The cultures can be grown on cell culture inserts at the air-liquid interface, allowing for gas phase exposure of volatile materials in airway inflammation and irritancy studies, as well as in inhalation toxicity studies. Transepithelial permeability can be measured for inhaled drug delivery studies. Such model systems are available commercially such as EpiAirway™ Tissue Model System (MatTek Corp., Ashland, Mass.).

Mucin Secretion

In one embodiment, the pathology-causing epithelial cell phenotype is mucin secretion. Candidate EphA2 agonistic agents can be assayed for their ability to decrease or inhibit mucin secretion by a number of in vitro and in vivo assays. One example of an in vitro assay that can be used to measure mucin release from cultured airway goblet cells is a hamster tracheal surface epithelial (HTSE) cell culture system (see U.S. Pat. No. 6,245,320). Briefly, tracheas obtained from 7-8 week old male Golden Syrian hamsters (Harlan Sprague Dawley, Indianapolis, Ind.) are used to harvest HTSE cells. HTSE cells are then cultured on a collagen gel as described in Kim et al., 1989, Exp. Lung Res. 15:299-314. Mucins are metabolically radiolabeled by incubating confluent cultures with labeling medium for 24 hours as described in Kim et al., 1989, Am. J Resp. Cell Mol. Biol. 1:137-143. At the end of the 24 hour incubation period, the spent media (the pretreatment sample) is collected, and the labeled cultures are washed twice with PBS without Ca⁺⁺ and Mg⁺⁺ and then chased for 30 min in the presence of candidate EphA2 agonistic agents. The chased media are referred to as the treatment samples. At the end of the chase period, floating cells and cell debris are removed from the treatment samples by centrifugation and assayed for their labeled mucin content. High molecular weight glycoconjugates that are excluded after Sepharose CL-4B (Pharmacia, Upsaala, Sweden) gel-filtration column chromatography and that are resistant to hyaluronidase are defined as mucins (see Kim et al., 1985, J. Biol. Chem. 260:4021:4027). Mucins are then measured by column chromatography as described in Kim et al., 1987, PNAS 84:9304-9308. The amount of secreted mucin in HTSE cultures before and after incubation with a candidate EphA2 agonistic agent can be determined.

Other in vitro assays can be used, such as primary tracheal epithelial cell cultures maintained in an air/liquid interface system that maintains differentiated characteristics (Adler et al., 1992, Am. J. Respir. Cell Mol. Biol. 6:550-556) and lung epithelial cell lines (e.g., NIH-292 cells). Standard molecular biological techniques can be use to determine mucin amount, including but not limited to, western blot and ELISA for protein expression levels and PCR and northern blots for RNA expression levels.

In vivo assays can also be used to identify EphA2 agonistic agents of the invention. Animal models for asthma or COPD can also be used to identify EphA2 agonistic agents of the invention. For example, a murine model of endotoxin/LPS-induced lung inflammation can be used to assay the affect of candidate EphA2 agonistic agents on differentiation of mucin-secreting cells (Steiger et al., 1995, J. Am. Respir. Cell Mol. Biol., 12:307-14 and U.S. Pat. No. 6,083,973). Briefly, lung inflammation can be induced in mice or rats by repeated instillation of LPS (LPS derived from Pseudomonas aeriginos; Sigma Chemical) 400 μg/kg/dose/day for three days. Animals can be treated with a candidate EphA2 agonistic agent once daily, starting 24 hours prior to the first LPS challenge. Animals are sacrificed 24 hours after the last LPS challenge by exsanguination under deep anesthesia. The lungs are lavaged with phosphate buffered saline (2×5 ml) to wash out mucous layer. The bronchial lavage fluid is centrifuged for 10 min and the cell-free supernate is frozen and stored −20° C. until analysis to determine the amount of mucin present. Amount of mucin secretion can be determined by any method known in the art, e.g., by dot blot assay using Alcian-blue and/or periodic acid-Schiff stains or by western blot/ELISA analysis using anti-mucin antibodies.

Other animal models of asthma/COPD can also be used to identify EphA2 agonistic agents of the invention such as mice that overexpress IL-4 (Temann et al., 1997, Am. J. Respir. Cell Mol. Biol. 16:471-8), IL-13 (Kuperman, et al., 2002, Nat. Med. July 1, epub ahead of print) or IL-9 either systemically or only in lung tissue. Reduction in pathological symptoms can be used to identify EphA2 agonistic agents of the invention as well as a decreased amount of mucin present in bronchial lavage fluid or induced sputum samples (Fahy et al., 1993, Am. Rev. Respir. Dis. 147:1132-1137). Another example of an animal model is the murine adoptive transfer model in which aeroallergen provocation of TH1 or TH2 recipient mice results in TH effector cell migration to the airways and is associated with an intense neutrophilic (TH1) and eosinophilic (TH2) lung mucosal inflammatory response (Cohn et al., 1997, J. Exp. Med. 1861737-1747). For a review of animal models of COPD see Szelenyi and Marx, 2001, Arzneimittelforschung 51:1004-14.

Differentiation into Mucin-Secreting Cells

In one embodiment, the pathology-causing epithelial cell phenotype is differentiation into mucin-secreting cells (e.g., goblet cells). Candidate EphA2 agonistic agents can be assayed (both in vitro and in vivo) for their ability to decrease or inhibit epithelial cell differentiation to mucin-secreting cells. Animal models for asthma or COPD can be used to identify EphA2 agonistic agents of the invention. For example, animals with LPS-induced lung inflammation can be used to assay the affect of candidate EphA2 agonistic agents on differentiation of mucin-secreting cells (see U.S. Pat. No. 6,083,973). Animals with LPS-induced lung inflammation that were either treated with a candidate EphA2 agonistic agent or were an untreated control are sacrificed before lung perfusion with 10% neutral buffered formalin by intratracheal instillation at a constant rate (5 ml at 1 ml/min). The lung lobes are then excised and immersed in fixative for 24 hours prior to processing. Standard methods can be used to prepare 5 μm paraffin sections. Sections are stained with Alcian blue (pH 2.5) and/or periodic acid/Schiffs reagent and/or anti-mucin antibodies to detect mucosubstances within the lung tissue. Morphometric analysis for goblet hyperplasia can performed by counting all airways ≧2 mm in diameter and determining the percentage of airways that contain positively stained cells.

Secretion of Inflammatory Factors

In one embodiment, the pathology-causing epithelial or endothelial cell phenotype is secretion of inflammatory factors. Although mast cells and eosinophils may initially release mediators of the inflammatory response, epithelial cells in hyperproliferative disorders do alter their phenotype to one that secretes cytokines and chemokines (Holgate et al., 1999, Clin. Exp. Allergy 29:90-5). Any method known in the art to assay for cytokine/chemokine production or secretion can be used to quantitate differences in in vitro or in vivo epithelial or endothelial cells that have been either treated or untreated with candidate EphA2 agonistic agents. In certain embodiments, IL-4, IL-9, and/or IL-13 production or secretion are assessed.

Non-Neoplastic Hyperproliferation

In one embodiment, the pathology-causing epithelial or endothelial cell phenotype is non-neoplastic hyperproliferation. Many assays well-known in the art can be used to assess survival, growth and/or proliferation; for example, cell proliferation can be assayed by measuring (³H)-thymidine incorporation, by direct cell count, by detecting changes in transcription, translation or activity of known genes such as cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as western blotting or immunoprecipitation using commercially available antibodies (for example, many cell cycle marker antibodies are from Santa Cruz Inc.). mRNA can be quantitated by methods that are well known and routine in the art, for example by northern analysis, RNase protection, the polymerase chain reaction in connection with the reverse transcription, etc. Cell viability can be assessed by using trypan-blue staining or other cell death or viability markers known in the art.

The present invention provides for cell cycle and cell proliferation analysis by a variety of techniques known in the art, including but not limited to the following:

As one example, bromodeoxyuridine (BRDU) incorporation may be used as an assay to identify proliferating cells. The BRDU assay identifies a cell population undergoing DNA synthesis by incorporation of BRDU into newly synthesized DNA. Newly synthesized DNA may then be detected using an anti-BRDU antibody (see Hoshino et al., 1986, Int. J. Cancer 38:369; Campana et al., 1988, J. Immunol. Meth. 107:79).

Cell proliferation may also be examined using (³H)-thymidine incorporation (see e.g., Chen, 1996, Oncogene 13:1395-403; Jeoung, 1995, J. Biol. Chem. 270:18367-73). This assay allows for quantitative characterization of S-phase DNA synthesis. In this assay, cells synthesizing DNA will incorporate (³H)-thymidine into newly synthesized DNA. Incorporation may then be measured by standard techniques in the art such as by counting of radioisotope in a Scintillation counter (e.g. Beckman LS 3800 Liquid Scintillation Counter).

Detection of proliferating cell nuclear antigen (PCNA) may also be used to measure cell proliferation. PCNA is a 36 kilodalton protein whose expression is elevated in proliferating cells, particularly in early G1 and S phases of the cell cycle and therefore may serve as a marker for proliferating cells. Positive cells are identified by immunostaining using an anti-PCNA antibody (see L1 et al., 1996, Curr. Biol. 6:189-99; Vassilev et al., 1995, J. Cell Sci. 108:1205-15).

Cell proliferation may be measured by counting samples of a cell population over time (e.g. daily cell counts). Cells may be counted using a hemacytometer and light microscopy (e.g. HyLite hemacytometer, Hausser Scientific). Cell number may be plotted against time in order to obtain a growth curve for the population of interest. In a preferred embodiment, cells counted by this method are first mixed with the dye Trypan-blue (Sigma), such that living cells exclude the dye, and are counted as viable members of the population.

DNA content and/or mitotic index of the cells may be measured, for example, based on the DNA ploidy value of the cell. For example, cells in the G1 phase of the cell cycle generally contain a 2N DNA ploidy value. Cells in which DNA has been replicated but have not progressed through mitosis (e.g. cells in S-phase) will exhibit a ploidy value higher than 2N and up to 4N DNA content. Ploidy value and cell-cycle kinetics may be further measured using propidum iodide assay (see e.g. Turner, et al., 1998, Prostate 34:175-81). Alternatively, the DNA ploidy may be determined by quantitation of DNA Feulgen staining (which binds to DNA in a stoichiometric manner) on a computerized microdensitometrystaining system (see e.g., Bacus, 1989, Am. J. Pathol. 135:783-92). In an another embodiment, DNA content may be analyzed by preparation of a chromosomal spread (Zabalou, 1994, Hereditas. 120:127-40; Pardue, 1994, Meth. Cell Biol. 44:333-351).

The expression of cell-cycle proteins (e.g., CycA. CycB, CycE, CycD, cdc2, Cdk4/6, Rb, p21, p27, etc.) provide crucial information relating to the proliferative state of a cell or population of cells. For example, identification in an anti-proliferation signaling pathway may be indicated by the induction of p21^{cip1}. Increased levels of p21 expression in cells results in delayed entry into G1 of the cell cycle (Harper et al., 1993, Cell 75:805-816; L1 et al., 1996, Curr. Biol. 6:189-199). p21 induction may be identified by immunostaining using a specific anti-p21 antibody available commercially (e.g. Santa Cruz). Similarly, cell-cycle proteins may be examined by western blot analysis using commercially available antibodies. In another embodiment, cell populations are synchronized prior to detection of a cell cycle protein. Cell cycle proteins may also be detected by FACS (fluorescence-activated cell sorter) analysis using antibodies against the protein of interest.

EphA2 agonistic agents of the invention can also be identified by their ability to change the length of the cell cycle or speed of cell cycle so that cell proliferation is decreased or inhibited. In one embodiment the length of the cell cycle is determined by the doubling time of a population of cells (e.g., using cells contacted or not contacted with one or more candidate EphA2 agonistic agents). In another embodiment, FACS analysis is used to analyze the phase of cell cycle progression, or purify G1, S, and G2/M fractions (see e.g., Delia et al., 1997, Oncogene 14:2137-47).

5.5.3 Agents that Inhibit Pathology-Causing Endothelial Cell Phenotypes

EphA2 agonistic agents of the invention may preferably reduce (and preferably inhibit) pathology-causing endothelial cell phenotypes, for example, increased cell migration (not including metastasis), increased cell volume, secretion of extracellular matrix molecules (e.g., collagen, fibronectin, proteoglycans, etc.) or matrix metalloproteinases (e.g., gelatinases, collagenases, and stromelysins), and hyperproliferation. One of skill in the art can assay candidate EphA2 agonistic agents for their ability to inhibit such behavior.

Cell Migration

In one embodiment, the pathology-causing endothelial cell phenotype is increased cell migration (not including metastasis). Candidate EphA2 agonistic agents can be assayed (both in vitro and in vivo) for their ability to decrease or inhibit endothelial cell migration. Any assay known in the art can be used to measure endothelial cell migration. For example, migration can be evaluated in a Boyden chamber migration assay. Briefly, endothelial cells (e.g., smooth muscle cell) can be added to the upper well of the chamber. Following cell attachment, one or more candidate EphA2 agonistic agents can be added to the upper chamber. Cells can be allowed to migrate to the lower chamber either with or without an attracted (e.g., PDGF) added to the medium of the lower chamber. Cells which migrated through to the lower chamber can be stained and counted.

Secretion of Extracellular Matrix Molecules such as Fibronectin and Matrix Metalloproteinases

In one embodiment, the pathology-causing endothelial cell phenotype is secretion of extracellular matrix molecules, such as fibronectin, or matrix metalloproteinases. Any method known in the art to assay for extracellular matrix molecule and matrix metalloproteinase production or secretion can be used to quantitate differences in in vitro or in vivo endothelial cells that have been either treated or untreated with candidate EphA2 agonistic agents. For example, western or northern blot analysis, reverse transcription-polymerase chain reaction, or ELISA assays can be used to quantitate expression levels. The activity of matrix metalloproteinases can be assayed by any method known in the art including zymography (see e.g., Badier-Commander, 2000, J. Pathol. 192:105-112).

In one specific embodiment, the ability to decrease expression level and/or activity level of gelatinase-A (also known as MMP-2) is used to screen for EphA2 agonistic agents of the invention. In another embodiment, the ability to modulate fibronectin expression is used to screen for EphA2 agonistic agents of the invention.

Non-Neoplastic Hyperproliferation

In one embodiment, the pathology-causing endothelial cell phenotype is non-neoplastic hyperproliferation. Many assays well-known in the art can be used to assess survival, growth and/or proliferation. Any in vitro assay listed in Section 5.5 can be used to assess growth, proliferation and/or cell survival of endothelial cells in the presence and absence of candidate EphA2 agonistic agents. Animal models of endothelial cell hyperproliferation can also be used. For example, New Zealand White rabbits can be used for an in vivo model of restenosis (see e.g., Feldman et al, 2000, Circulation; 101:908-16; Feldman et al., 2001, Circulation 103:3117-22; Frederick et al., 2001, Circulation 104:3121-4). Briefly, bilateral iliac artery balloon angioplasty is performed with a 3-mm-diameter balloon (3×1-minute inflation, 10 atm); then a 15-mm-long Crown stent (Cordis) mounted over the balloon was implanted in the right iliac artery only (30-second inflation, 10 atm). Animals are euthanized at 1, 3, 7, 30, or 60 days after injury. At each time point, right (stent) and left (balloon angioplasty) iliac arteries were harvested, flushed with ice-cold saline, cleaned of any adipose tissue, and divided into 2 or 3 segments. Morphometric analyses and immunohistochemistry are performed on the excised arteries. Stented and nonstented arterial segments are fixed in 4% paraformaldehyde. Morphometric analyses are performed on hematoxylin-phloxin-safran-stained cross sections of the arteries. For immunohistochemistry, arterial segments are embedded in OCT compound, frozen in liquid nitrogen and chilled isopentane after stent struts are removed with microforceps. Four-micrometer cross sections are obtained from each block and immunostained, e.g., with anti extracellular matrix molecule or anti-matrix metalloproteinase antibodies.

5.5.4 Agents that Decrease EphA2 Activity

The invention provides methods of assaying and screening for EphA2 agonistic agents that decrease EphA2 activity (other than autophosphorylation). Ligand binding causes EphA2 autophosphorylation (R. A. Lindberg, et al., Molecular & Cellular Biology 10: 6316, 1990) and EphA2 activity causing EphA2 signaling. However, unlike other receptor tyrosine kinases, EphA2 retains activity in the absence of ligand binding or phosphotyrosine content (Zantek, et al, Cell Growth & Differentiation 10:629, 1999). In some embodiments, activity of both ligand bound or unbound EphA2 (other than autophosphorylation) is decreased by EphA2 agonistic agents of the invention.

In one embodiment, EphA2 activity of ligand bound EphA2 is decreased. Ligand-mediated EphA2 cytoplasmic tail phosphorylation has been shown to cause the EphA2 cytoplasmic tail to interact with the PTB and SH2 domains of SHC, promote nuclear translocation and phosphorylation of ERK kinases, and increase nuclear induction of the Elk-1 transcription factor (Pratt and Kinch, 2002, Oncogene 21:7690-9). EphA2 agonistic agents decrease ligand-mediated EphA2 signaling. In a specific embodiment, EphA2 agonistic agents decrease ligand-mediated EphA2 interaction with SHC. In another specific embodiment, EphA2 agonistic agents decrease ligand-mediated nuclear translocation and/or phosphorylation of ERK kinases. In another specific embodiment, EphA2 agonistic agents decrease ligand-mediated nuclear induction of the Elk-1 transcription factor. Any method in the art to assay ligand-mediated EphA2 signaling can be used to screen EphA2 agents to determine their ability to decrease ligand-mediated EphA2 signaling, e.g., reporter gene assay, immunoprecipitation, immunoblotting, GST fusion protein pull down assay (see, e.g., Pratt and Kinch, 2002, Oncogene 21:7690-9).

In another embodiment, EphA2 activity of EphA2 not bound to ligand is decreased. Such agonistic agents are identified by assaying for the ability of a candidate EphA2 agent to decrease the level of EphA2 activity that is present in an EphA2-expressing cell, particularly an epithelial cell or endothelial cell, when unbound to ligand. In some embodiments, the candidate agents are screened for ability to decrease EphA2 activity (e.g., in a kinase activity assay) that is present when EphA2 is not bound to ligand. In other embodiments, candidate agents are screened for the ability to decrease signaling through the EphA2 signaling cascade (e.g., in a reporter gene assay such as a CATalyse Reporter Gene Assay available from Serologicals Corporation, Norcross, Ga.) that is active when EphA2 is not bound to ligand.

5.5.5 Antibodies with Low K_{off} Rates

Antibodies of the invention that as immunospecifically bind to and agonize EphA2 receptor (i.e., increase EphA2 cytoplasmic tail phosphorylation, increase EphA2 degradation, increase EphA2 autophosphorylation, reduce EphA2 activity (other than autophosphorylation), decrease pathology-causing cell phenotype). Methods as discussed previously (see, e.g., Sections 5.5.1-5.5.4, supra) can be used to identify such antibodies of the invention. Additionally, EphA2 antibodies with low K_{off} rates can be used in the methods of the invention.

The binding affinity of a monoclonal antibody of the invention to EphA2 or a fragment thereof and the off-rate of a monoclonal antibody-EphA2 interaction can be determined by competitive binding assays. One example of a competitive binding assay is a radioimmunoassay comprising the incubation of labeled EphA2 (e.g., ³H or ¹²⁵I) with the monoclonal antibody of interest in the presence of increasing amounts of unlabeled EphA2, and the detection of the monoclonal antibody bound to the labeled EphA2. The affinity of a monoclonal antibody for an EphA2 and the binding off-rates can be determined from the data by scatchard plot analysis. Competition with a second monoclonal antibody can also be determined using radioimmunoassays. In this case, EphA2 is incubated with a monoclonal antibody conjugated to a labeled compound (e.g., ³H or ¹²⁵I in the presence of increasing amounts of a second unlabeled monoclonal antibody.

In a preferred embodiment, BIAcore kinetic analysis is used to determine the binding on and off rates of monoclonal antibodies to EphA2. BIAcore kinetic analysis comprises analyzing the binding and dissociation of a monoclonal antibody from chips with immobilized EphA2 or fragment thereof on their surface.

An antibody that immunospecifically binds EphA2 preferably has a K_{off} rate 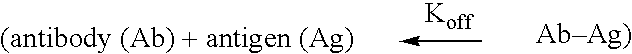
of less than 3×10⁻³ s⁻¹, less than 10⁻³ s⁻¹, less than 10⁻⁴ s, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹.

5.6 Characterization and Demonstration of Therapeutic or Prophylactic Utility

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The anti-hyperproliferative cell or anti-excessive cell accumulation disorder activity of the therapies used in accordance with the present invention also can be determined by using various experimental animal models for the study of anti-hyperproliferative epithelial cell disorders and anti-hyperproliferative endothelial cell disorders.

5.6.1 Demonstration of Prophylactic/Therapeutic Utility

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which a patient tissue sample is grown in culture, and exposed to or otherwise administered a protocol, and the effect of such protocol upon the tissue sample is observed, e.g., increased EphA2 cytoplasmic tail phosphorylation, increased EphA2 autophosphorylation, reduced EphA2 activity (other than autophosphorylation), decreased a pathology-causing cell phenotype (e.g., decreased mucin secretion, decreased expression of mucin-secreting cell markers, decreased survival/proliferation of EphA2 expressing epithelial cells or endothelial cells, decreased cell migration (not including metastasis), decreased cell volume, and/or decreased secretion of inflammatory factors, extracellular matrix molecules or matrix metalloproteinases). A demonstration of any of the aforementioned properties of the contacted cells indicates that the therapeutic agent is effective to treat the condition in the patient. Alternatively, instead of culturing cells from a patient, therapeutic agents and methods may be screened using cells of a epithelial or endothelial cell line. Many assays standard in the art can be used to assess such parameters relevant to disorder etiology (see e.g., Section 5.5).

In some embodiments, where the disorder is a non-neoplastic hyperproliferative lung epithelial cell disorder, in vitro models of lung epithelia can be used to demonstrate prophylactic/therapeutic utility. Cells can be cultured to form a pseudo-stratified, highly differentiated model tissue from human-derived tracheal/bronchial epithelial cells (e.g., NHBE or TBE cells) which closely resembles the epithelial tissue of the respiratory tract. The cultures can be grown on cell culture inserts at the air-liquid interface, allowing for gas phase exposure of volatile materials in airway inflammation and irritancy studies, as well as in inhalation toxicity studies. Transepithelial permeability can be measured for inhaled drug delivery studies. Such model systems are available commercially such as EpiAirway™ Tissue Model System (MatTek Corp., Ashland, Mass.).

In other embodiments, the disorder is lung fibrosis and the in vitro model is Beas-2B cells (bronchial epithelium cells transformed with SV40 virus) treated with bleomycin. In another embodiment, an in vivo model for lung fibrosis is bleomycin treatment of susceptible strains of mice. Bleomycin induces lung epithelial cell death, followed by acute neutrophilic influx, subsequent chronic inflammation, and parenchymal fibrosis in mice. Bleomycin-treated lung epithelial cells as a model for lung fibrosis replicates key pathologic features of human lung fibrotic diseases such as IPF.

Compounds for use in therapy can be tested in suitable animal model systems prior to testing in humans, including but not limited to in rats, mice, chicken, cows, monkeys, rabbits, hamsters, etc., for example, the animal models described above. The compounds can then be used in the appropriate clinical trials.

Further, any assays known to those skilled in the art can be used to evaluate the prophylactic and/or therapeutic utility of the combinatorial therapies disclosed herein for treatment or prevention of a non-neoplastic hyperproliferative cell or excessive cell accumulation disorder.

5.6.2 Dosages

The amount of the composition of the invention which will be effective in the treatment, management, or prevention of non-neoplastic hyperproliferative cell or excessive cell accumulation disorders can be determined by standard research techniques. For example, the dosage of the composition which will be effective in the treatment, management, or prevention of a non-neoplastic hyperproliferative cell or excessive cell accumulation disorder can be determined by administering the composition to an animal model such as, e.g., the animal models known to those skilled in the art. In addition, in vitro assays may optionally be employed to help identify optimal dosage ranges.

Selection of the preferred effective dose can be determined (e.g., via clinical trials) by a skilled artisan based upon the consideration of several factors which will be known to one of ordinary skill in the art. Such factors include the disorder to be treated or prevented, the symptoms involved, the patient's body mass, the patient's immune status and other factors known by the skilled artisan to reflect the accuracy of administered pharmaceutical compositions.

The precise dose to be employed in the formulation will also depend on the route of administration, and the seriousness of the non-neoplastic hyperproliferative cell or excessive cell accumulation disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human and humanized antibodies have a longer half-life within the human body than antibodies from other species due to the immune response to the foreign polypeptides. Thus, lower dosages of human antibodies and less frequent administration is often possible.

For other therapeutic agents administered to a patient, the typical doses of various immunomodulatory agents, anti-viral agents that decreases the replication of a respiratory virus, bronchodilators, or anti-mucin therapies are known in the art. Given the invention, certain preferred embodiments will encompass the administration of lower dosages in combination treatment regimens than dosages recommended for the administration of single agents.

The invention provides for any method of administrating lower doses of known prophylactic or therapeutic agents than previously thought to be effective for the prevention, treatment, management, or prevention of a non-neoplastic hyperproliferative cell or excessive cell accumulation disorders. Preferably, lower doses of known therapies are administered in combination with lower doses of EphA2 agonistic agents of the invention.

5.7 Pharmaceutical Compositions

The compositions of the invention include bulk drug compositions useful in the manufacture of pharmaceutical compositions (e.g., impure or non-sterile compositions) and parenteral pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of one or more EphA2 agonistic agents of the invention and a pharmaceutically acceptable carrier. In a further embodiment, the composition of the invention further comprises an additional therapeutic, e.g., immunomodulatory or anti-viral agent.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known and can be used to administer an agonistic monoclonal antibody of the invention or the combination of an agonistic monoclonal antibody of the invention and a prophylactic agent or therapeutic agent useful for preventing or treating a non-neoplastic hyperproliferative cell or excessive cell accumulation disorder, e.g., encapsulation in liposomes, microparticles, microcapsules, recombinant cells capable of expressing the antibody or antibody fragment, receptor-mediated endocytosis (see, e.g., Wu and Wu, 1987, J. Biol. Chem. 262:4429-4432), construction of a nucleic acid as part of a retroviral or other vector, etc. Methods of administering a prophylactic or therapeutic agent of the invention include, but are not limited to, parenteral administration (e.g., intradermal, intramuscular, intraperitoneal, intravenous and subcutaneous), epidural, and mucosal (e.g., intranasal, inhaled, and oral routes). In a specific embodiment, prophylactic or therapeutic agents of the invention are administered intramuscularly, intravenously, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the prophylactic or therapeutic agent can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; International Patent Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Patent Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

5.7.1 Gene Therapy

In a specific embodiment, nucleic acids of the invention (e.g., EphA2 antisense nucleic acids, EphA2 dsRNA, EphA2 ribozymes, or nucleic acids that encode an EphA2 intrabody) are administered to treat, prevent or manage epithelial or endothelial cell hyperproliferation by way of gene therapy. Gene therapy refers to therapy performed by the administration to a subject of an expressed or expressible nucleic acid. In this embodiment of the invention, the nucleic acids are produce and mediate a prophylactic or therapeutic effect.

Any of the methods for gene therapy available in the art can be used according to the present invention. Exemplary methods are described below.

For general reviews of the methods of gene therapy, see Goldspiel et al., 1993, Clinical Pharmacy 12:488; Wu and Wu, 1991, Biotherapy 3:87; Tolstoshev, 1993, Ann. Rev. Pharmacol. Toxicol. 32:573; Mulligan, 1993, Science 260:926-932; and Morgan and Anderson, 1993, Ann. Rev. Biochem. 62:191; May, 1993, TIBTECH 11:155. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises a nucleic acid of the invention (e.g., encode an EphA2 antisense or intrabody molecule), said nucleic acid being part of an expression vector that expresses the nucleic acid in a suitable host. In particular, such nucleic acids have promoters, preferably heterologous promoters, said promoter being inducible or constitutive, and, optionally, tissue-specific. In another particular embodiment, nucleic acid molecules used comprise nucleic acid molecules of the invention flanked by regions that promote homologous recombination at a desired site in the genome, thus providing for intrachromosomal expression of the nucleic acids of the invention (Koller and Smithies, 1989, PNAS 86:8932; Zijlstra et al., 1989, Nature 342:435).

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy. In a specific embodiment, the nucleic acid sequences are directly administered in vivo. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see e.g., U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide, e.g., through a thioester bond, which is known to enter the cell (e.g., a membrane permeable sequence) and/or nucleus, by administering it in linkage to a ligand subject to receptor-mediated endocytosis (see, e.g., Wu and Wu, 1987, J. Biol. Chem. 262:4429) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., International Patent Publication Nos. WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, 1989, PNAS 86:8932; and Zijlstra et al., 1989, Nature 342:435).

In a specific embodiment, viral vectors that contain the nucleic acid sequences of the invention are used. For example, a retroviral vector can be used (see Miller et al., 1993, Meth. Enzymol. 217:581). These retroviral vectors contain the components necessary for the correct packaging of the viral genome and integration into the host cell DNA. The nucleic acid sequences to be used in gene therapy are cloned into one or more vectors, which facilitates delivery of the nucleic acid into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Adenoviruses have the advantage of being capable of infecting non-dividing cells. Kozarsky and Wilson, 1993, Current Opinion in Genetics Development 3:499 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431; Rosenfeld et al., 1992, Cell 68:143; Mastrangeli et al., 1993, J. Clin. Invest. 91:225; International Patent Publication No. WO94/12649; and Wang et al., 1995, Gene Therapy 2:775. In a preferred embodiment, adenovirus vectors are used. Adeno-associated virus (AAV) has also been proposed for use in gene therapy (Walsh et al., 1993, Proc. Soc. Exp. Biol. Med. 204:289-300; and U.S. Pat. No. 5,436,146).

Numerous techniques are known in the art for the introduction of foreign genes into cells (see, e.g., Loeffler and Behr, 1993, Meth. Enzymol. 217:599; Cohen et al., 1993, Meth. Enzymol. 217:618) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. The amount of cells envisioned for use depends on the desired effect, patient state, etc., and can be determined by one skilled in the art.

5.8 Kits

The invention provides a pharmaceutical pack or kit comprising one or more containers filled with an EphA2 agonistic agent of the invention. Additionally, one or more other prophylactic or therapeutic agents useful for the treatment of a non-neoplastic hyperproliferative cell or excessive cell accumulation disorder or other relevant agent (e.g., an immunomodulatory agent and/or an anti-viral agent) can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises one or more a monoclonal antibodies of the invention. In another embodiment, a kit further comprises one or more other prophylactic or therapeutic agents useful for the treatment of a hyperproliferative epithelial disorder, in one or more containers. Preferably the monoclonal antibody of the invention is Eph099B-102.147, Eph099B-208.261, Eph099B-210.248, B233, EA2, or EA5. In certain embodiments, the other prophylactic or therapeutic agent is an immunomodulatory agent (e.g., anti-IL-9 antibody). In other embodiments, the prophylactic or therapeutic agent is an anti-viral agent (e.g., anti-RSV agent).

### 6. EXAMPLE

6.1. EGF Increases EphA2 Expression

HMT-3522 cells, variant S1 (a non-tumorigenic immortalized epithelial cell line), were treated with exogenous EGF, and EphA2 levels were determined. Quantitative RT-PCR was performed to determine mRNA expression levels in both untreated and EGF-treated cells. mRNA levels of the housekeeping gene glyceraldehyde-3-phosphate dehydrogenase (GADPH) were also determined and used as a control. Primers and PCR conditions used to amplify EphA2 and GAPDH were as follows:

| | |
|---|---|
| EPHA2 | |
| 5′ ATG GAG CTC CAG GCA GCC CGC 3′ | (SEQ ID NO: 40) |
| 5′ GCC ATA CGG GTG TGT GAG CCA | (SEQ ID NO: 41) |
| GC 3′ | |
| GAPDH | |
| 5′ CAG TGG TGG ACC TGA CCT GCC | (SEQ ID NO: 42) |
| GTC T 3′ | |
| 5′ CTC AGT GTA GCC CAG GAT GCC | (SEQ ID NO: 43) |
| CTT GAG 3′ | |

PCR reactions (50 μl total volume) were incubated at 94° C. for 2 min before cycling at 94° C. for 1 min/60° C. for 1 min/72° C. for 1 min thirty five times. Samples were then incubated at 72° C. for 10 min. EphA2 primers yielded a 150 bp product while GAPDH primers yielded a 104 bp product.

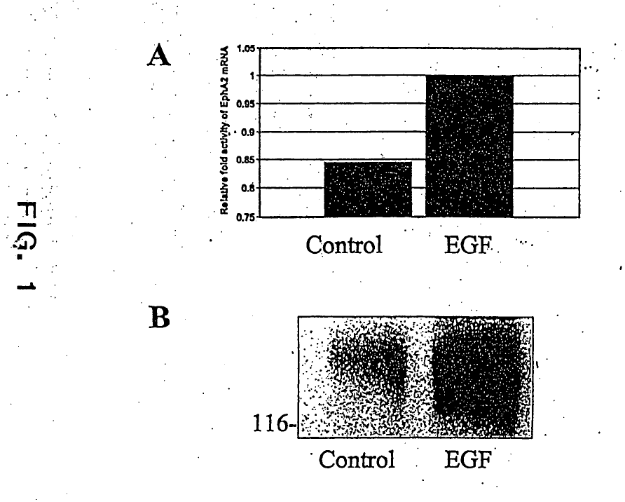

The level of EphA2 mRNA in EGF-treated cells was defined as 1. Untreated control cells expressed EphA2 mRNA at a level that was 85% of the expression level of treated cells. Thus, EphA2 mRNA levels were increased with EGF treatment as compared to control cells not treated with EGF (FIG. 1A). The GAPDH PCR product is not shown.

Western blot analysis of whole cell lysates was performed with the EphA2-specific monoclonal antibody D7 to determine EphA2 protein expression levels in both untreated and EGF-treated cells. EphA2 protein levels were increased with EGF treatment as compared to control cells not treated with EGF (FIG. 1B).

6.2 Preparation of Monoclonal Antibodies

Immunization and Fusion

Monoclonal antibodies against the extracellular domain of EphA2 were generated using the fusion protein EphA2-Fc. This fusion protein consisted of the extracellular domain of human EphA2 linked to human immunoglobulin to facilitate secretion of the fusion protein.

Two groups of 5 mice each (either Balb/c mice (group A) or SJL mice (group B)) were injected with 10 μg of EphA2-Fc in TiterMax Adjuvant (total volume 100 μl) in the left metatarsal region at days 0 and 7. Mice were injected with 10 μg of EphA2-Fc in PBS (total volume 100 μl) in the left metatarsal region at days 12 and 14. On day 15, the popliteal and inguinal lymph nodes from the left leg and groin were removed and somatically fused (using PEG) with P3XBcl-2-13 cells.

Antibody Screening

Supernatants from bulk culture hybridomas were screened for immunoreactivity against EphA2 using standard molecular biological techniques (e.g., ELISA immunoassay). Supernatants can be further screened for the ability to inhibit an EphA2 monoclonal antibody (e.g., Eph099B-102.147, Eph099B-208.261, or Eph099B-210.248 deposited with the ATCC on Aug. 7, 2002 and assigned accession numbers PTA-4572, PTA-4573, and PTA-4574, respectively; B233; see also U.S. Provisional Patent Application No. 60/379,322 filed May 10, 2002, entitled “EphA2 Monoclonal Antibodies and Methods of Use Thereof” and U.S. patent application Ser. No. 10/436,783, filed May 12, 2003, entitled “EphA2 Agonistic Monoclonal Antibodies and Methods of Use Thereof”) from binding to EphA2.

6.3. EphA2 Monoclonal Antibodies Decrease EphA2 Function

6.3.1. EphA2 Phosphorylation and Degradation

EphA2 antibodies promoted tyrosine phosphorylation and degradation of EphA2 in MDA-MB-231 cells. Monolayers of cells were incubated in the presence of EphA2 antibodies or control for 8 minutes at 37° C. Cell lysates were then immunoprecipitated with an EphA2-specific antibody (D7, purchased from Upstate Biologicals, Inc., Lake Placid, N.Y. and deposited with the American Type Tissue Collection on Dec. 8, 2000, and assigned accession number PTA 2755), resolved by SDS-PAGE and subjected to western blot analysis with a phosphotyrosine-specific antibody (4G10, purchased from Upstate Biologicals, Inc., Lake Placid, N.Y.). The membranes were stripped and re-probed with the EphA2-specific antibody used in the immunoprecipitation (D7) as a loading control.

Western blot analyses and immunoprecipitations were performed as described previously (Zantek et al., 1999, Cell Growth Diff 10:629-38). Briefly, detergent extracts of cell monolayers were extracted in Tris-buffered saline containing 1% Triton X-100 (Sigma, St. Louis, Mo.). After measuring protein concentrations (BioRad, Hercules, Calif.), 1.5 mg of cell lysate was immunoprecipitated, resolved by SDS-PAGE and transferred to nitrocellulose (Protran, Schleicher and Schuell, Keene, N.H.). Antibody binding was detected by enhanced chemiluminescence (Pierce, Rockford, Ill.) and autoradiography (Kodak X-OMAT; Rochester, N.Y.). Levels of EphA2 phosphorylation were found to increase with incubation of some of the antibodies (data not shown).

Monolayers of MDA-MB-231 cells were incubated in the presence of presence of the antibodies of the invention or a control for either 4 hours or 24 hours at 37° C. Cell lysates were then resolved by SDS-PAGE and subjected to western blot analysis with an EphA2-specific antibody (D7). Many of the antibodies cause EphA2 protein levels to decrease (data not shown).

6.4. Kinetic Analysis of EphA2 Antibodies

The BIACORE assay was used to measure the K_{off} rates of the monoclonal antibodies of the invention. IgG present in the hybridoma supernatant was used for measurement.

Immobilization of EphA2

EphA2-Fc was immobilized to a surface on a CM5 sensorchip using a standard amine (70 μl of a 1:1 mix of NHS/EDC) coupling chemistry. Briefly, a 400 nM solution of EphA2-Fc in 10 mM NaOAc, pH4, was then injected over the activated surface to a density of 1000-1100 RU's. Unused reactive esters were subsequently “capped” with a 70 μl injection of 1M Et-NH2. Similarly, an activated and “capped” control surface was prepared on the same sensor chip without protein to serve as a reference surface.

Binding Experiments

A 250 μl injection of each of the EphA2 hybridoma supernatants was made over both the EphA2-Fc and control surfaces, and the binding responses were recorded. These supernatants were used undiluted. Following each injection, 10 min. of dissociation phase data was collected. Purified EphA2 monoclonal antibody EA2 (a hybridoma producing EA2 was deposited with the American Type Culture Collection on May 22, 2002 and assigned accession number PTA-4380) was prepared to serve as a positive control (at 1 μg, 5 μg and 25 μg per 250 μl of growth medium). A negative control monoclonal antibody was also prepared at 5 μg/250 μl growth medium. Control injections of growth medium across these surfaces were also made. Following each binding cycle, the EphA2-Fc surface was regenerated with a single 1 min. pulse (injection) of 1M NaCl-50 mM NaOH.

Data Evaluation

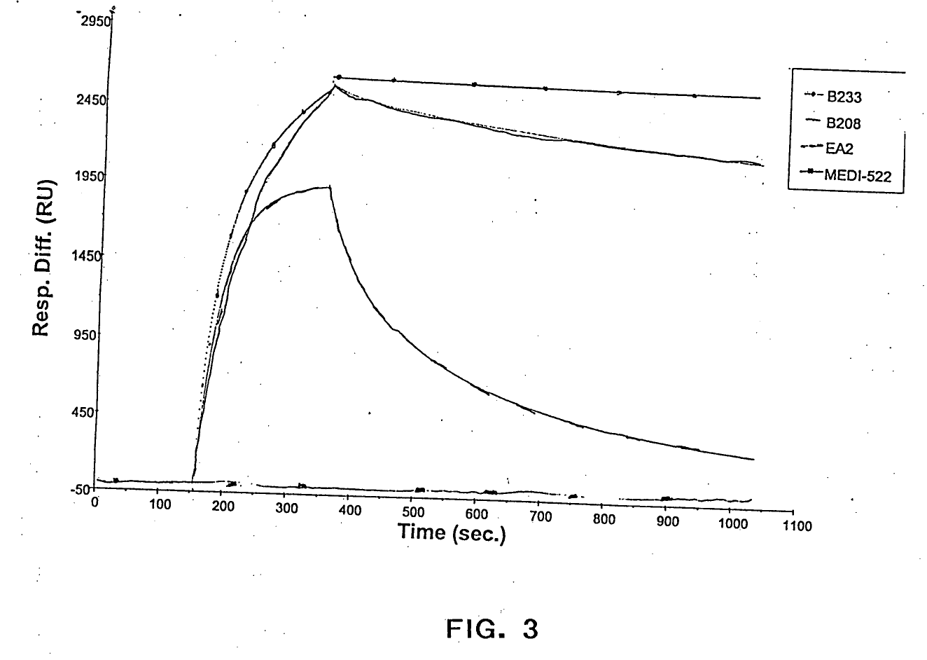

The binding data was corrected by subtracting out both artifactual noise (blank medium injections) and non-specific binding (control surface), in a technique known as “double-referencing.” Thus the sensorgram overlays represent “net” binding curves. Eph099B-208.261 and B233 have slower K_{off} rates than EA2 (FIG. 3). Additionally, other antibodies of the invention have slow K_{off} rates including Eph099B-102.147 and Eph099B-210.248 (data not shown).

6.5. EphA2 Expression on Lung Epithelium In Vivo

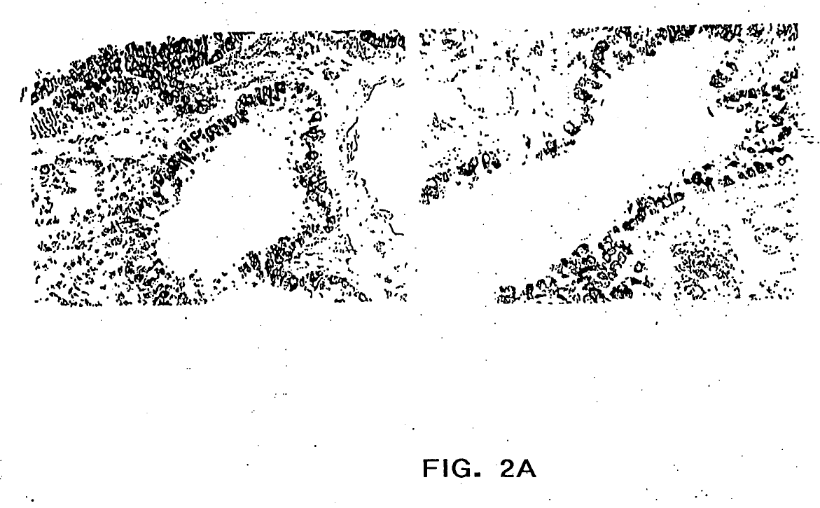

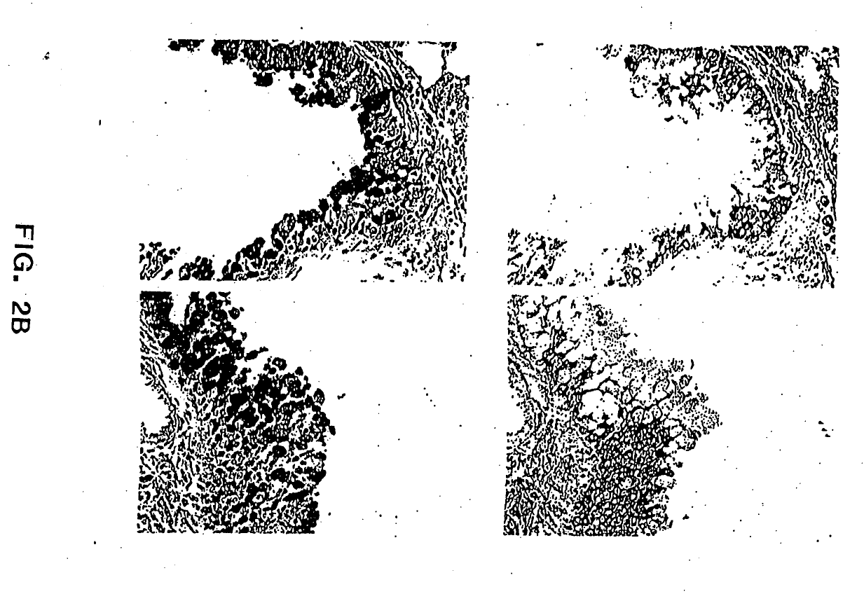

Normal BALB/c mice were euthanized by CO₂ asphyxiation. Lung tissue was preserved by carefully inflating the tissue with 10% buffered formalin before embedding in paraffin blocks and sectioning. Deparaffinized 10 micron sections were incubated with a 1:100 dilution of a polyclonal rabbit serum directed against murine EphA2. Bound antibody was detected with biotin-conjugated anti-rabbit antibodies (1:500 dilution) followed by streptavidin-horseradish peroxidase conjugate (1:1000). Bound horseradish peroxidase was visualized with diaminobenzidine (DAB) staining. Epithelial cells of only the basal layer showed expression of EphA2 (FIG. 2A).

EphA2 expression was also determined in RSV-infected mice. On day 0, normal BALB/c mice were intraperitoneally immunized with 15 μg of formalin-inactivated respiratory syncytial virus (FI-RSV) adsorbed onto Alum adjuvant. An identical dose of FI-RSV was administered on day 5. On day 12, the mice were intranasally challenged with live RSV, at a concentration of 10⁶ plaque forming units (pfu) in 100 ml volume. Mice were euthanized and lung tissue processed as described previously. In addition to EphA2 staining, tissue was stained with periodic acid-Schiff (PAS) reagent according to standard techniques to visualize goblet cells. As in uninfected lung tissue, epithelial cells of only the basal layer showed expression of EphA2 (FIG. 2B, right panel). Mucin-secreting goblet cells do not express EphA2 (FIG. 2B, left panel).

6.6. Decreased EphA2 Levels Using EphA2 Antisense Oligonucleotides

An antisense oligonucleotide-based approach that decreased EphA2 expression in epithelial cells independent of EphA2 activation was developed. To decrease EphA2 protein levels, MDA-MB-231 breast carcinoma cells were transiently transfected with phosphorothioate-modified antisense oligonucleotides that corresponded to a sequence that was found to be unique to EphA2 as determined using a sequence evaluation of GenBank (5′-CCAGCAGTACCGCTTCCTTGCCCTGCGGCCG-3′; SEQ ID NO:44). Inverted antisense oligonucleotides (5′-GCCGCGTCCCGTTCCTTCACCATGACGACC-3′; SEQ ID NO:45) provided a control. The cells were transfected with oligonucleotides (2 μg/ml) using Lipofectamine PLUS Reagent (Life Technologies, Inc.) according to the manufacturer's protocol. Twenty-four hours post-transfection, the cells were extracted and subjected to western blot analysis.

Western blot analyses and immunoprecipitations were performed as described previously (Zantek et al., 1999, Cell Growth Diff. 10:629-38). Briefly, detergent extracts of cell monolayers were extracted in Tri s-buffered saline containing 1% Triton X-100 (Sigma, St. Louis, Mo.). After measuring protein concentrations (BioRad, Hercules, Calif.), 1.5 mg of cell lysate was immunoprecipitated, resolved by SDS-PAGE and transferred to nitrocellulose (Protran, Schleicher and Schuell, Keene, N.H.). EphA2 was detected with an EphA2-specific antibody (D7, purchased from Upstate Biologicals, Inc., Lake Placid, N.Y.). To control for sample loading, the membranes were stripped and re-probed with paxillin antibodies (a gift from Dr. K. Burridge at the University of North Carolina). Antibody binding was detected by enhanced chemiluminescence (Pierce, Rockford, Ill.) and autoradiography (Kodak X-OMAT; Rochester, N.Y.).

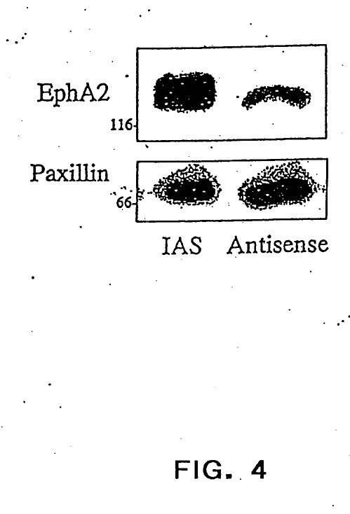

Western blot analyses confirmed that antisense oligonucleotides selectively decreased EphA2 expression in MDA-MB-231 cells whereas an inverted antisense control (IAS) did not (FIG. 4).

6.7. Treatment of Patients with Asthma or COPD

A study is designed to assess pharmacokinetics and safety of monoclonal antibodies of the invention in patients with asthma or COPD. Patients are administered a single dose of a monoclonal antibody of the invention via either intravenous or pulmonary administration and then, beginning 4 weeks later, are analyzed following administration of repeated weekly doses at the same dose via the same administration route over a period of 12 weeks. The safety of treatment with the monoclonal antibody of the invention is assessed as well as potential changes in disorder activity over 26 weeks of dosing. Different groups of patients are treated and evaluated similarly but receive doses of 1 mg/kg, 2 mg/kg, 4 mg/kg, or 8 mg/kg.

Changes are measured or determined by the incident and severity of respiratory symptoms.

6.8. Role of EphA2 in Progression of Fibrosis

For an in vitro model of fibrosis, Beas-2B cells (bronchial epithelium cells transformed with SV40 virus) were treated with bleomycin (25-100 mUnits/ml). After 5 hrs, increases in IL-6 and IL-8 were detected. This response is typical of damaged epithelium. After 24 hrs, increases in Fas, a receptor that mediates apoptosis, were detected. Increases in apoptosis (via increases in annexin V binding) and cell death, in general (as detected via propidium iodide uptake), were also detected. Immunostaining using an anti-phosphotyrosine antibody showed changes in cellular morphology and adhesion properties after 24 hr of bleomycin treatment. EphA2 upregulation at 24 hrs post-treatment (via western blot and FACS analysis) was also detected. Although bleomycin treatment caused increases in EphA2 levels, phosphorylation of tyrosine kinase was greatly decreased in these cells, suggesting altered function of the molecule.

6.8.1 Materials and Methods

For in vitro testing, Beas-2B bronchial epithelium cells (ATCC Catalog No. CRL-9609) were used. To create the cell line, epithelial cells were isolated from normal human bronchial epithelium obtained from autopsy of non-cancerous individuals. The cells were infected with an adenovirus 12-SV40 virus hybrid (Adl2SV40) and cloned. The cells retain the ability to undergo squamous differentiation in response to serum, and can be used to screen chemical and biological agents for ability to induce or affect differentiation and/or carcinogenesis. The cells stain positively for keratins and SV40 T antigen (Reddel, et al., Immortalized human bronchial epitherial mesothelial cell lines. U.S. Pat. No. 4,885,238, issued Dec. 5, 1989).

Immunofluorescence. Cells were grown on glass coverslips to visualize individual cells. At a density of ˜70% confluence, cells were treated with 25 mUnits/ml bleomycin or vector (PBS). After 24 hours, samples were fixed in 3.7% formaldehyde solution, extracted in 0.5% Triton X-100, and stained using the anti-phosphotyrosine clone, PY20 (Upstate; Charlottesville, Va.). Immunostaining was visualized using phycoerythrin-conjugated donkey antimouse antibodies (BD Biosciences; San Jose, Calif.) and epifluorescence microscopy.

Western Blot Analysis. Cell monolayers were extracted in a buffer containing 1% Triton-X-100 for 5 minutes on ice. After protein concentrations were measured by Coomassie Blue staining (Pierce; Rockford, Ill.), equal amounts of protein were resolved by SDS-PAGE and transferred to nitrocellulose (Protran; Schleicher & Schuell; Keene, N.H.). Antibody binding was detected by enhanced chemiluminescence as recommended by the manufacturer (Pierce).

Immunoprecipitation. Immunoprecipitation experiments were performed for 2.5 hours at 4° C. using the EphA2 antibody, D7 (Upstate; Charlottesville, Va.) and rabbit antimouse (Chemicon) conjugated protein A-Sepharose (Sigma). Immunoprecipitations were washed three times in lysis buffer, resuspended in SDS sample buffer (Tris buffer containing 5% SDS, 3.8% DTT, 25% glycerol, and 0.1% bromophenol blue), and resolved by 10% SDS-PAGE.

Luminex Analysis of Cytokines Produced by BEAS-2B Cells after Exposure to Bleomycin Sulfate. Materials used: Bleomycin sulfate, Sigma Cat. # B2434, Lot 102K0753, 1.8 U/mg, 20 mg; Beadlyte Human Multicytokine Beadmaster Kit, Upstate Cat. # 48-100, Lot 26301; Human IL-6 Beadmates, Upstate Cat. # 46-106, Lot 24204; Human IL-8 Beadmates, Upstate Cat. # 46-108, Lot 24205; Luminex 100 instrument; BEAS-2B cells, ATCC Cat. # CRL-9609; BEGM Bullet kit (growth medium), Cambrex Cat. # CC3170.

BEAS-2B cells were plated in a 96-well plate at 3×10⁴/well in BEGM/10% FBS. The next day, medium was removed in duplicate and replaced with the same medium containing dilutions of bleomycin (100, 50, 25, 10, and 0 mU/ml). After 5 hours incubation at 37° C., 5% CO₂, the supernatants were collected, centrifuged 500×g for 3 minutes at room temperature, and stored at −20° C. Cytokine production in the cell supernatants was analyzed according to the Beadmaster kit directions using the Luminex 100.

Apoptosis assays. 2^{e}5 cells per well Beas-2B cells were plated on 6 well tissue-culture-treated plates. Cells were allowed to attach to wells overnight. The next day, 100 mU/mL bleomycin was added to wells. After 24 hour bleomycin exposure, cells were detached with 0.25% trypsin, centrifuged at 300×g and washed with normal cell culture medium. Annexin V binding assay was performed using the Annexin-V FITC Apoptosis Detection Kit (BD Biosciences Pharmingen, San Diego, Calif.). Annexin V binding and PI incorporation was measured using FACSCalibur Flow Cytometer (BD Biosciences, San Jose, Calif.)

6.8.2 Results

MCF-10A is a non-transformed epithelial system, which can allow for analysis of cellular adhesions using immunostaining of the cytoskeleton (Kinch et al., 1995, J. Cell. Biol. 130(2):461-71). As such, these cells were used to show that overexpression of EphA2 increased cell-ECM attachments. Upregulation of EphA2 can result in morphological changes, similar to those seen in bleomycin-treated epithelium (in which EphA2 is also upregulated. Similarly, EphA2 overexpression increases fibronectin expression and thereby increases cell-ECM attachments. Epithelium produces fibronectin during the initial wound healing response so this suggests that EphA2 upregulation is upstream of this event in wound healing-fibrosis. In the inverse experiment with MDA-MB-231, treatment of a cell that has high endogenous levels of fibronectin (e.g., MDA-MB-231) with EphA2 antibodies is sufficient to decrease fibronectin levels.

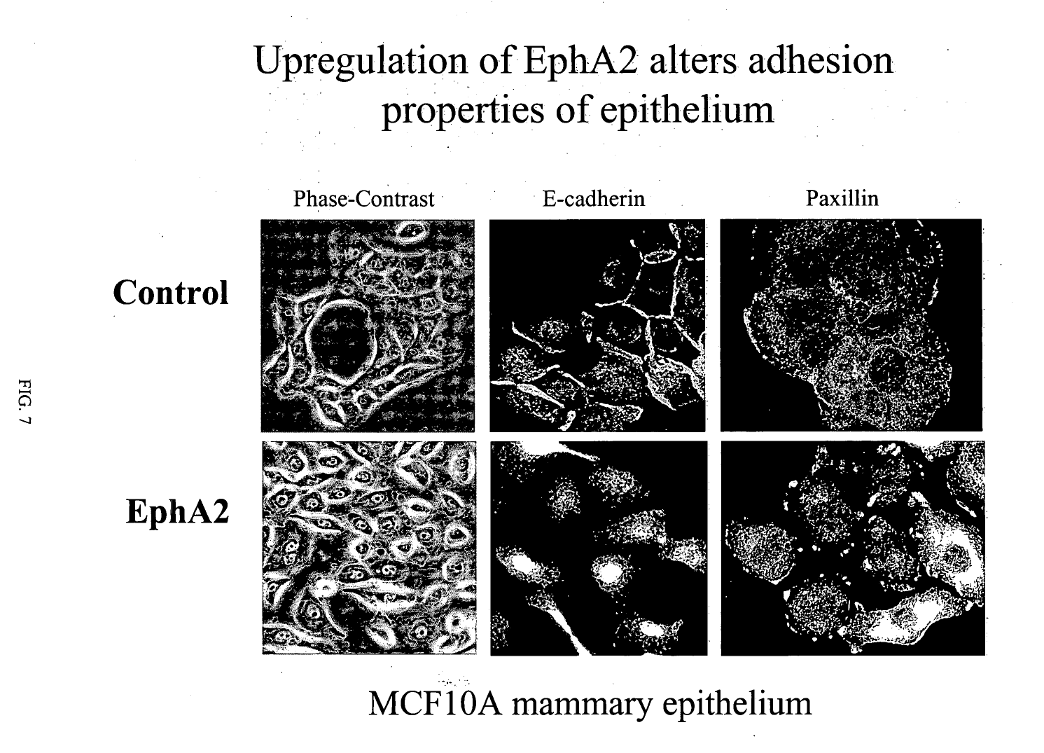

MCF10A mammary epithelial cells were examined by phase-contrast microscopy and fluorescence microscopy with E-cadherin and Paxillin staining. Microscopic analysis revealed decreased cell-cell adhesion in EphA2-upregulated cells relative to control cells (FIG. 7), indicating that upregulation of EphA2 alters the adhesion properties of the epithelium.

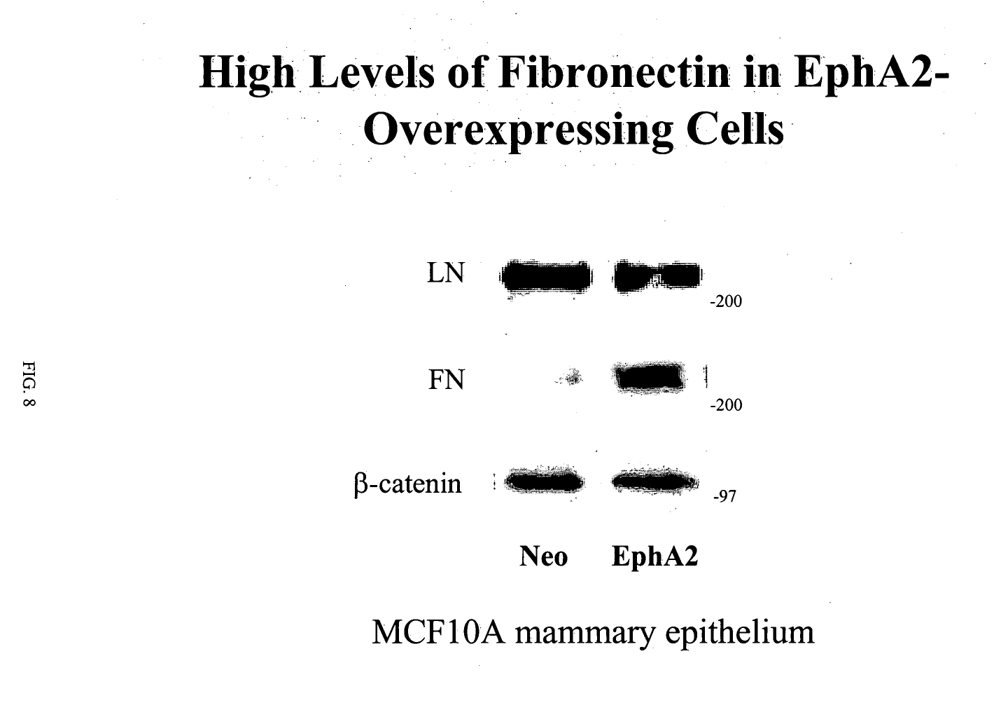

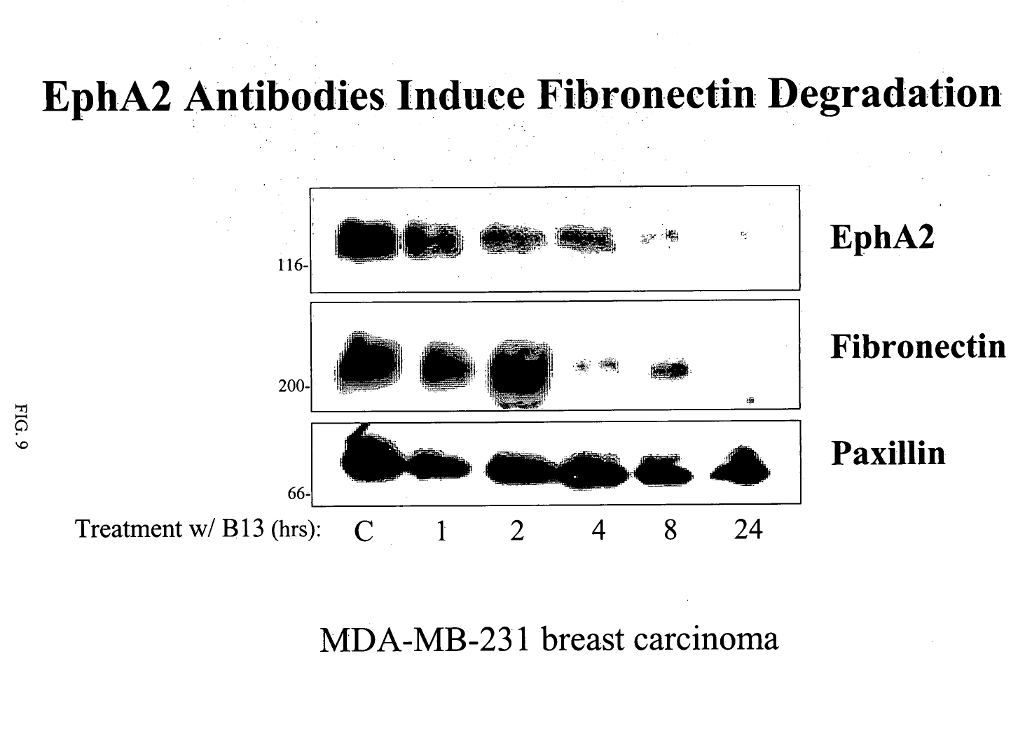

Western Blot of extracts from MCF10A mammary epithelial cells (FIG. 8) overexpressing Neo (lane 1) or EphA2 (lane 2) showed elevated fibronectin expression with increased EphA2 expression, indicating that EphA2-overexpressing cells have increased levels of fibronectin. A Western Blot of extracts from MDA-MB-231 breast carcinoma cells treated with B13 EphA2 antibodies (FIG. 9) showed decreased EphA2 protein levels and degradation of fibronectin over a 24 hour period relative to paxillin protein levels which remain stable over time, indicating that EphA2 antibodies induce fibronectin degradation.

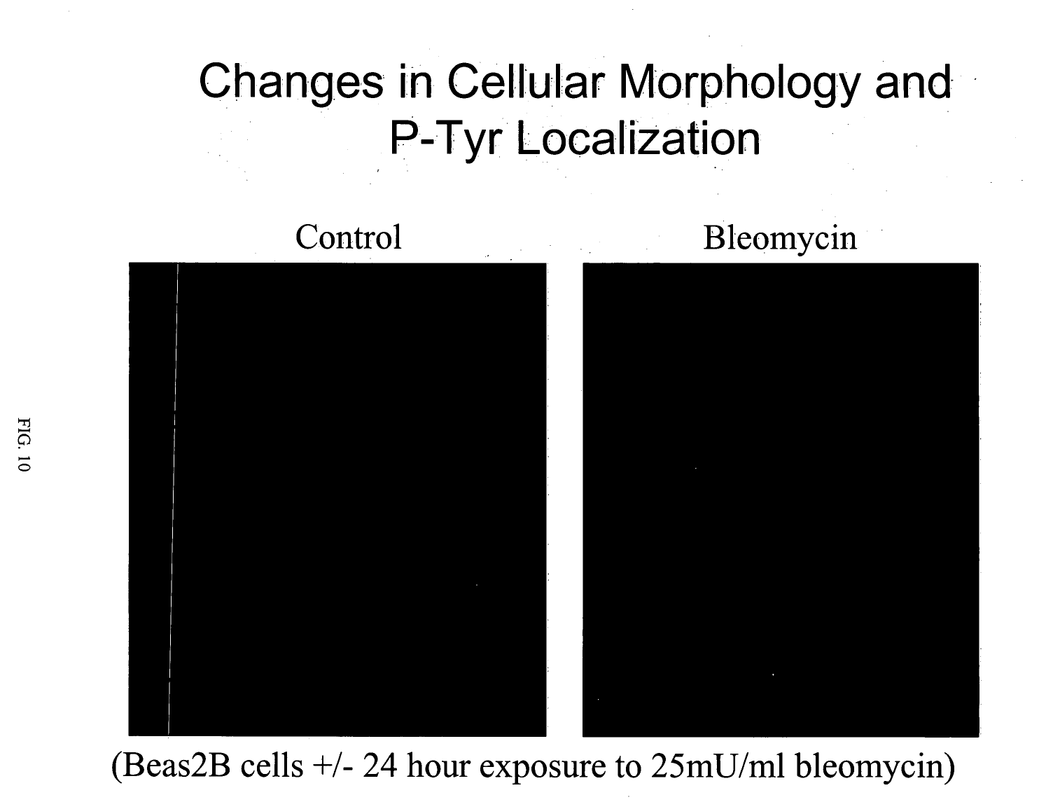

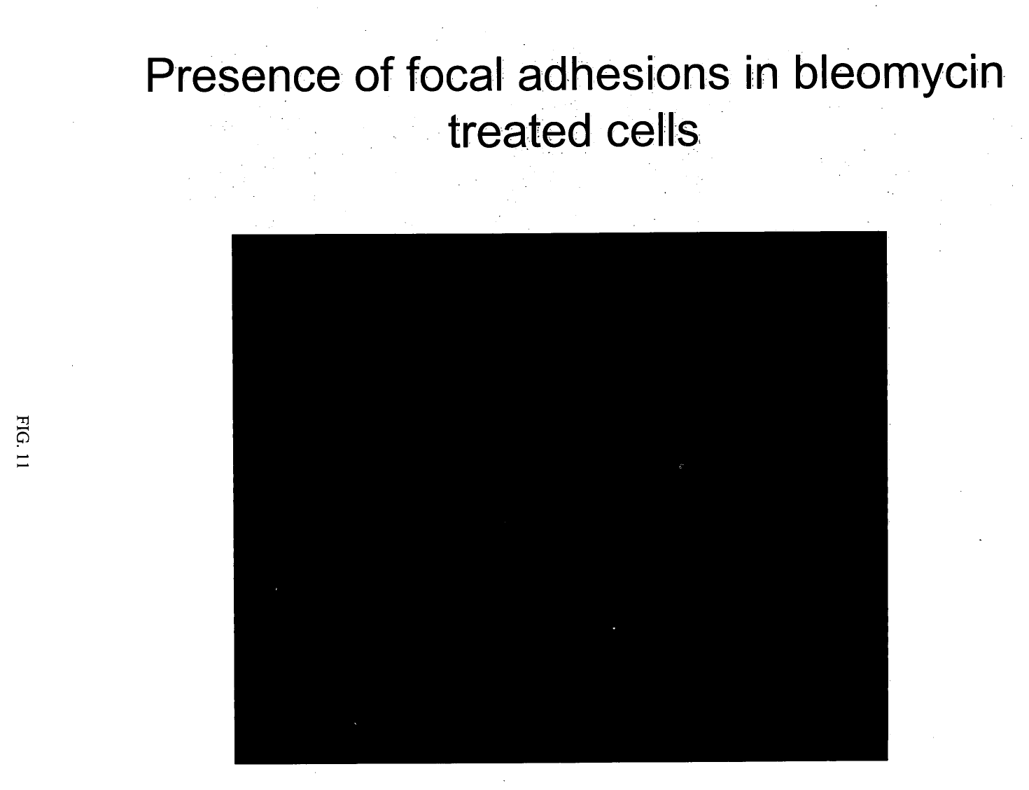

Fluorescence microscopy of Beas2B cells (FIG. 10) stained to reveal phosphorylated tyrosine (P-Tyr) showed that P-Tyr is highly localized to sites of cellular adhesion (e.g., focal adhesions) in cells treated for 24 hours with bleomycin relative to untreated control cells, indicating changes in cellular morphology and P-Tyr localization resulting from bleomycin treatment. Bleomycin-treated Beas2B cells further showed more prominent focal adhesions (FIG. 11) than matched control cells that had not been treated with bleomycin.

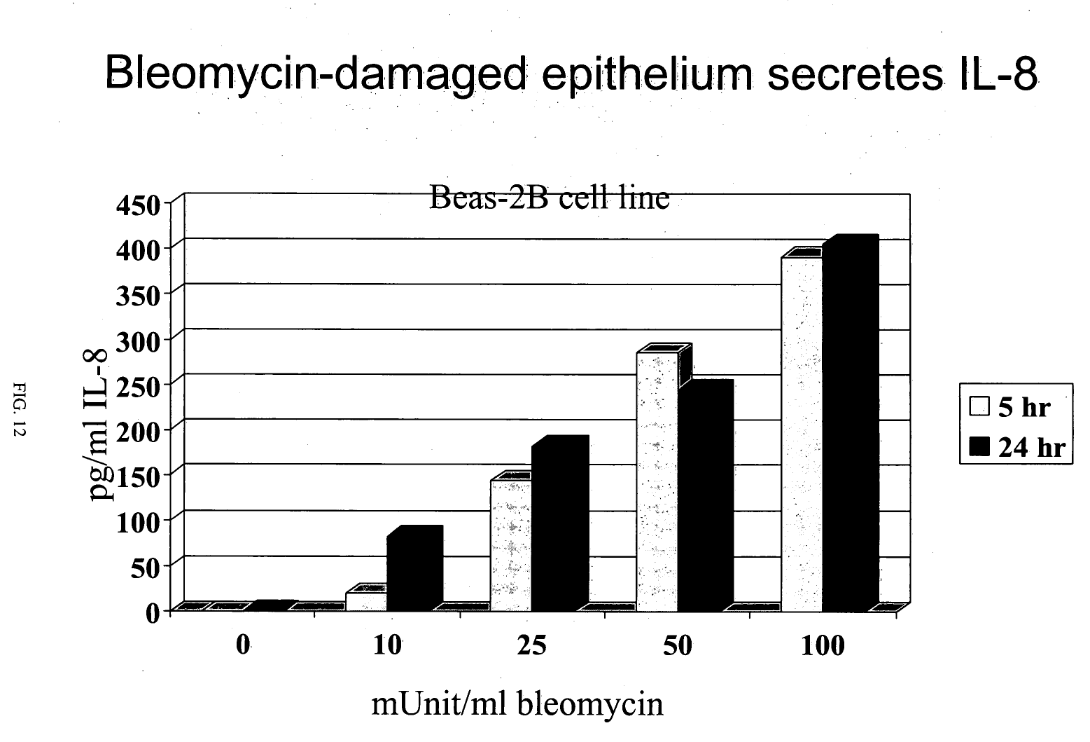

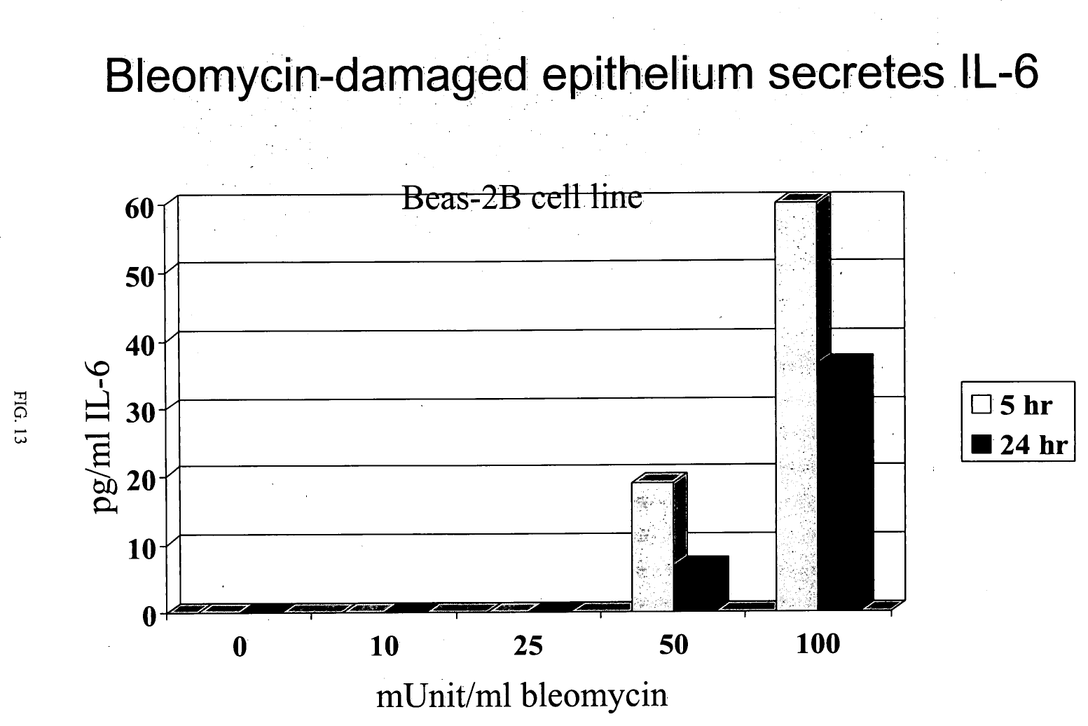

Beas-2B cells treated with increasing amounts of bleomycin secreted increasing levels of IL-8 (FIG. 12) and IL-6 (FIG. 13) over a 24-hour period, indicating that bleomycin-damaged epithelium has an enhanced immunosecretory response. Secretion of other cytokines and factors such as IL-1α, IL-β, IL-7, TNF-α, Eotaxin, MCP-1, Rantes, and MIP-1 were also tested; no changes in the levels of these were detected.

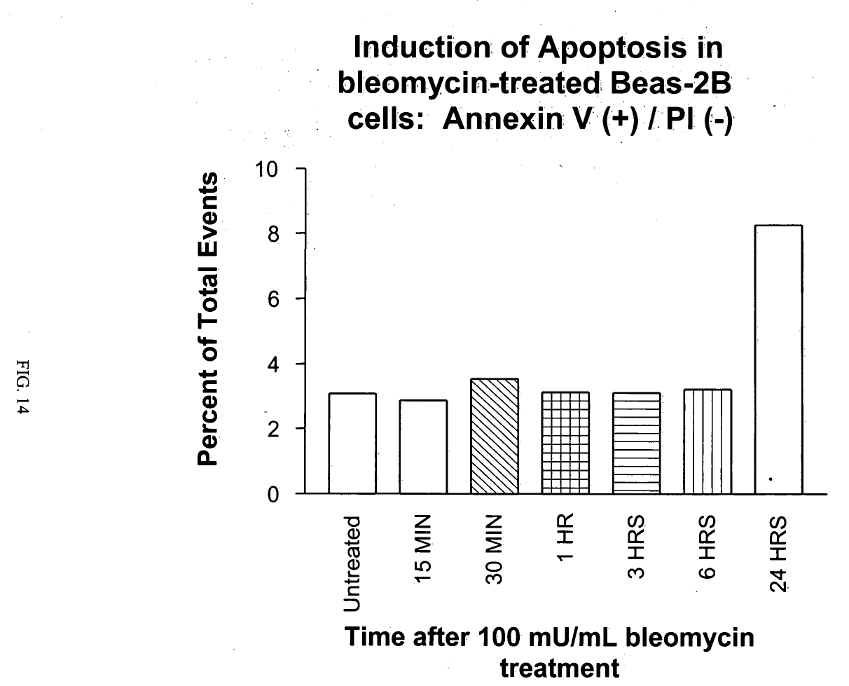

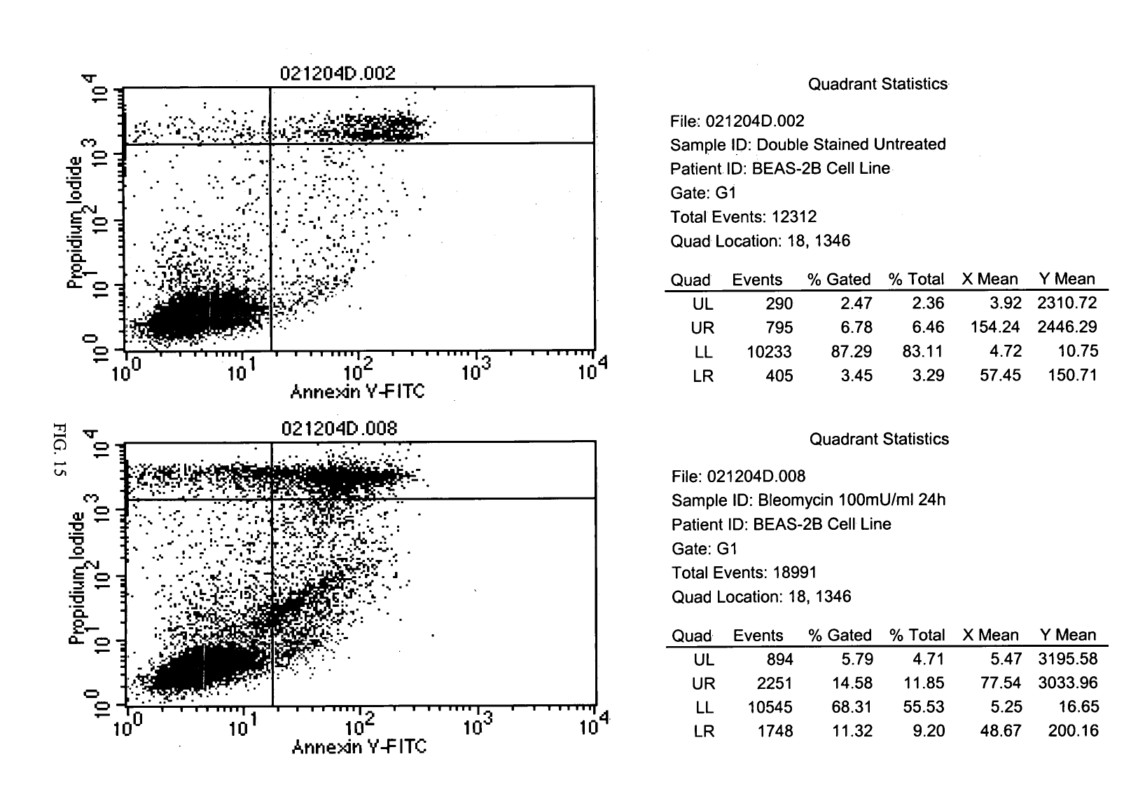

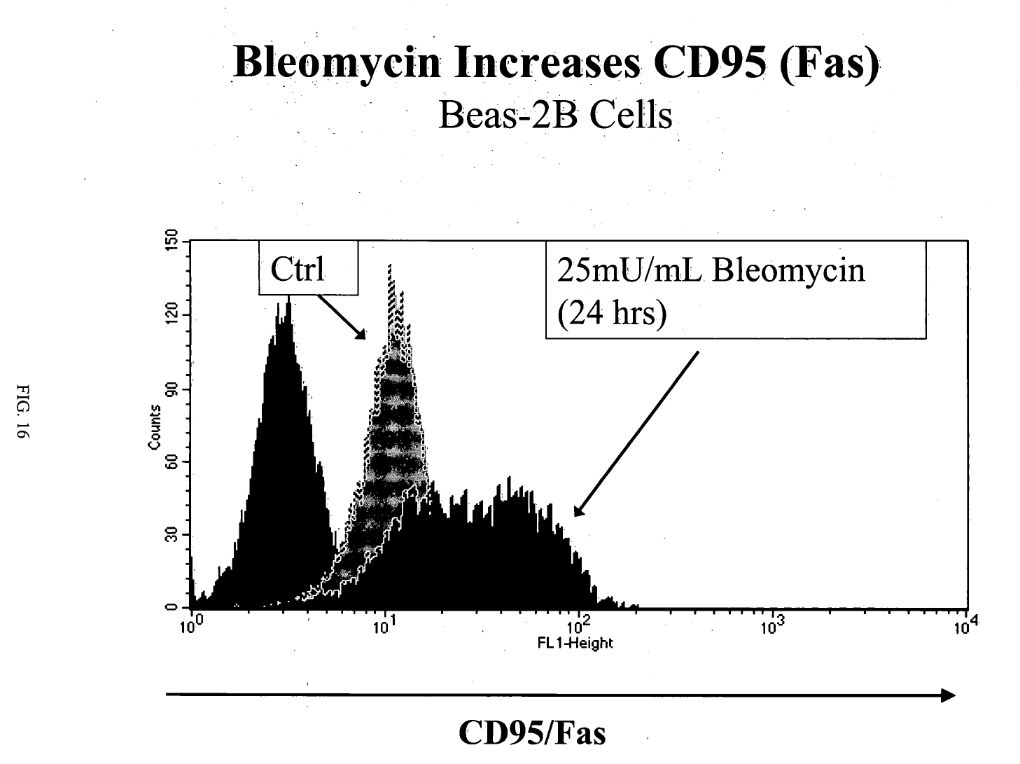

Analysis of Beas-2B cells by Fluorescence-Activated Cell Sorter (FACS) (FIG. 14) showed increased apoptotic events as determined by annexin V binding assays 24 hours after bleomycin treatment relative to untreated control cells, indicating induction of apoptosis in these cells. FACS analysis of Beas-2B cells showed increased CD95/Fas expression 24 hours after treatment with bleomycin (FIG. 16) relative to untreated control cells, indicating that bleomycin increases CD95 (Fas) expression.

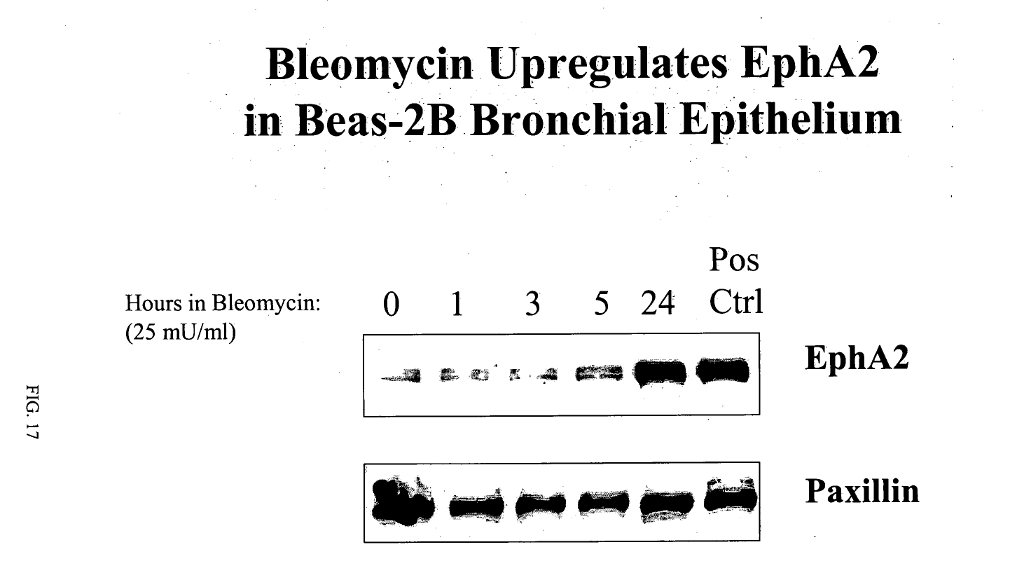

Western Blot analysis of Beas-2B bronchial epithelial cells showed increased EphA2 expression after 24 hours of treatment with bleomycin (FIG. 17), compared to expression levels of paxillin, a cytoskeletal protein that is expressed at equivalent levels in control and treated samples and thus is used to control for equal sample loading. Paxillin levels remained stable, indicating that bleomycin specifically upregulates EphA2 in Beas-2B bronchial epithelium.

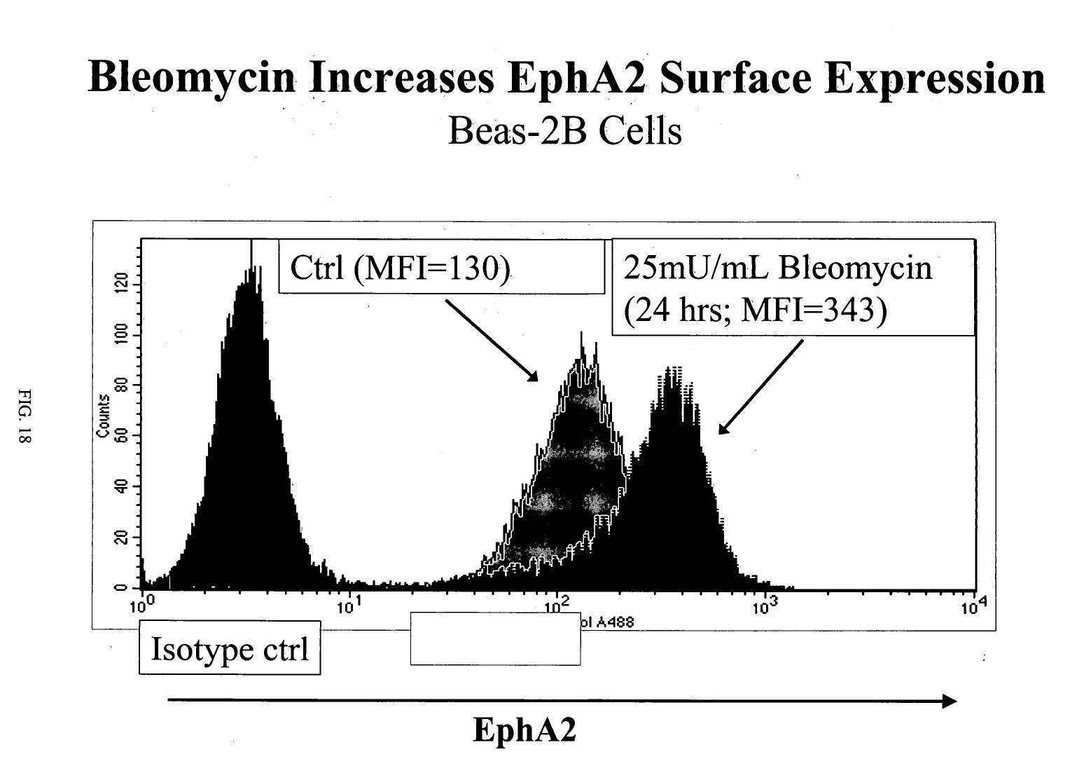

FACS analysis of Beas-2B cells showed increased EphA2 surface expression 24 hours after treatment with bleomycin relative to untreated control cells (FIG. 18), indicating that bleomycin increases EphA2 expression in bronchial epithelium cells.

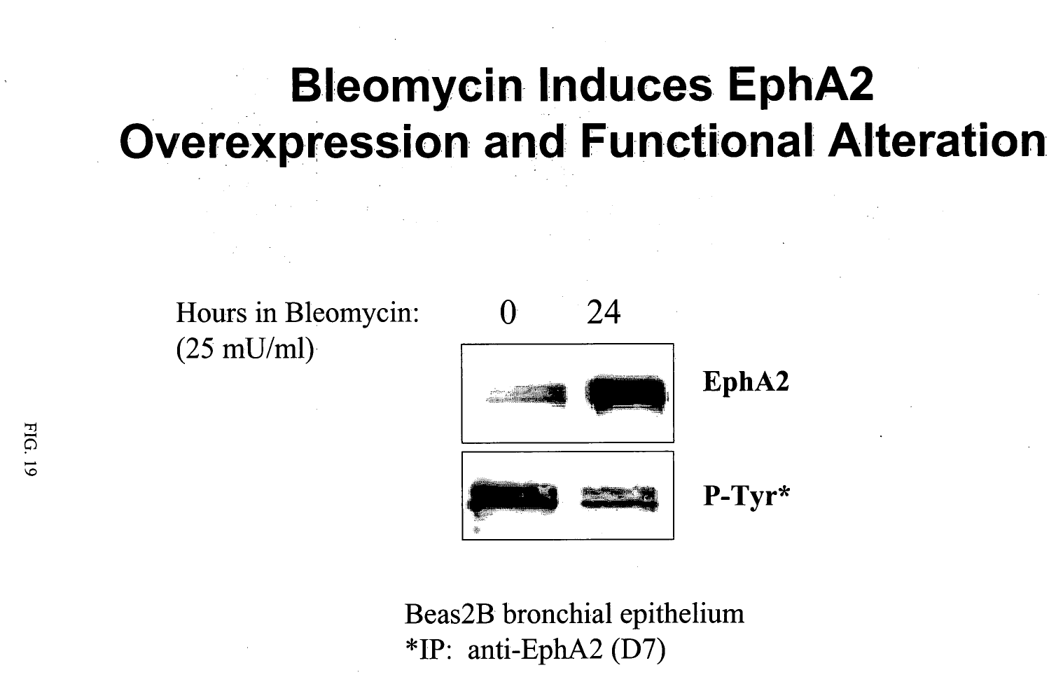

Western Blot analysis of Beas-2B bronchial epithelial cells showed increased EphA2 expression after 24 hours of treatment with bleomycin (FIG. 19), indicating upregulation of EphA2, while P-Tyr levels decrease slightly, indicating altered function of EphA2.

7. Equivalents

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

1. A method of treating a non-neoplastic hyperproliferative cell or excessive cell accumulation disorder in a patient in need thereof, said method comprising administering to said patient a therapeutically effective amount of an EphA2 agonistic agent, wherein said EphA2 agonistic agent binds EphA2 and increases EphA2 cytoplasmic tail phosphorylation, increases EphA2 autophosphorylation, increases EphA2 degradation, reduces a pathology-causing cell phenotype, or reduces EphA2 activity wherein said activity is not autophosphorylation.

2. The method of claim 1 wherein said non-neoplastic hyperproliferative cell or excessive cell accumulation disorder is a hyperproliferative epithelial cell disorder selected from the group consisting of asthma, chronic pulmonary obstructive disease, lung fibrosis, asbestosis, IPF, DIP, UIP, kidney fibrosis, liver fibrosis, other fibroses, bronchial hyper responsiveness, psoriasis, and seborrheic dermatitis.

3. The method of claim 2, wherein a pathology-causing cell phenotype of said hyperproliferative epithelial cell disorder is secretion of mucin, differentiation of an EphA2-expressing cell into a mucin-secreting cell, secretion of inflammatory factors, or epithelial or endothelial cell hyperproliferation.

4. The method of claim 1 wherein said non-neoplastic hyperproliferative cell or excessive cell accumulation disorder is a hyperproliferative endothelial cell disorder selected from the group consisting of restenosis, hyperproliferative vascular disease, Behcet's Syndrome, atherosclerosis, and macular degeneration.

5. The method of claim 1 wherein said non-neoplastic hyperproliferative cell or excessive cell accumulation disorder is a hyperproliferative fibroblast cell disorder.

6. The method of claims 4 or 5, wherein a pathology-causing cell phenotype of said hyperproliferative endothelial cell disorder is increased cell migration, cell volume, secretion of extracellular matrix molecules, secretion of matrix metalloproteinases, or endothelial cell hyperproliferation.

7. The method of claim 1 wherein said EphA2 agent is an antibody or antigen binding fragment thereof.

8. The method of claim 1 wherein said EphA2 agent is chosen from the group consisting of small molecule agonists, enzymatic activity antagonists, ribozymes, siRNA, and EphA2 antisense molecules.

9. The method of claim 7 wherein the said antibody is a monoclonal antibody.

10. The method of claim 9 wherein said monoclonal antibody binds EphA2 with a K_{off} of less than 3×10⁻³ s⁻¹ under conditions appropriate for antibody-EphA2 binding.

11. The method of claim 9 wherein said monoclonal antibody is Eph099B-102.147, Eph099B-208.261, Eph099B-210.248, or B233 or comprises a CDR from Eph099B-102.147, Eph099B-208.261, Eph099B-210.248, or B233.

12. The method of any of claims 7, 9, or 10 wherein said monoclonal antibody is a human antibody.

13. The method of any of claims 7, 9, 10, or 11 wherein said monoclonal antibody is humanized.

14. The method of claim 1 wherein said administration increases EphA2 phosphorylation in a treated cell relative to the level of EphA2 phosphorylation in an untreated cell.

15. The method of claim 1 wherein said administration decreases EphA2 expression in a treated cell relative to the level of EphA2 expression in an untreated cell.

16. The method of claim 1 further comprising the administration of one or more additional non-neoplastic hyperproliferative cell or excessive cell accumulation disorder therapies.

17. The method of claim 16, wherein said pathology-causing epithelial or endothelial cell phenotype is secretion of mucin, differentiation of an EphA2-expressing cell into a mucin-secreting cell, secretion of fibronectin, secretion of inflammatory factors, or epithelial or endothelial cell hyperproliferation.

18. A method of treating asthma or chronic obstructive pulmonary disease in a patient in need thereof, said method comprising administering to said patient a therapeutically effective amount of one or more EphA2 agonistic agents, wherein said EphA2 agonistic agent binds EphA2 and increases EphA2 cytoplasmic tail phosphorylation, increases EphA2 autophosphorylation, increases EphA2 degradation, reduces a pathology-causing cell phenotype, or reduces EphA2 activity wherein said activity is not autophosphorylation.

19. A method of treating restenosis in a patient in need thereof, said method comprising administering to said patient a therapeutically effective amount of one or more EphA2 agents, wherein said EphA2 agent binds EphA2 and increases EphA2 cytoplasmic tail phosphorylation, increases EphA2 autophosphorylation, increases EphA2 degradation, reduces a pathology-causing cell phenotype, or reduces EphA2 activity wherein said activity is not autophosphorylation.

20. The method of claim 19, wherein said pathology-causing endothelial cell phenotype is cell migration, cell volume, secretion of extracellular matrix molecules, secretion of matrix metalloproteinases, or endothelial cell hyperproliferation.

21. The method of claim 18 or 19 wherein said EphA2 agent is an antibody or antigen binding fragment thereof.

22. The method of claim 21 wherein the said antibody is a monoclonal antibody.

23. The method of claim 22 wherein said monoclonal antibody binds EphA2 with a K_{off} of less than 3×10⁻³ s⁻¹ under conditions appropriate for antibody-EphA2 binding.

24. The method of claim 22 wherein said monoclonal antibody is Eph099B-102.147, Eph099B-208.261, Eph099B-210.248, or B233.

25. The method of any of claims 21, 22, or 23 wherein said monoclonal antibody is a human antibody.

26. The method of any of claims 21, 22, 23, or 24 wherein said monoclonal antibody is humanized.

27. The method of any of claims 1, 15, or 17 further comprising the administration of one or more immunomodulatory agents.

28. The method of claim 27 wherein said immunomodulatory agent is an antibody that immunospecifically binds IL-9.

29. The method of any of claims 1 or 17 further comprising the administration of one or more anti-viral agents.

30. The method of claim 29 wherein said anti-viral agent is an anti-RSV agent.

31. A method of diagnosing a non-neoplastic hyperproliferative cell or excessive cell accumulation disorder or monitoring the efficacy of therapy for a non-neoplastic hyperproliferative cell or excessive cell accumulation disorder in a patient known to or suspected to have said disorder, said method comprising:
a) contacting cells of said patient with an EphA2 antibody that agonizes EphA2, decreases EphA2 activity, or decreases a pathology-causing cell phenotype; and
b) detecting EphA2 antibody binding to said cells,
wherein detecting a higher EphA2 antibody binding level than in a control patient that does not have a non-neoplastic hyperproliferative cell or excessive cell accumulation disorder indicates that the patient has a hyperproliferative cell or excessive cell accumulation disorder.

32. The method of claim 31 wherein said non-neoplastic hyperproliferative cell or excessive cell accumulation disorder is selected from the group consisting of asthma, chronic pulmonary obstructive disease, lung fibrosis, bronchial hyper responsiveness, psoriasis, seborrheic dermatitis, cystic fibrosis, restenosis, hyperproliferative vascular disease, Behcet's Syndrome, atherosclerosis, and macular degeneration.
